SEQUENCE LISTING (107 sequences)

SEQ ID NO: 1 - DNA, 25 bp, synthetic construct
  source 1..25: mol_type = other DNA
tgcctcctactgagcaatacttact

SEQ ID NO: 2 - DNA, 25 bp, synthetic construct
  source 1..25: mol_type = other DNA
aaaagccccacttttcatctgcttt

SEQ ID NO: 3 - DNA, 25 bp, synthetic construct
  source 1..25: mol_type = other DNA
cataggttgtagcacaccacagtct

SEQ ID NO: 4 - DNA, 25 bp, synthetic construct
  source 1..25: mol_type = other DNA
cttccctcccatctgatgtatattc

SEQ ID NO: 5 - DNA, 25 bp, synthetic construct
  source 1..25: mol_type = other DNA
actccttatctaggaaaagaaaagg

SEQ ID NO: 6 - DNA, 25 bp, synthetic construct
  source 1..25: mol_type = other DNA
ggcagggctccccatgtgctgtgcc

SEQ ID NO: 7 - DNA, 25 bp, synthetic construct
  source 1..25: mol_type = other DNA
ggcggtcaaagctggaatgacaaga

SEQ ID NO: 8 - DNA, 23 bp, synthetic construct
  source 1..23: mol_type = other DNA
  modified_base 1: mod_base = tm
  modified_base 2: mod_base = gm
  modified_base 3: mod_base = gm
  modified_base 4: mod_base = OTHER; note = 2' O-methyl adenosine
  modified_base 5: mod_base = OTHER; note = 2' O-methyl adenosine
  modified_base 19: mod_base = cm
  modified_base 20: mod_base = cm
  modified_base 21: mod_base = tm
  modified_base 23: mod_base = gm
  modified_base 22: mod_base = gm
tggaatctaaggattcatcctgg

SEQ ID NO: 9 - DNA, 23 bp, synthetic construct
  source 1..23: mol_type = other DNA
  modified_base 1: mod_base = OTHER; note = 2'-o-methyladenosine
  modified_base 2: mod_base = cm
  modified_base 3: mod_base = tm
  modified_base 4: mod_base = cm
  modified_base 5: mod_base = tm
  modified_base 23: mod_base = cm
  modified_base 22: mod_base = tm
  modified_base 21: mod_base = gm
  modified_base 20: mod_base = gm
  modified_base 19: mod_base = tm
actctgcctgcagtagtatggtc

SEQ ID NO: 10 - DNA, 22 bp, synthetic construct
  source 1..22: mol_type = other DNA
  modified_base 1: mod_base = OTHER; note = 2' O-methyl adenosine
  modified_base 2: mod_base = tm
  modified_base 3: mod_base = OTHER; note = 2'-o-methyladenosine
  modified_base 4: mod_base = tm
  modified_base 5: mod_base = cm
  modified_base 22: mod_base = cm
  modified_base 21: mod_base = cm
  modified_base 20: mod_base = cm
  modified_base 19: mod_base = tm
  modified_base 18: mod_base = tm
atatcttctgtgacactttccc

SEQ ID NO: 11 - DNA, 23 bp, synthetic construct
  source 1..23: mol_type = other DNA
  modified_base 1: mod_base = tm
  modified_base 2: mod_base = OTHER; note = 2'-o-methyladenosine
  modified_base 3: mod_base = cm
  modified_base 4: mod_base = tm
  modified_base 5: mod_base = tm
  modified_base 23: mod_base = tm
  modified_base 22: mod_base = gm
  modified_base 21: mod_base = OTHER; note = 2'-o-methyladenosine
  modified_base 20: mod_base = tm
  modified_base 19: mod_base = OTHER; note = 2'-o-methyladenosine
tactttctttggctcttcatagt

SEQ ID NO: 12 - DNA, 23 bp, synthetic construct
  source 1..23: mol_type = other DNA
  modified_base 1: mod_base = gm
  modified_base 2: mod_base = cm
  modified_base 3: mod_base = OTHER; note = 2'-o-methyladenosine
  modified_base 4..5: mod_base = gm
  modified_base 23: mod_base = cm
  modified_base 22: mod_base = gm
  modified_base 21: mod_base = tm
  modified_base 20: mod_base = gm
  modified_base 19: mod_base = tm
gcagggctccccatgtgctgtgc

SEQ ID NO: 13 - DNA, 23 bp, synthetic construct
  source 1..23: mol_type = other DNA
  modified_base 1: mod_base = tm
  modified_base 2: mod_base = cm
  modified_base 3: mod_base = OTHER; note = 2'-o-methyladenosine
  modified_base 4: mod_base = tm
  modified_base 5: mod_base = OTHER; note = 2'-o-methyladenosine
  modified_base 23: mod_base = gm
  modified_base 22: mod_base = OTHER; note = 2'-o-methyladenosine
  modified_base 21: mod_base = cm
  modified_base 20: mod_base = OTHER; note = 2'-o-methyladenosine
  modified_base 19: mod_base = cm
tcataggttgtagcacaccacag

SEQ ID NO: 14 - DNA, 23 bp, synthetic construct
  source 1..23: mol_type = other DNA
  modified_base 1: mod_base = gm
  modified_base 2..3: mod_base = cm
  modified_base 4: mod_base = gm
  modified_base 5: mod_base = tm
  modified_base 23: mod_base = tm
  modified_base 22: mod_base = cm
  modified_base 21: mod_base = tm
  modified_base 20: mod_base = tm
  modified_base 19: mod_base = cm
gccgtctcttagcagcctcttct

SEQ ID NO: 15 - DNA, 22 bp, synthetic construct
  source 1..22: mol_type = other DNA
  modified_base 1: mod_base = tm
  modified_base 2..4: mod_base = cm
  modified_base 5: mod_base = tm
  modified_base 22: mod_base = OTHER; note = 2'-o-methyladenosine
  modified_base 21: mod_base = cm
  modified_base 20: mod_base = tm
  modified_base 18..19: mod_base = cm
tcccttgggagagccttcctca

SEQ ID NO: 16 - DNA, 23 bp, synthetic construct
  source 1..23: mol_type = other DNA
  modified_base 1: mod_base = tm
  modified_base 2..4: mod_base = OTHER; note = 2'-o-methyladenosine
  modified_base 5: mod_base = gm
  modified_base 23: mod_base = OTHER; note = 2'-o-methyladenosine
  modified_base 22: mod_base = cm
  modified_base 21: mod_base = tm
  modified_base 20: mod_base = cm
  modified_base 19: mod_base = tm
taaagtagtggagagctgtctca

SEQ ID NO: 17 - DNA, 23 bp, synthetic construct
  source 1..23: mol_type = other DNA
  modified_base 1: mod_base = OTHER; note = 2'-o-methyladenosine
  modified_base 2: mod_base = cm
  modified_base 3..4: mod_base = tm
  modified_base 5: mod_base = gm
  modified_base 23: mod_base = gm
  modified_base 22: mod_base = OTHER; note = 2'-o-methyladenosine
  modified_base 21: mod_base = gm
  modified_base 20: mod_base = OTHER; note = 2'-o-methyladenosine
  modified_base 19: mod_base = tm
acttgaactcctctcccatagag

SEQ ID NO: 18 - DNA, 23 bp, synthetic construct
  source 1..23: mol_type = other DNA
tcagaccaacggcctcgttcaca

SEQ ID NO: 19 - DNA, 23 bp, synthetic construct
  source 1..23: mol_type = other DNA
caatgatggtcatgaggtaaagg

SEQ ID NO: 20 - DNA, 2373 bp, Mus musculus
  source 1..2373: mol_type = other DNA
atgcaggtccgcgtcaggctgtcgttgctgctgctctgcgcggtgctcctgggctcggcagccgcgacctcggatgacaaaactaaccaggatgactccttagattccaagagttctttgcccacagatgagtcagtgaaggaccacaccaccacgggcaaagtagttgctggccagatatttgttgattctgaagaagcagaagtggaatcccttcttcaggacgaggaagatagctccaagacccaggaggaagagatcagctttttagaatctccgaatccaagcagcaagacctacgaagaactaaagagagtgcggaagccagtcttgactgccattgaaggtacggcgcacggggagccctgccacttccctttccttttcctggataaggagtatgatgagtgcacctcagacgggagggaagatggcagactgtggtgtgccacaacctatgactacaagacagatgagaagtggggcttctgcgaaactgaagaagatgctgccaaaagacgacagatgcaggaagcagagatgatctatcaggccgggatgaagatactgaatggaagcaataggaagagccaaaagagagaagcatatcggtaccttcagaaggcagcaggcatgaatcacaccaaagccctggagagagtgtcctatgctctcttgtttggtgattacctcacacagaatatccaggcagccaaagagatgtttgagaaactgactgaggaagggtctcccaaaggacagactggtcttggctttctctacgcttctgggcttggtgttaattcaagtcaggcaaaggctcttgtatattatactttcggagctcttggaggcaacctgatagcccatatgattttgggttaccgctactgggctggcatcggagtcctccagagttgtgagtcggcactgacccattatcgtcttgttgccaatcatgttgctagtgatatctccctaactggaggctctgtagtccagagaatacggctgcccgatgaagtggaaaacccggggatgaacagtgggatgctggaagaagacctgattcagtattaccagttcctagctgagaagggtgacgtccaagcacaggttggcctgggacagctgcatctgcatggagggcgtggagtagaacagaatcaccagagagcgtttgactacttcaacttagcagcaaatgctggcaattcacatgctatggccttcctgggaaagatgtattctgaaggaagtgacatcgtacctcagagtaatgagacggcacttcactactttaagaaagctgctgacatgggcaaccccgtgggacagagcgggcttggaatggcctacctctacggaagaggcgttcaagttaattatgacctggccctcaagtatttccagaaagctgctgagcaaggctgggtggacgggcagctgcagctcggctctatgtactacaatggcattggagtcaagagagattataagcaggccttgaagtattttaatctggcttctcaaggaggccatatcttggctttctataacctcgcacagatgcacgccagcggcacaggggtgatgcggtcctgtcacactgcagtggagttgtttaagaatgtgtgtgagcgaggtcgctggtcagagagactgatgactgcctacaacagctataaggatgaggactacaatgctgcagtggtccagtacctcctgctggctgagcagggctacgaggtggcgcagagcaacgcagccttcatcctcgaccagagagaagcaaccattgtaggtgagaatgaaacttaccccagagctttactgcattggaacagggccgcctcccaaggttacactgtggctagaattaagcttggagactaccacttctatggctttggcactgatgtggattatgagaccgcatttattcattaccgcctggcttctgagcagcagcacagcgcccaagctatgtttaacctgggctacatgcacgagaagggcctaggcattaaacaggacattcaccttgcaaaacgcttttatgacatggcagccgaagctagcccagatgcacaagtacctgtgttcctcgcactctgcaaattaggtgtcgtctatttcttacagtacatacgggaagcaaacattcgagatctattcacacaactggatatggaccagcttttgggacccgagtgggacctttacctcatgaccatcattgcactgctcttgggtacagtcatagcttacaggcagcgacagcaccaggacataccagttcccaggcccccagggccacggccggctcctccccagcaggaaggaccaccagagcagcagccaccacagtag

SEQ ID NO: 21 - DNA, 2385 bp, Homo sapiens
  source 1..2385: mol_type = other DNA
atgcgggtccggatagggctgacgctgctgctgtgtgcggtgctgctgagcttggcctcggcgtcctcggatgaagaaggcagccaggatgaatccttagattccaagactactttgacatcagatgagtcagtaaaggaccatactactgcaggcagagtagttgctggtcaaatatttcttgattcagaagaatctgaattagaatcctctattcaagaagaggaagacagcctcaagagccaagagggggaaagtgtcacagaagatatcagctttctagagtctccaaatccagaaaacaaggactatgaagagccaaagaaagtacggaaaccagctttgaccgccattgaaggcacagcacatggggagccctgccacttcccttttcttttcctagataaggagtatgatgaatgtacatcagatgggagggaagatggcagactgtggtgtgctacaacctatgactacaaagcagatgaaaagtggggcttttgtgaaactgaagaagaggctgctaagagacggcagatgcaggaagcagaaatgatgtatcaaactggaatgaaaatccttaatggaagcaataagaaaagccaaaaaagagaagcatatcggtatctccaaaaggcagcaagcatgaaccataccaaagccctggagagagtgtcatatgctcttttatttggtgattacttgccacagaatatccaggcagcgagagagatgtttgagaagctgactgaggaaggctctcccaagggacagactgctcttggctttctgtatgcctctggacttggtgttaattcaagtcaggcaaaggctcttgtatattatacatttggagctcttgggggcaatctaatagcccacatggttttgggttacagatactgggctggcatcggcgtcctccagagttgtgaatctgccctgactcactatcgtcttgttgccaatcatgttgctagtgatatctcgctaacaggaggctcagtagtacagagaatacggctgcctgatgaagtggaaaatccaggaatgaacagtggaatgctagaagaagatttgattcaatattaccagttcctagctgaaaaaggtgatgtacaagcacaggttggtcttggacaactgcacctgcacggagggcgtggagtagaacagaatcatcagagagcatttgactacttcaatttagcagcaaatgctggcaattcacatgccatggcctttttgggaaagatgtattcggaaggaagtgacattgtacctcagagtaatgagacagctctccactactttaagaaagctgctgacatgggcaacccagttggacagagtgggcttggaatggcctacctctatgggagaggagttcaagttaattatgatctagcccttaagtatttccagaaagctgctgaacaaggctgggtggatgggcagctacagcttggttccatgtactataatggcattggagtcaagagagattataaacaggccttgaagtattttaatttagcttctcagggaggccatatcttggctttctataacctagctcagatgcatgccagtggcaccggcgtgatgcgatcatgtcacactgcagtggagttgtttaagaatgtatgtgaacgaggccgttggtctgaaaggcttatgactgcctataacagctataaagatggcgattacaatgctgcagtgatccagtacctcctcctggctgaacagggctatgaagtggcacaaagcaatgcagcctttattcttgatcagagagaagcaagcattgtaggtgagaatgaaacttatcccagagctttgctacattggaacagggccgcctctcaaggctatactgtggctagaattaagctcggagactaccatttctatgggtttggcaccgatgtagattatgaaactgcatttattcattaccgtctggcttctgagcagcaacacagtgcacaagctatgtttaatctgggatatatgcatgagaaaggactgggcattaaacaggatattcaccttgcgaaacgtttttatgacatggcagctgaagccagcccagatgcacaagttccagtcttcctagccctctgcaaattgggcgtcgtctatttcttgcagtacatacgggaaacaaacattcgagatatgttcacccaacttgatatggaccagcttttgggacctgagtgggacctttacctcatgaccatcattgcgctgctgttgggaacagtcatagcttacaggcaaaggcagcaccaagacatgcctgcacccaggcctccagggccacggccagctccaccccagcaggaggggccaccagagcagcagccaccacagtaa

SEQ ID NO: 22 - RNA, 20 nt, synthetic construct
  source 1..20: mol_type = other RNA
atgagtgcacctcagacggg

SEQ ID NO: 23 - RNA, 20 nt, synthetic construct
  source 1..20: mol_type = other RNA
aagtgcgtattgttcaagtg

SEQ ID NO: 24 - DNA, 200 bp, synthetic construct
  source 1..200: mol_type = other DNA
cacggggagccctgccacttccctttccttttcctggataaggagtatgatgagtacacatccgatggaagagaagatggcagactgtggtgtgccacaacctatgactacaagacagatgagaagtggggcttctgcgaaagtgcgtattgttcaagtgcacgccctgtgctttagggcagcatttggaaggcattttc

SEQ ID NO: 25 - DNA, 24 bp, synthetic construct
  source 1..24: mol_type = other DNA
agtacacatccgatggaagagaag

SEQ ID NO: 26 - DNA, 22 bp, synthetic construct
  source 1..22: mol_type = other DNA
gaaaatgccttccaaatgctgc

SEQ ID NO: 27 - DNA, 21 bp, synthetic construct
  source 1..21: mol_type = other DNA
agtgcacctcagacgggaggg

SEQ ID NO: 28 - DNA, 22 bp, synthetic construct
  source 1..22: mol_type = other DNA
gaaaatgccttccaaatgctgc

SEQ ID NO: 29 - DNA, 29 bp, synthetic construct
  source 1..29: mol_type = other DNA
cttaagaactcaaagtctacactaagtct

SEQ ID NO: 30 - DNA, 25 bp, synthetic construct
  source 1..25: mol_type = other DNA
cagcttgcctcaagggtttacagaa

SEQ ID NO: 31 - DNA, 25 bp, synthetic construct
  source 1..25: mol_type = other DNA
ccattgaaggcacggcgcacgggga

SEQ ID NO: 32 - DNA, 27 bp, synthetic construct
  source 1..27: mol_type = other DNA
gcgccgtgccttcaatggcagtcaaga

SEQ ID NO: 33 - DNA, 31 bp, synthetic construct
  source 1..31: mol_type = other DNA
cttctgcgaaaggcgtattgttcaagtgggg

SEQ ID NO: 34 - DNA, 32 bp, synthetic construct
  source 1..32: mol_type = other DNA
aacaatacgcctttcgcagaagccccacttct

SEQ ID NO: 35 - DNA, 23 bp, synthetic construct
  source 1..23: mol_type = other DNA
gtatgatgagtacacctcagacg

SEQ ID NO: 36 - DNA, 23 bp, synthetic construct
  source 1..23: mol_type = other DNA
cgtctgaggtgtactcatcatac

SEQ ID NO: 37 - DNA, 32 bp, synthetic construct
  source 1..32: mol_type = other DNA
atgcaggtccgcgtcaggctgtcgttgctgct

SEQ ID NO: 38 - DNA, 24 bp, synthetic construct
  source 1..24: mol_type = other DNA
ctactgtggtggctgctgctctgg

SEQ ID NO: 39 - DNA, 29 bp, synthetic construct
  source 1..29: mol_type = other DNA
gcggctagcatgcgggtccggatagggct

SEQ ID NO: 40 - DNA, 32 bp, synthetic construct
  source 1..32: mol_type = other DNA
gcgaagcttttactgtggtggctgctgctctg

SEQ ID NO: 41 - DNA, 20 bp, synthetic construct
  source 1..20: mol_type = other DNA
agcaagacctacgaagaact

SEQ ID NO: 42 - DNA, 24 bp, synthetic construct
  source 1..24: mol_type = other DNA
gaaggtaccgatatgcttctctct

SEQ ID NO: 43 - DNA, 32 bp, synthetic construct; identical to SEQ ID NO: 37
  source 1..32: mol_type = other DNA

SEQ ID NO: 44 - DNA, 24 bp, synthetic construct
  source 1..24: mol_type = other DNA
ctactgtggtggctgctgctctgg

SEQ ID NO: 45 - DNA, 18 bp, synthetic construct
  source 1..18: mol_type = other DNA
tggtgagcaagggcgagg

SEQ ID NO: 46 - DNA, 22 bp, synthetic construct
  source 1..22: mol_type = other DNA
cgtccttgaagaagatggtgcg

SEQ ID NO: 47 - RNA, 23 nt, synthetic construct
  source 1..23: mol_type = other RNA
gcgagtcttgactgccattgaag

SEQ ID NO: 48 - DNA, 22 bp, synthetic construct
  source 1..22: mol_type = other DNA
cgtccttgaagaagatggtgcg

SEQ ID NO: 49 - DNA, 26 bp, synthetic construct
  source 1..26: mol_type = other DNA
ggggaaagtgtcacagaagatatcag

SEQ ID NO: 50 - DNA, 21 bp, synthetic construct
  source 1..21: mol_type = other DNA
gacactctctccagggctttg

SEQ ID NO: 51 - DNA, 20 bp, synthetic construct
  source 1..20: mol_type = other DNA
acgaggttccagaggtggag

SEQ ID NO: 52 - DNA, 20 bp, synthetic construct
  source 1..20: mol_type = other DNA
aagaggcaacagtgtcagag

SEQ ID NO: 53 - DNA, 20 bp, synthetic construct
  source 1..20: mol_type = other DNA
gaatgaagtgaggccagtgg

SEQ ID NO: 54 - DNA, 20 bp, synthetic construct
  source 1..20: mol_type = other DNA
actgggtccttctgggtaga

SEQ ID NO: 55 - DNA, 21 bp, synthetic construct
  source 1..21: mol_type = other DNA
gagcaacaagaaaaccaagca

SEQ ID NO: 56 - DNA, 21 bp, synthetic construct
  source 1..21: mol_type = other DNA
tgcacacaagccatctactca

SEQ ID NO: 57 - DNA, 22 bp, synthetic construct
  source 1..22: mol_type = other DNA
ggcaaatgctggacccaacaca

SEQ ID NO: 58 - DNA, 22 bp, synthetic construct
  source 1..22: mol_type = other DNA
tgctggtcttgccattcctgga

SEQ ID NO: 59 - DNA, 23 bp, Mus musculus
  source 1..23: mol_type = genomic DNA
agtgcacctcagacgggagggaa

SEQ ID NO: 60 - DNA, 23 bp, synthetic construct
  source 1..23: mol_type = other DNA
agtacacatccgatggaagagaa

SEQ ID NO: 61 - DNA, 40 bp, synthetic construct
  source 1..40: mol_type = other DNA
agccagtcttgactgccattgaaggtacggcgcacgggga

SEQ ID NO: 62 - DNA, 14 bp, synthetic construct
  source 1..14: mol_type = other DNA
cgaaactgaagaag

SEQ ID NO: 63 - DNA, 24 bp, synthetic construct
  source 1..24: mol_type = other DNA
agccagtcttgactgccattgaag

SEQ ID NO: 64 - skipped

SEQ ID NO: 65 - DNA, 15 bp, Mus musculus
  source 1..15: mol_type = genomic DNA
gcgaaagtgcgtatt

SEQ ID NO: 66 - DNA, 15 bp, synthetic construct
  source 1..15: mol_type = other DNA
gcgaaagtgcgtatt

SEQ ID NO: 67 - DNA, 14 bp, synthetic construct
  source 1..14: mol_type = other DNA
gcgaaaggcgtatt

SEQ ID NO: 68 - DNA, 23 bp, Mus musculus
  source 1..23: mol_type = genomic DNA
agtgcacctcagacgggagggaa

SEQ ID NO: 69 - DNA, 23 bp, synthetic construct
  source 1..23: mol_type = other DNA
agtacacatccgatggaagagaa

SEQ ID NO: 70 - DNA, 20 bp, Mus musculus
  source 1..20: mol_type = genomic DNA
ccattgaaggtacggcgcac

SEQ ID NO: 71 - DNA, 20 bp, Rattus norvegicus
  source 1..20: mol_type = genomic DNA
ccattgaaggtacggcgcac

SEQ ID NO: 72 - DNA, 20 bp, Homo sapiens
  source 1..20: mol_type = genomic DNA
ccattgaaggcacagcacat

SEQ ID NO: 73 - DNA, 20 bp, Pan troglodytes
  source 1..20: mol_type = genomic DNA
ccattgaaggcacagcacat

SEQ ID NO: 74 - DNA, 20 bp, Canis lupus
  source 1..20: mol_type = genomic DNA
ccattgaaggcacagcacat

SEQ ID NO: 75 - DNA, 20 bp, Gallus gallus
  source 1..20: mol_type = genomic DNA
ctattggaggcactgcagat

SEQ ID NO: 76 - DNA, 20 bp, Danio rerio
  source 1..20: mol_type = genomic DNA
tgaatggtggaacggcctct

SEQ ID NO: 77 - protein, 50 aa, Homo sapiens
AHGEPCHFPFPLFLDKEYDECTSDGREDGRLWCATTYDYKADEKWGFCET

SEQ ID NO: 78 - protein, 49 aa, Pan troglodytes
AHGEPCHFPFLFLDKEYDECTSDGREDGRLWCATTYDYKADEKWGFCET

SEQ ID NO: 79 - protein, 50 aa, Canis lupus
AHGEPCHFPOFLFLDKEYDECTSDGREDGRLWCATTYDYKADEKWGFCET

SEQ ID NO: 80 - protein, 50 aa, Mus musculus
AHGEPCHFPFLFLDKEYDECTSDGREDGRELWCATTYDYKTDEKWGFCET

SEQ ID NO: 81 - protein, 49 aa, Rattus norvegicus
AHGEPCHFPFLFLDKEYDECTSDGREDGRLWCATTYKYKTDEKWGFCET

SEQ ID NO: 82 - protein, 49 aa, Gallus gallus
ADGEPCHFPFLFMEKEYAECTADGREDGRLWCATTYDYKKDQKWGFCET

SEQ ID NO: 83 - protein, 49 aa, Danio rerio
ASGEPCIFPFLFQGKEYSDCTTEGRGDGRLWCATTYDYNNDKRWGFCET

SEQ ID NO: 84 - DNA, 36 bp, Mus musculus
  source 1..36: mol_type = genomic DNA
tatgatgagtgcacctcagacgggagggaagatggc

SEQ ID NO: 85 - DNA, 26 bp, Mus musculus
  source 1..26: mol_type = genomic DNA
gaaagtgcgtattgttcaagtggggc

SEQ ID NO: 86 - protein, 13 aa, Mus musculus
YDECTSDGREDGE

SEQ ID NO: 87 - DNA, 36 bp, Mus musculus
  source 1..36: mol_type = genomic DNA
tatgatgagtacacatccgatggaagagaagatggc

SEQ ID NO: 88 - DNA, 23 bp, Mus musculus
  source 1..23: mol_type = genomic DNA
gaaagtgcgtattgttcaagtgc

SEQ ID NO: 89 - protein, 12 aa, Mus musculus
YDEYTSDGEDGE

SEQ ID NO: 90 - DNA, 42 bp, Mus musculus
  source 1..42: mol_type = other DNA
aaggagtatgatgagtgcacctcagacgggagggaagatggc

SEQ ID NO: 91 - protein, 14 aa, Mus musculus
KEYDECTSDGREDG

SEQ ID NO: 92 - DNA, 41 bp, synthetic construct
  source 1..41: mol_type = other DNA
aaggagtatgatgagtrcacmtcmganggrgrgaagatggc

SEQ ID NO: 93 - protein, 14 aa, synthetic construct
  SITE 6: note = X is C or Y
KEYDEXTSDGREDG

SEQ ID NO: 94 - DNA, 42 bp, Synthetic construct
  source 1..42: mol_type = other DNA
aaggagtatgatgagtacacatccgatggaagagaagatggc

SEQ ID NO: 95 - protein, 14 aa, synthetic construct
KEYDEYTSDGREDG

SEQ ID NO: 96 - DNA, 31 bp, unidentified
  source 1..31: mol_type = genomic DNA
gggcgagtcttgactgccattgaaggtacgg

SEQ ID NO: 97 - DNA, 15 bp, unidentified
  source 1..15: mol_type = genomic DNA
gcgaaaggcgatgcc

SEQ ID NO: 98 - DNA, 25 bp, unidentified
  source 1..25: mol_type = genomic DNA
gggcgagtcttgactgccattgaag

SEQ ID NO: 99 - DNA, 20 bp, unidentified
  source 1..20: mol_type = other DNA
ggaaaccagctttgaccgcc

SEQ ID NO: 100 - DNA, 23 bp, unidentified
  source 1..23: mol_type = other DNA
tcttttcctagataaggagtatg

SEQ ID NO: 101 - DNA, 20 bp, unidentified
  source 1..20: mol_type = other DNA
ttgtgaaactgaagaagagg

SEQ ID NO: 102 - DNA, 12 bp, synthetic construct
  source 1..12: mol_type = other DNA
ctgaagaagagg

SEQ ID NO: 103 - DNA, 12 bp, unidentified
  source 1..12: mol_type = other DNA
ataaggagtatg

SEQ ID NO: 104 - DNA, 20 bp, unidentified
  source 1..20: mol_type = other DNA
ttgtgaaactgaagaagagg

SEQ ID NO: 105 - DNA, 25 bp, synthetic construct
  source 1..25: mol_type = other DNA
cctcttacctcagttacaatttata

SEQ ID NO: 106 - DNA, 42 bp, synthetic construct
  source 1..42: mol_type = other DNA
gatgagtgcacatccgatggaagagaagatggcagactgtgg

SEQ ID NO: 107 - DNA, 40 bp, synthetic construct
  source 1..40: mol_type = other DNA
gccatcttctcttccatcggatgtgcactcatcatactcc

(19) United States
(12) Patent Application Publication
(10) Pub. No.: US 2026/0159834 A1
(43) Pub. Date: Jun. 11, 2026

(54) COMPOSITIONS, METHODS, AND KITS RELATING TO THERAPEUTICS FOR THE MODULATION OF SEL1L-HRD1 ERAD ACTIVITY AND FUNCTION

(71) Applicant: University of Virginia Patent Foundation, Charlottesville, VA (US)

(72) Inventors: Ling Qi, Keswick, VA (US); Helen Wang, Charlottesville, VA (US); Shengyi Iris Sun, Keswick, VA (US)

(21) Appl. No.: 19/410,278

(22) Filed: Dec. 5, 2025

Related U.S. Application Data
(60) Provisional application No. 63/728,512, filed on Dec. 5, 2024.

(51) Int. Cl. C12N 15/113; A61P 37/00; C12N 15/86

(52) U.S. Cl. CPC C12N 15/113; A61P 37/00; C12N 15/86; C12N 2310/11; C12N 2310/20; C12N 2310/341; C12N 2750/14143

(57) ABSTRACT

Described herein are compositions, methods, and kits related to therapeutic modulation of the SEL1L-HRD1 ERAD pathway based on demonstrated correction of disease phenotypes in mouse models and patient-derived cells. Antisense oligonucleotide (ASO)-induced skipping of SEL1L exon 4 can rescue ERAD deficiency and multiple physiological defects in mammals and can restore ERAD function in patient cells.

### CROSS-REFERENCE TO RELATED APPLICATION[S]

This application claims priority to, and the benefit of, U.S. Provisional Patent Application Ser. No. 63/728,512, entitled “Regulation of SEL1L-HRD1 ERAD activity and function using ASO in health and disease” and filed on Dec. 5, 2024, the entire contents of which are incorporated herein by reference as if set forth in its entirety.

### STATEMENT REGARDING FEDERALLY SPONSORED RESEARCH OR DEVELOPMENT

This invention was made with Government support under contract R01AG089640 awarded by the National Institutes of Health National Institute on Aging (NIA) and R35GM130292 awarded by the National Institutes of Health National Institute of General Medical Sciences (NIGMS). The Government has certain rights in the invention.

### SEQUENCE LISTING

The instant application contains a Sequence Listing which has been submitted electronically in .XML format and is hereby incorporated by reference in its entirety. Said .XML copy, created on Dec. 5, 2025, is named “222117_1650_Sequence_Listing.xml” and is 136,978 bytes in size.

### BACKGROUND

The SEL1L adaptor subunit of SYVN1 ubiquitin ligase-ERAD-associated E3 ubiquitin-protein ligase HRD1 (SEL1L-HRD1) endoplasmic reticulum-associated degradation (ERAD) pathway is essential for maintaining protein homeostasis (1-3). Genetic variants and mutations that impair SEL1L-HRD1 function cause severe congenital disorders, including ERAD-associated neurodevelopmental disorder (ENDI) and ENDI-Agammaglobulinemia (ENDI-A) (4, 5). ENDI patients presented invariably intellectual disability, developmental delay, short stature, underweight, facial dysmorphisms and locomotor dysfunctions (4). A specific severe form of ENDI, ENDI-A patients presented additional symptoms, including frequent infections, agammaglobulinemia, B cell lymphopenia, and early death (5). Consistently, mouse carrying biallelic pathogenic SEL1L mutations (SEL1L S658P mutation and C141Y mutation) also exhibited similar phenotypes (6), including perinatal lethality, developmental delay, ataxia, and immune deficiency. There is a need to address the aforementioned deficiencies accordingly.

### SUMMARY

Described herein are compositions, pharmaceutical compositions, methods, and kits relating to therapeutics for the modulations of SEL1L adaptor subunit of SYVN1 ubiquitin ligase-ERAD-associated E3 ubiquitin-protein ligase HRD1 (SEL1L-HRD1) endoplasmic reticulum-associated degradation (ERAD) pathway activity and function.

Described herein are compositions. In embodiments, described herein are one or more nucleotide-based therapeutics for SEL1L adaptor subunit of SYVN1 ubiquitin ligase-ERAD-associated E3 ubiquitin-protein ligase HRD1 (SEL1L-HRD1) endoplasmic reticulum-associated degradation (ERAD) pathway (SEL1L-HRD1 ERAD pathway) modulation. In embodiments, the one or more nucleotide-based therapeutics comprise one or more antisense oligonucleotides (ASOs), one or more gapmer ASOs, one or more CRISPR interference constructs, or one or more nucleotide-based therapeutics that increase the expression of SEL1L, individually or in any combination of any thereof. In embodiments, the one or more nucleotide-based therapeutics target a SEL1L pre-mRNA or mRNA.

In embodiments, the one or more ASOs comprise one or more nucleotides having at least 85% sequence identify to any one or more of SEQ ID NOs: 1-7.

In embodiments, the one or more gapmer ASOs comprise one or more nucleotides having at least 85% sequence identify to any one or more of SEQ ID NOs: 8-19.

In embodiments, the one or more CRISPR interference constructs comprise one or more nucleotides having at least 85% sequence identify to any one or more of SEQ ID NOs: 22-23.

In embodiments, the one or more nucleotide-based therapeutics that increase the expression of SEL1L comprise an SEL1L sequence or active fragment thereof having at least 85% sequence identify to SEQ ID NOs: 20 or 21.

Described herein are vectors (i.e., expression vectors). In embodiments, described herein are vectors comprising a polynucleotide that encode[s] one or more nucleotide-based therapeutics described herein. In embodiments, the vector is an adeno-associated viral (AAV) vector. In embodiments, the AAV vector is an AAV-PHP.eB-eGFP vector comprising an SEL1L sequence or active fragment thereof having at least 85% sequence identify to SEQ ID NOs: 20 or 21.

Described herein are pharmaceutical compositions. In embodiments, pharmaceutical compositions described herein comprise one or more compositions, one or more nucleotide-based therapeutics, or one or more vectors described herein and a pharmaceutically-acceptable carrier.

Described herein are methods of modulating SEL1L splicing or SEL1L expression. In embodiments, methods of modulating SEL1L splicing or SEL1L expression can comprise administering one or more nucleotide-based therapeutics described herein to a subject in need thereof. In embodiments of methods of modulating SEL1L splicing, one or more ASOs can be administered to a subject in need thereof in an amount effective to modulate splicing of SEL1L mRNA or increase SEL1L expression. In embodiments, the subject in need thereof is a mammal, cell, or tissue, having or suspected of having one or more SEL1L mutations. In embodiments, the one or more SEL1L mutations are in exon 4 of SEL1L of the subject. In embodiments, the one or more SEL1L mutations comprise one or more of C141Y or S658P. In embodiments, the one or more nucleotide-based therapeutics are administered in an amount affective to improve any one or more of body weight, neurological symptoms, any one or more symptoms of ERAD deficiency, perinatal lethality, or B-cell lymphopenia.

Described herein are methods of reducing one or more symptoms (or otherwise correcting, improving, or normalizing one or more symptoms to a level of a subject not having an ERAD-dysfunction related disease) in a subject in need thereof. In embodiments, methods comprise administering one or more nucleotide-based therapeutics, vectors, or pharmaceutical compositions to a subject in need thereof. In embodiments, the subject in need thereof has or is suspected of having ENDI-agammaglobulinemia (ENDI-A). In embodiments, the one or more nucleotide-based therapeutics target a SEL1L pre-mRNA or mRNA. In embodiments, the subject in need thereof has one or more SEL1L mutations in exon 4 of SEL1L. In embodiments, the one or more SEL1L mutations comprise one or more of C141Y or S658P. In embodiments, the one or more nucleotide-based therapeutics can be administered in an amount affective to improve any one or more of: body weight, neurological symptoms, symptoms of developmental delay, intellectual disability, B cell lymphopenia and agammaglobuinemia, or axial hypotonia, individually or in any combination of any thereof.

In embodiments described herein are compositions comprising one or more nucleotide-based therapeutics targeting a nucleotide sequence related to SEL1L.

In embodiments, the one or more nucleotide-based therapeutics targeting a nucleotide sequence related to SEL1L comprise one or more antisense oligonucleotides (ASOs or gapmer ASOs. In embodiments, the sequence related to SEL1L is a SEL1L pre-mRNA or mRNA.

In embodiments, the one or more ASOs comprise one or more nucleotides having at least 85% sequence identify to any one or more of SEQ ID NOs: 1-7.

In embodiments, the one or more gapmer ASOs comprise one or more nucleotides having at least 85% sequence identify to any one or more of SEQ ID NOs: 8-19.

In embodiments described herein are compositions comprising one or more nucleotide-based therapeutics that can inhibit the expression or activity or SEL1L. In embodiments, the one or more nucleotide-based therapeutics that can inhibit SEL1L expression or activity comprise one or more CRISPR interference constructs.

In embodiments, the one or more CRISPR interference constructs comprise one or more nucleotides having at least 85% sequence identify to any one or more of SEQ ID NOs: 22-23.

In embodiments described herein are compositions comprising one or more nucleotide-based therapeutics that can increase the expression of SEL1L. In embodiments, the one or more nucleotide-based therapeutics that increase the expression of SEL1L comprise an adeno-associated viral (AAV) vector encoding an SEL1L sequence or active fragment thereof. In embodiments, the adeno-associated viral (AAV) vector encoding an SEL1L sequence or active fragment thereof comprises an SEL1L sequence or active fragment thereof having at least 85% sequence identify to any one or more of SEQ ID NOs: 20 or 21. In embodiments, the AAV vector is an AAV-PHP.eB-eGFP vector.

Described herein are vectors, comprising a polynucleotide that encodes one or more nucleotide-based therapeutics described herein.

Described herein are pharmaceutical compositions, comprising one or more nucleotide-based therapeutics described herein, compositions comprising one or more nucleotide-based therapeutics described herein, or vectors described herein, and one or more pharmaceutically-acceptable carriers.

Described herein are methods of modulating SEL1L splicing. In embodiments, methods of splice modulation can comprise administering one or more nucleotide-based therapeutics targeting a nucleotide sequence related to SEL1L to a subject in need thereof. In embodiments, the sequence related to SEL1L is a SEL1L pre-mRNA or mRNA. In embodiments, the one or more nucleotide-based therapeutics can be one or more ASOs having at least 85% sequence identify to any one or more of SEQ ID NOs: 1-7. In embodiments, the one or more nucleotide-based therapeutics can be one or more gapmer ASOs having at least 85% sequence identify to any one or more of SEQ ID NOs: 8-19. In embodiments, the ASOs and/or gapmer ASOs can be administered in an amount effective to modulate splicing of SEL1L mRNA. In embodiments, the subject in need thereof is a mammal, cell, or tissue, having or suspected of having one or more SEL1L mutations. In embodiments, the one or more SEL1L mutations can be in exon 4 of SEL1L of the subject. In embodiments, the one or more SEL1L mutations comprise one or more of C141Y or S658P. In embodiments, the one or more ASOs and/or gapmer ASOs are administered in an amount affective to improve, in the subject, any one or more of body weight, neurological symptoms, any one or more symptoms of ERAD deficiency, perinatal lethality, or B-cell lymphopenia. In embodiments, the subject in need thereof can have or be suspected of having ENDI-A syndrome (which can be specific for mutations in the SEL1L exon 4). ENDI-A syndrome can include a group of symptoms of developmental delay, intellectual disability, B cell lymphopenia agammaglobulinemia, and/or axial hypotonia, and the one or more ASOs and/or gapmer ASOs can be administered to a subject in an amount effect to improve (such as improve symptoms relating to intellectual disability), reduce (as in reduce intellectual disability and/or developmental delay overall), or otherwise normalize (i.e., return to a non-diseased state or a state in a subject where any deficit may not be recognizable, for example, recognizable without a detailed clinical exam) any one or more symptoms of ENDI-A syndrome. In embodiments, the ASOs and/or gapmer ASOs can be administered in an amount effective to modulate splicing of SEL1L.

Described herein are methods of inhibiting SEL1L. In embodiments, methods of inhibiting SEL1L can comprise administering one or more CRISPR interference constructs to a subject in need thereof or other inhibiting constructs. In embodiments, the subject in need thereof can be a subject having or suspected of having Alzheimer's disease. In embodiments, the one or more interference constructs can be administered in an amount effective to reduce one or more symptoms of Alzheimer's disease.

Described herein are methods of increasing SEL1L expression. In certain aspects, the subject in need thereof can be a subject having or suspected of having ENDI syndrome, characterized by a group of symptoms that include, for example: developmental delay, intellectual disability, locomotor dysfunction, and/or facial dysmorphisms.

In embodiments of methods described herein, a subject in need thereof is a mammal, cell, or tissue, having or suspected of having one or more SEL1L mutations. In embodiments, the one or more SEL1L mutations are in exon 4 of SEL1L of the subject. In embodiments, the one or more SEL1L mutations comprise one or more of C141Y or S658P. In embodiments, a subject in need thereof is a subject having or suspected of having ENDI syndrome (in particular, ENDI-A). In embodiments, the one or more nucleotide-based therapeutics are administered in an amount effective to improve any one or more of body weight or neurological symptoms, or any one or more symptoms of ERAD deficiency, perinatal lethality, or B-cell lymphopenia, individually or in any combination of any thereof.

Described herein are methods of reducing one or more symptoms of an ERAD dysfunction-related disease. In embodiments, a method of reducing one or more symptoms of an ERAD dysfunction-related disease can comprise: administering one or more nucleotide-based therapeutics targeting a nucleotide sequence related to SEL1L or that increase the expression of SEL1L to a subject in need thereof. In embodiments, the one or more nucleotide-based therapeutics targeting a nucleotide sequence related to SEL1L can comprise one or more antisense oligonucleotides (ASOs), one or more gapmer ASOs, or one or more CRISPR interference constructs. In embodiments of methods described herein, the one or more nucleotide-based therapeutics that increase the expression of SEL1L can comprise an adeno-associated viral (AAV) vector encoding an SEL1L sequence or active fragment thereof. In embodiments, the one or more nucleotide-based therapeutics can be administered in an amount affective to improve any one or more of body weight or neurological symptoms, or any one or more symptoms of ERAD deficiency, perinatal lethality, or B-cell lymphopenia. In embodiments, the subject in need thereof is a subject having or suspected of having ENDI.

Described herein are kits. In embodiments, a kit comprises one or more nucleotide-based therapeutics for SEL1L adaptor subunit of SYVN1 ubiquitin ligase-ERAD-associated E3 ubiquitin-protein ligase HRD1 (SEL1L-HRD1) endoplasmic reticulum-associated degradation (ERAD) pathway (SEL1L-HRD1 ERAD pathway) modulation and instructions for use.

In embodiments, a kit comprises one or more nucleotide-based therapeutics targeting a nucleotide sequence related to SEL1L or that can increase the expression of SEL1L, or one or more nucleotide-based therapeutics that can inhibit or otherwise reduce SEL1L activity. In embodiments, the one or more nucleotide-based therapeutics targeting a nucleotide sequence related to SEL1L comprise one or more antisense oligonucleotides (ASOs), one or more gapmer ASOs, one or more CRISPR interference constructs, or one or more nucleotide-based therapeutics that increase the expression of SEL1L (for example, one that comprises an adeno-associated viral (AAV) vector encoding an SEL1L sequence or active fragment thereof) and instructions for use. Kits can comprise any one or more nucleotides, compositions, pharmaceutical compositions, or vectors according to the present disclosure. In embodiments, any of the above can be provided in a dosage-unit form in the kit.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles.

Moreover, in the drawings, like reference numerals designate corresponding parts throughout the several views.

FIGS. 1A-1I. The alternative splicing of Sel1L exon 4 rescues ERAD function and lethality of SEL1L C141Y knock-in (KI) mice. (FIG. 1A) A diagram showing the location of SEL1L C141Y mutation (red line) and Fibronectin II (FNII) domain on SEL1L gene. (FIG. 1B) Sanger sequencing of Sel1L genes from the homozygous progenies of WT and KI founder lines A/B/C. Red shaded box highlights the mutation identified in exon 4. (FIG. 1C) The survival rate of KI/KI′ pups and their WT and HET littermates at postnatal day 21 (P21). (FIG. 1D) The diagram of the Sel1L isoforms and the primer design for (FIG. 1E) and (FIG. 1G). (FIG. 1E) DNA agarose gel electrophoresis analysis and quantitation of full length SEL1L transcript from WT human livers and Sel1L transcripts from WT, KI and KI′ mouse livers at ages of postnatal day 0 (P0) and 1 month (1 m), respectively. PPIA and L32, internal controls for human and mouse tissues, respectively. n=3-4 mice/group. (FIG. 1F) Sanger sequencing of SEL1L cDNA, focusing on exon 4 and splicing junctions. Red arrow and shaded red box, the alternative GT donor. (FIG. 1G) DNA polyacrylamide gel electrophoresis (PAGE) analysis and quantitation of Sel1L exon 4 isoforms in mouse livers at indicated ages. n=3-4 mice/group. (FIG. 1H) Western blot analysis of ERAD proteins and known ERAD substrate IRE1α in P0 mouse livers. Two SEL1L antibodies (AB1, AB2) were used as shown FIG. 8F. (FIG. 1I) Quantitation of western blot analysis of ERAD proteins and substrates in mouse livers. SEL1L from both antibodies were quantitated as average. Sex and age combined. n=5-17 mice/group. Asterisks, non-specific bands. Data are represented as means±SEM. n.s., not significant. *p<0.05; **p<0.01; ***p<0.001, ****p<0.0001, Chi-square test for (C); Two-way ANOVA followed by Tukey's multiple-comparisons test for (FIG. 1E and FIG. 1G); One-way ANOVA followed by Tukey's post hoc test for (FIG. 1I).

FIGS. 2A-2H. The mutation of major splicing donor enables the usage of an internal alternative splice donor. (FIG. 2A) Sanger sequencing results of SEL1L exon 4-intron 4 junction. Mutation was indicated by the red shaded box. (FIG. 2B) Schematic of the alternative splicing of Sel1L exon 4 with the percentage of isoforms in KI and KI′ mice indicated on the left. (FIG. 2C) Diagram of the splicing minigene reporter. (FIG. 2D and FIG. 2G) Diagrams of the indicated mutations in the minigene reporter construct, including primer design and the expected splicing products. (FIGS. 2E and H) DNA PAGE analysis of the WT and mutated minigene reporters as indicated by using primer pair F4A/R4. (FIG. 2F) Quantitation of (FIG. 2E) and (FIG. 2H) as percent of exon 4 inclusion. n=3 independent biological replicates. Data are represented as means±SEM. n.s., not significant. *p<0.05; **p<0.01; ***p<0.001, ****p<0.0001, Two-way ANOVA followed by Tukey's multiple-comparisons test for (FIG. 2F).

FIGS. 3A-3K. The alternative splicing of Sel1L exon 4 rescues developmental defects and B cell lymphopenia in SEL1L C141Y KI mice. (FIG. 3A) The survival rate of WT, HET and KI pups at postnatal day 0 (P0). (FIG. 3B) Body weight at P0. n=7-9 mice/group. (FIG. 3C) The photo of pups at postnatal 4 hours (upper) and >12 hours (lower). (FIG. 3D) The survival curve of WT, HET and KI pups after birth. (FIG. 3E) Body weight growth of male WT, HET, KI′ and surviving KI mice for postnatal week 1 to week 10. (FIG. 3F) Quantification of B cells as a percentage of CD45⁺ peripheral blood mononuclear cells (PBMCs) at P0 by flow cytometry in FIG. 11B to FIG. 11C. n=5-10 mice/group. (FIG. 3G) Immunofluorescence of CD19+ B cells in P0 mouse spleen with quantitation shown in FIG. 10F. Red, CD19; Blue, DAPI. n=3 mice/group. (FIG. 3H) Percentage of lymphocytes in the peripheral blood mononuclear cells (PBMCs) of WT, KI′ and surviving KI mice. Age and sex combined. n=4-11 mice/group. (1) Western blot analysis of SEL1L protein expression in mouse brains or cortex at indicated ages with quantitation in FIG. 12B and FIG. 12D. (FIG. 3J and FIG. 3K) Immunofluorescence (FIG. 3J) and quantitation (FIG. 3K) of SATB2+ Layer II/Ill cells number below the CTIP2+ Layer V in the cortex of p0 WT, KI and KI′ mice, the region as indicated by the Zoom in box. n=3-6 mice/group. Green, CTIP2; Red, SATB2; Blue, DAPI. Data are represented as means±SEM. n.s., not significant. *p<0.05; **p<0.01; ***p<0.001, ****p<0.0001, Chi-square test for (FIG. 3A); Two-tailed t test for (FIG. 3B); Mantel-Cox test for (FIG. 3D); Two-way ANOVA followed by Tukey's multiple-comparisons test for (FIG. 3E); One-way ANOVA followed by Tukey's post hoc test for (FIG. 3F, FIG. 3H, FIG. 3K).

FIGS. 4A-4J. ASO-mediated exon skipping rescues ERAD defects in C141Y patient-derived fibroblasts. (FIG. 4A) Sequence alignment of the alternative splice site within Sel1L exon 4 across different species. (FIG. 4B) The experimental design of the ASOs screen targeting SEL1L Exon 4. (FIG. 4C and FIG. 4D) DNA PAGE analysis (FIG. 4C) and quantitation (FIG. 4D) of ASOs treatment at 10 μM for 3 days in C141Y patient fibroblasts, with a diagram showing corresponding products. C, Control ASO. Red arrowhead indicates the location of SEL1L C141Y mutation. n=5 independent biological replicates. Statistics indicates the comparison between control ASO and ASO 1-7 targeting SEL1L exon 4 and introns 3 and 4. (FIG. 4E) DNA agarose gel electrophoresis analysis of ASO1 treatment at 10 μM for 3 days in human WT and C141Y patient fibroblasts. Quantitation shown in the FIG. 15A. n=5-6 independent biological replicates. (FIG. 4F to FIG. 4H) Western blot analysis (FIG. 4F) and quantitation (FIG. 4G and FIG. 4H) of ASO1 treatment at 10 μM for 3 days in human WT and C141Y patient fibroblasts. Two SEL1L antibodies (AB1, AB2) were used as shown FIG. 8F. n=3-4 independent biological replicates. (FIG. 4I) Electrophoresis analysis of XBP1 splicing of ASO1 treatment at 10 μM for 3 days in human WT and C141Y patient fibroblasts, with quantitation below. DNA PAGE gel for the XBP1 splicing and agarose gel for the internal control PPIA. n=3 independent replicates for each group. n=1 for positive controls. u, unspliced. s, spliced. (FIG. 4J) Schematic model illustrating rescue of the SEL1L C141Y mutation through alternative splicing modulation in mice and humans. Data are represented as means±SEM. n.s., not significant. *p<0.05; **p<0.01; ***p<0.001, ****p<0.0001, One-way ANOVA followed by Tukey's post hoc test for (FIG. 4D); Two-way ANOVA followed by Tukey's multiple-comparisons test for (FIG. 4G and FIG. 4H), Multiple t-tests for (FIG. 4I).

FIGS. 5A-5E. The generation of SEL1L C141Y mutation knock-in (KI) mouse. (FIG. 5A) Sequence alignment of the FNII domain across vertebrate species. Lines indicate conserved disulfide bond pairs. (FIG. 5B) Diagram of sgRNAs and homology-directed repair (HDR) donor used to generate SEL1L C141Y knock-in mice via CRISPR-Cas9 technology. Silent mutations were introduced to facilitate genotyping, and the “GGG” sequence was deleted in the HDR donor (indicated as “- - -”) to disrupt the PAM site. Forward primer for genotyping is indicated below the HDR donor sequence. (FIG. 5C) Sanger sequencing confirming the introduction of the SEL1L C141Y mutation along with surrounding silent mutations. (FIG. 5D) DNA agarose gel electrophoresis for genotyping of SEL1L C141Y knock-in mice. (FIG. 5E) Breeding strategy for SEL1L C141Y KI mice. Each established founder line was bred independently.

FIGS. 6A-6D. The alternative splicing of Sel1L occurs across different tissues in mice and is not affected by ER stress. (FIG. 6A to FIG. 6B) Diagram and DNA polyacrylamide gel electrophoresis (PAGE) analysis (FIG. 6A), and Quantification (FIG. 6B) of Sel1L exon 4 splicing in various mouse tissues. Statistics indicate the comparison between pancreas and other tissues. No statistically significant differences were observed between any of the other tissues. Hippo, Hippocampus. Cb, Cerebellum. BAT, Brown adipose tissue. n=3-4 mice/group. (FIG. 6C to FIG. 6D) DNA polyacrylamide gel electrophoresis (PAGE) analysis (FIG. 6C), and Quantification (FIG. 6D) of Sel1L exon 4 splicing in DMSO or Tunicamycin (Tuni)-treated mouse livers. n=3-4 mice/group. Data are represented as means±SEM. n.s., not significant. *p<0.05; **p<0.01; ***p<0.001, ****p<0.0001. One-way ANOVA followed by Tukey's post hoc test for (B), two-tailed t test for (FIG. 6D).

FIGS. 7A-7E. The Sel1L isoforms in KI and KI′ mice. (FIG. 7A) The diagram of the primer design for (FIG. 7B) and (FIG. 7D). (FIG. 7B to FIG. 7E) The agarose electrophoresis (FIG. 7B) and (FIG. 7D) and Quantitation (FIG. 7C) and (FIG. 7E) of Sel1L isoforms in WT, HET, and KI/KI′ mice. n=3-4 mice/each group. Data are represented as means±SEM. *p<0.05; **p<0.01; ***p<0.001, ****p<0.0001. One-way ANOVA followed by Tukey's post hoc test for (C and E).

FIGS. 8A-8F. SEL1L C141Y KI mice showed ERAD deficiency in the liver. (FIG. 8A) Western blot analysis of ERAD protein and ERAD substrates from WT and KI P0 pup liver from Line A and Line B. (FIG. 8B and FIG. 8C) Western blot analysis (FIG. 8B) and Quantitation (FIG. 8C) of ERAD protein and ERAD substrates from WT, HET, and KI P0 pup liver. n=3-4 mice/group. (FIG. 8D to FIG. 8E) Western blot analysis (FIG. 8D) and Quantitation (FIG. 8E) of SEL1L from WT, HET, and KI′ mouse liver. n=3-4 mice/group for KI line and 4-7 mice/group for KI′ line. Sex and age combined. (FIG. 8F) Diagram showing the epitope target regions of the home-made and Abcam anti-SEL1L antibodies on the SEL1L protein. Data are represented as means±SEM. n.s., not significant. *p<0.05; **p<0.01; ***p<0.001; ****p<0.0001. One-way ANOVA followed by Tukey's post hoc test for (FIG. 8C and FIG. 8E).

FIGS. 9A-9F. Confirmation of Sel1L exon 4 alternative splicing in the minigene reporter construct. (FIG. 9A) Sanger sequencing confirmation of the splice products shown in FIG. 2E and FIG. 2H. (FIG. 9B and FIG. 9F) DNA PAGE analysis of the WT and mutated minigene constructs as indicated by using primer pair F4B/R4. (FIG. 9C) Quantitation of Sel1L Exon 4a+4b transcript level in (FIG. 9B) and (FIG. 9F) normalized by total Sel1L transcript level as shown in FIG. 2E and FIG. 2H. (FIG. 9D and FIG. 9E) Quantitation of (FIG. 9B) and (FIG. 9F) as percent of exon 4 inclusion. n=3 independent biological replicates. Statistics indicate the comparison between WT and mutated constructs. *p<0.05; **p<0.01; ***p<0.001, One-way ANOVA followed by Tukey's post hoc test for (c), Two-way ANOVA followed by Tukey's multiple-comparisons test for (FIG. 9D to FIG. 9E).

FIGS. 10A-10G. Tissue weight and B cell profile in the spleen of SEL1L C141Y KI mice. (FIG. 10A to FIG. 10B) Tissue morphology (FIG. 10A) and tissue weights (FIG. 10B) of SEL1L C141Y knock-in (KI) pups at postnatal day 0 (P0). n=7-9 mice/group. (FIG. 10C) Morphology and weight quantitation of postprandial stomach in the P0 WT and KI pup. n=7-9 mice/group. (FIG. 10D) Body weight growth of female WT, HET, KI′ and surviving KI mice for postnatal week 1 to week 10. (FIG. 10E) Hematoxylin and eosin (H&E) staining of the spleen from SEL1L C141Y KI pups at P0. Yellow arrow, lymphocytes. (FIG. 10F) Quantitation of CD19+ signal area normalized by DAPI+area of the immunofluorescence staining in FIG. 3G. n=3 mice/group. (FIG. 10G) Percentage of neutrophils in the peripheral blood mononuclear cells (PBMCs) of WT, KI′ and surviving KI mice. Age and sex combined. n=4-11 mice/group. Data are represented as means±SEM. n.s., not significant. ****p<0.0001. One-way ANOVA followed by Tukey's post hoc test for (FIG. 10B and FIG. 10G), two-tailed t test for (FIG. 10C and FIG. 10F), Two-way ANOVA followed by Tukey's multiple-comparisons test for (FIG. 10D).

FIGS. 11A-11E. B cell deficiency in the circulation of SEL1L C141Y KI mice. (FIG. 11A) Gating strategy for flow cytometry. Cells were first gated for live cells using Zombie NIR viability dye, followed by singlet gating to exclude doublets. Live singlet cells were then gated for CD45⁺ leukocytes, and B cells were identified as CD19⁺B220⁺ within the CD45⁺ population. (FIG. 11B to FIG. 11D) Representative flow cytometry plots showing CD19⁺B220⁺ B cells in PBMCs from WT and KI pups at postnatal day 0 (P0) (FIG. 11B), and WT and KI′ pups at P0 (FIG. 11C) and 4 weeks of age (FIG. 11D). (FIG. 11E) Quantification of B cells as a percentage of CD45⁺ cells as shown in (FIG. 11D). n=3-4 mice/group. Data are represented as means±SEM. n.s., not significant. Two-tailed t test for (FIG. 11E).

FIGS. 12A-12D. SEL1L C141Y KI mice showed ERAD deficiency in the brain, which was reversed in KI′ mice. (FIG. 12A and FIG. 12B) Western blot analysis (FIG. 12A) and Quantitation (FIG. 12B) of ERAD protein and ERAD substrates from WT, HET, and KI P0 pup brain with quantitation on the right. n=3-4 mice/group. (FIG. 12C and FIG. 12D) Western blot analysis (FIG. 12C) and quantitation (FIG. 12D) of ERAD protein and ERAD substrates from the cortex of WT and KI′ mice with quantitation on the right. n=6-7 mice/group. Sex and age combined. Data are represented as means±SEM. n.s., not significant. *p<0.05; **p<0.01; ***p<0.001; ****p<0.0001. One-way ANOVA followed by Tukey's post hoc test for (FIG. 12B), Two-tailed t test for (FIG. 12D).

FIGS. 13A-13D. SEL1L C141Y KI mice did not cause overt UPR activation. (FIG. 13A) Western blot analysis and quantitation of ER chaperones BiP and PDI in P0 WT, HET and KI mouse brains. n=3-4 mice/group. (FIG. 13B) Phos-tag gel analysis of IRE1a phosphorylation in P0 WT, HET and KI mouse brains. n=3-4 mice/group. (FIG. 13C) Western blot analysis of PERK in P0 WT, HET and KI mouse brains. n=3-4 mice/group. (FIG. 13D) Electrophoresis analysis of XBP1 splicing of P0 WT, KI, and KI′ mouse brains with quantitation on the right. DNA PAGE gel for the XBP1 splicing and agarose gel for the internal control PPIA. n=3-6 mice/group. n=3-4 for positive controls. p, phosphorylated. 0, unphosphorylated. u, unspliced. s, spliced. Data are represented as means±SEM. n.s., not significant. ****p<0.0001. One-way ANOVA followed by Tukey's post hoc test for (FIG. 13A) and (FIG. 13D).

FIGS. 14A-14C. Sanger sequencing confirmation of ASO-mediated exon skipping in human patient fibroblast. Sanger sequencing confirmation of full-length band or skipping bands from patient fibroblast treated with control oligo (FIG. 14A), ASO1/3/4/5 (FIG. 14B), and ASO6/7 (FIG. 14C), as shown in FIG. 4C.

FIGS. 15A-15E. The regulation of SEL1L alternative splicing and exon skipping. (FIG. 15A) Quantification of transcript isoforms from the agarose gel analysis shown in FIG. 4E. n=5-6 independent biological replicates. (FIG. 15B) HEK293T cells were treated with control oligo or ASO1 at the indicated concentrations for 24 hours. SEL1L full-length and ΔExon 4 transcripts were analyzed by DNA agarose gel electrophoresis. (FIG. 15C) Quantitation of total SEL1L transcript level normalized by PPIA in FIG. 4E. n=4 independent replicates. (FIG. 15D and FIG. 15E) Western blot analysis (FIG. 15D) and Quantitation (FIG. 15E) of indicated proteins in WT, SEL1L C141Y, SEL1L C127Y and SEL1L FNII domain deletion (ΔFNII) HEK293T cells. n=3 technical replicates. Data are represented as means±SEM. n.s., not significant. *p<0.05; **p<0.01; ***p<0.001, ****p<0.0001. Two-way ANOVA followed by Tukey's multiple-comparisons test for (FIG. 15A and FIG. 15C), One-way ANOVA followed by Tukey's post hoc test for (FIG. 15E).

FIGS. 16A-16H. ASO-mediated exon skipping rescues ERAD defects in C141Y patient-derived fibroblasts. (FIG. 16A) A diagram showing the location of SEL1L C141Y mutation (red line) and Fibronectin II (FNII) domain on SEL1L gene. (FIG. 16B) The experimental design of the ASOs screen targeting SEL1L Exon 4. (FIG. 16C-FIG. 16D) DNA PAGE analysis of ASOs treatment at 10 μM for 3 days in C141Y patient fibroblasts, with a diagram showing corresponding products on the right and with quantification in (FIG. 16D). CON, Control ASO. Red shaded box and arrow indicates the location of SEL1L C141Y mutation. n=5 independent biological replicates. Statistics indicate the comparison between control ASO and ASO 1-7 targeting SEL1L exon 4 and introns 3 and 4. (FIG. 16E) Diagram showing the epitope target regions of the home-made (Ab2) and Abcam (Ab1) anti-SEL1L antibodies on the SEL1L protein in (FIG. 16F). (FIG. 16F-FIG. 16H) Western blot analysis (FIG. 16F) of ERAD proteins and ERAD substrates after ASO1 treatment at 10 μM for 3 days in human WT and C141Y patient fibroblasts, with quantification shown in (FIG. 16G-FIG. 16H). n=3-4 independent biological replicates. Data are represented as means±SEM. n.s., not significant. *p<0.05; **p<0.01; ***p<0.001, ****p<0.0001, by One-way ANOVA followed by Tukey's post hoc test for (D), Two-way ANOVA followed by Tukey's multiple-comparisons test for (FIG. 16G-FIG. 16H).

FIGS. 17A-17G. The alternative splicing of Sel1L exon 4 rescues ERAD dysfunction, perinatal lethality and B cell lymphopenia in SEL1L C141Y KI mice. (FIG. 17A) The diagram of the Sel1L isoforms and the primer design. (FIG. 17B-FIG. 17E) Western blot analysis of SEL1L (FIG. 17B), ERAD proteins and known ERAD substrate IRE1α (D FIG. 17) in WT, HET, KI, HET‘ and KI’ mouse livers with quantification in (FIG. 17C, SEL1L) and (FIG. 17E, ERAD proteins and substrate), respectively. n=4-20 mice/group. SEL1L from both antibodies were quantitated as average in (FIG. 17C). Asterisks, non-specific bands. (FIG. 17F) The survival rate of KI/KI′ pups and their WT and HET littermates at postnatal day 21 (P21). (FIG. 17G) Quantification of B cells as a percentage of CD45⁺ peripheral blood mononuclear cells (PBMCs) at P0 by flow cytometry. n=5-10 mice/group. Data are represented as means±SEM. n.s., not significant. *p<0.05; **p<0.01; ***p<0.001, ****p<0.0001, by One-way ANOVA followed by Tukey's post hoc test for (FIG. 17C, FIG. 17E, FIG. 17G), Chi-square test for (FIG. 17F).

FIGS. 18A-18E. The restoration of SEL1L or HRD1 could reduce ERAD dysfunction and partially rescue the development retardation and neurological defects observed in SEL1L S658P KI mice. (FIG. 18A) Western blot analysis of the hippocampus from WT mice injected with PBS (control) or AAV-PHP.eB-eGFP at dosage of 0.5, 1, 1.5, 2×10 vg for 4 weeks. (FIG. 18B) Immunofluorescence staining of WT mouse brain injected with 1×10¹² vg php.eb.AAV-eGFP for 4 weeks. Green, eGPF. Blue, DAPI. Arrow, cortex. Asterisk, Hippocampus. (FIG. 18C-FIG. 18D) Western blot analysis of the liver (FIG. 18C) and lung (FIG. 18D) of SEL1L and GFP from homozygous SEL1L S658P KI mice and WT littermate controls that were injected with single dose 1×10¹² vg php.eb.AAV-eGFP or php.eb.AAV-SEL1L for 5 weeks. (FIG. 18E) The growth curve of mouse body weight and clasping score of homozygous SEL1L S658P KI mice and WT littermate controls that were injected with single dose 1×10¹² vg php.eb.AAV-eGFP or php.eb.AAV-SEL1L and were tracked for 5 weeks FIG. 19. SEL1L-HRD1 ERAD is unregulated in AD neurons. Immunofluorescence staining of the cortex from WT 5×FAD mice, showing the upregulation of HRD1 expression in neurons with Aβ deposition. Blue, DAPI. Red, NeuN (neuron marker). Yellow, 6E10 antibody targeting Aβ. Green, HRD1.

FIGS. 20A-20B. Neuronal Sel1L deficiency altered amyloid pathologies in the 5×FAD model. (FIG. 20A) Tiled image of sagittal sections from 4-month-old 5×FAD (left) and Sel1LCamk2a; 5×FAD (right) mice. The amyloid is stained with a rabbit monoclonal antibody (Cell Signaling, D54D2). The section was counter-stained with hematoxylin (blue). (FIG. 20B) Representative images showing amyloid pathologies in the cortex, hippocampus, brain stem and cerebellum regions of mice at 4-month of age. Bar=100 μm. N=2 mice each.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, molecular biology, neuroscience, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, ±4%, ±3%, ±2%, etc.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to methods and compositions encompassed by the present disclosure refers to compositions and methods like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “isolated” means separated from constituents that otherwise may be present, for example, separated from bacterial stains or species that are not desired, or separating from other constituents that may be present with a micro-organism or cell in nature.

As used herein, the term “encode” refers to principle that DNA can be transcribed into RNA, which can then be translated into amino acid sequences that can form proteins

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient,” used interchangeably herein also refer to any mammalian subject for whom diagnosis, treatment, or therapy is desired, particularly humans. In embodiments, subject may relate to particular components of the subject, for instance specific tissues or fluids of a subject (e.g., human tissue in a particular area of the body of a living subject), which may be in a particular location of the subject, referred to herein as an “area of interest” or a “region of interest.”

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and includes cells in cell culture, cell supernatants, cell lysates, serum, plasma, biological fluids, and tissue samples.

As used throughout, the terms “nucleic acid,” “nucleic acid sequence,” “oligonucleotide,” “nucleotides,” “polynucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “gene” can refer to a hereditary unit corresponding to a sequence of DNA that occupies a specific location on a chromosome and that contains the genetic instruction for a characteristic(s) or trait(s) in an organism. The term “gene” can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, the term “recombinant” generally refers to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc.). Recombinant also refers to the polypeptide encoded by the recombinant nucleic acid. Non-naturally occurring nucleic acids or polypeptides include nucleic acids and polypeptides modified by man.

As used herein, “gene construct” or “construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc.), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The terms “transformation” and “transfection” mean the introduction of a nucleic acid, e.g., an expression vector, into a recipient cell including introduction of a nucleic acid to the chromosomal DNA of said cell, the nucleus of the cell, and/or the cytoplasm of the cell.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-87 (2013); Xie et al. “Adding amino acids to the genetic repertoire,” Curr. Opin. Chem. Biol. 9(6): 548-54 (2005); and all references cited therein. Beta and gamma amino acids are known in the art and are also contemplated herein as unnatural amino acids.

In accordance with standard nomenclature, amino acid residue sequences are denominated by either a three letter or a single letter code as indicated as follows, for example: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, 99%, or 100%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. In certain aspects, an SEL1L sequence can be the reference sequence, for example, NCBI Gene ID: NM_005065.6 (the reference sequence not having the following mutation: exon 4, c.422G>A, p.Cys141Tyr).

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-10 and Altschul et al. (1977) Nucleic Acids Res. 25: 3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)).

One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

As used herein, the term “biologically active fragments” or “bioactive fragment” of the polypeptides encompasses natural or synthetic portions of the full-length protein that are capable of specific binding to their natural ligand or of performing the function of the protein.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “vector” is a composition of matter which comprises a nucleic acid and which can be used to deliver the nucleic acid to the interior of a cell.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, poly amine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, an “essentially pure” preparation of a particular protein or peptide is a preparation wherein at least about 95%, and preferably at least about 99%, by weight, of the protein or peptide in the preparation is the particular protein or peptide.

A “subsequence”, “fragment” or “segment” is a portion of an amino acid sequence, comprising at least two or more amino acids, or a portion of a nucleic acid sequence comprising at least two or more nucleotide. The terms “subsequence”, “fragment” and “segment” are used interchangeably herein.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the 25 positions, e.g., 9 of 10, are matched or homologous, the two sequences share 90% homology. By way of example, the DNA sequences 3′ ATTGCC5′ and 3′TATGGC share 50% homology.

As used herein, “homology” is used synonymously with “identity.”

An “isolated polypeptide” refers to a polypeptide, or segment or fragment thereof, which has been separated from a naturally occurring state and/or that is present in a substantially purified form. In some embodiments, an isolated polypeptide refers to a polypeptide that has been isolated from one or more substances otherwise present in an artificial reaction by which the polypeptide is produced or employed (e.g., an in vitro expression reaction).

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell and/or which might be otherwise present in an artificial reaction by which the nucleic acids are produced or employed. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light scattering.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

A “disease” is a state of health of an animal wherein the animal (i.e., a mammal such as a human) cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. A “condition” encompasses both diseases and disorders.

The terms “co-administration” or “co-administered” as used herein refer to the administration of a composition described herein along with any one or more reagents that are utilized in diagnosis and/or treatment. In some embodiments, the co-administration is concurrent. In other embodiments, a composition or reagent is administered prior to a second composition or reagent in this aspect, each component may be administered separately, but sufficiently close in time to provide the desired effect. Those of skill in the art understand that the formulations of the various enzymes whose use is described herein may vary. The appropriate dosage for co-administration can be readily determined by one skilled in the art.

The term “composition” as used herein refers to a product comprising the specified ingredients (i.e., one or more enzymes described herein) in the specified amounts, as well as any product which results, directly or indirectly, from combination of the specified ingredients in the specified amounts. Such a term in relation to a composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Those skilled in the art will appreciate that the term “composition”, as used herein, can be used to refer to a discrete physical entity that comprises one or more specified components. In general, unless otherwise specified, a composition can be of any suitable form—e.g., gel, liquid, solid, etc.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

A composition of the disclosure can be a liquid solution, suspension, emulsion or a powder. Compositions described herein may include sterile aqueous or non-aqueous solvents, such as water, isotonic saline, isotonic glucose solution, buffer solution, or other solvents conveniently used for parenteral administration of therapeutically active agents, stabilizers, buffers, or preservatives, e.g. antioxidants such as methylhydroxybenzoate or similar additives.

A composition of the disclosure may be sterilized by, for example, addition of sterilizing agents to the composition, irradiation of the composition, or heating the composition.

Alternatively, the compounds or compositions of the present disclosure may be provided as sterile solid preparations e.g. lyophilized powder, which are readily dissolved in sterile solvent immediately prior to use.

The term “freeze-dried (lyophilized) as used herein refers to a preparation of a component described herein, for example, nucleic acids or enzymes, that have been initially frozen and the water content removed by vacuum.

The term “reducing” means to diminish in extent, amount, or degree.

As used herein, an “isolated nucleic acid molecule”, “isolated polynucleotide”, and “isolated nucleic acid fragment” may be used interchangeably and refer to a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

As used herein, the term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes. As used herein, “synthetic genes” can be assembled from oligonucleotide building blocks that are chemically synthesized using procedures known to those skilled in the art. These building blocks are ligated and annealed to form gene segments that are then enzymatically assembled to construct the entire gene.

As used herein, the term “chemically synthesized”, as pertaining to a DNA sequence, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequences to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

The term “variant” refers to an amino acid or peptide sequence having conservative amino acid substitutions, non-conservative amino acid substitutions (i.e., a degenerate variant), substitutions within the wobble position of each codon (i.e., DNA and RNA) encoding an amino acid, amino acids added to the C-terminus of a peptide, or a peptide having 60%, 70%, 80%, 90%, 95%, 96%, 97%, 98%, 99%, or 100% sequence identity to a reference sequence.

In some embodiments, suitable ASOs targeting SEL1L or sgRNAs for CRISPR methods may include sequences comprising a nucleotide sequence having at least 80%, 85%, 90%, 91%, 92%, 93%, 94%, 95%, 96%, 97%, 98%, or 99% sequence identity to any of the nucleotide sequences reported herein.

In some embodiments, suitable isolated nucleic acid molecules encode a protein having an amino acid sequence that is at least about 20%, preferably at least 30%, 33%, 40%, 50%, 60%, 70%, 80%, 85%, 90%, 91%, 92%, 93%, 94%, 95%, 96%, 97%, 98%, or 99% identical to the amino acid sequences reported herein. Suitable nucleic acid molecules not only have the above homologies, but also typically encode a protein having about 210 to 380 amino acids in length, about 300 to about 360 amino acids, preferably about 310 to about 350 amino acids, and most preferably about 320 to about 335 amino acids in length.

The terms “isolated”, “purified”, or “biologically pure” as used herein, refer to material that is substantially or essentially free from components that normally accompany the referenced material in its native state (in particular free from its naturally-occurring state).

The term “clinical well-being” as used herein, refers to a state or degree of clinical or physiological wellness or health of a patient. A clinician can evaluate a patient's clinical well-being by physical examination or performing one or more tests or assays.

“Inhibitors,” “activators,” and “modulators” of expression or of activity are used to refer to inhibitory, activating, or modulating molecules, respectively, identified using in vitro and in vivo assays for expression or activity of a described target protein (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or more higher.

The terms “administering,” “delivering,” and “introducing,” can be used interchangeably to indicate the introduction of a diagnostic or therapeutic composition or agent (e.g., compositions comprising one or more agent described herein) into the body of a subject. The therapeutic composition or agent can be administered through any appropriate means that results in the delivery of at least a portion of the composition or agent to a desired location in the subject such that the composition or agent retains its therapeutic capability. Useful methods of delivering the therapeutic include, but are not limited to, intravenous delivery, subcutaneous delivery, intradermal delivery, intracoronary delivery, intracardiac delivery, oral delivery, or any combination thereof.

The term “administered continuously” refers to the continuous delivery of a therapeutic agent, e.g., compound, molecule, peptide, biologic, chemical, etc. over a 24-hour period or more.

The term “therapeutically effective amount” refers to an amount of therapeutic agent effective to treat at least one symptom of a disease or disorder in a subject. In other words, such an amount is sufficient to bring about a beneficial or desired clinical effect. The “therapeutically effective amount” of the agent for administration may vary based upon the desired activity, the diseased state of the subject being treated, the dosage form, method of administration, subject factors such as the subject's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

As used herein, the terms “pharmaceutically acceptable” or “pharmacologically acceptable” refer to compositions that do not substantially produce adverse reactions, e.g., toxic, allergic, or immunological reactions, when administered to a subject.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used in the specification and claims includes one and more such excipients, diluents, carriers, and adjuvants.

As used herein, a “pharmaceutical composition” is meant to encompass a composition or pharmaceutical composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

The terms “therapy,” “treatment,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

As used herein, a “chemically modified” amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

A “spacer” as used herein refers to a peptide that joins the proteins comprising a fusion protein. Generally, a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polynucleotide or polypeptide (including antibodies) or receptor, refers to a binding reaction which is determinative of the presence of the protein or polypeptide or receptor in a heterogeneous population of proteins and other biologics. Thus, under designated conditions (e.g., immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule. As used herein “target” means to design a polynucleotide or polypeptide so that it specifically binds to its target.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically, specific delivery results in a much stronger association between the delivered molecule and cells bearing the target molecule than between the delivered molecule and cells lacking the target molecule.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “protein domain” refers to a portion of a protein, portions of a protein, or an entire protein showing structural integrity; this determination may be based on amino acid composition of a portion of a protein, portions of a protein, or the entire protein.

The term “gapmer” refers short DNA antisense oligonucleotide structures with RNA-like segments on both sides of the sequence. In certain aspects, gapmers can comprise a short DNA strand flanked by strands of RNA mimics. The mimics can comprise locked nucleic acids (LNA), 2′-Ome (MOE), or 2′-F modified bases. LNA sequences are RNA analogues that can be “locked” into an ideal Watson-Crick base pairing conformation. In certain aspects, gapmers can comprise nucleotides modified with phosphorothioate (PS) groups

### Discussion

The SEL1L-HRD1 endoplasmic reticulum-associated degradation (ERAD) pathway is essential for maintaining protein homeostasis. Genetic variants and mutations that impair SEL1L-HRD1 function cause severe congenital disorders, including ERAD-associated neurodevelopmental disorder (ENDI) and ENDI-Agammaglobulinemia (ENDI-A). ENDI patients presented invariably intellectual disability, developmental delay, short stature, underweight, facial dysmorphisms and locomotor dysfunctions. A specific severe form of ENDI, ENDI-A patients presented additional symptoms, including frequent infections, agammaglobulinemia, B cell lymphopenia, and early death. Consistently, mouse carrying biallelic pathogenic SEL1L mutations (SEL1L S658P mutation and C141Y mutation) also exhibited similar phenotypes, including perinatal lethality, developmental delay, ataxia, and immune deficiency. These data further showed that restoration of SEL1L-HRD1 activity, either via ASO-mediated exon skipping or AAV-mediated overexpression, is a viable therapeutic strategy.

Conversely, aberrant or maladaptive activation of the SEL1L-HRD1 pathway may contribute to neurodegenerative disease. Preliminary data showed that in an Alzheimer's disease (AD) mouse model, SEL1L-HRD1 defects may be associated with reduced amyloid p plaque accumulation, suggesting SEL1 L-HRD1 ERAD to be a potential therapeutic target in AD.

Together, these findings demonstrate that precise modulation of SEL1L-HRD1 ERAD offers therapeutic benefit across both genetic ENDI disorders and neurodegenerative diseases.

Described herein is therapeutic modulation of the SEL1L-HRD1 ERAD pathway based on demonstrated correction of disease phenotypes in mouse models and patient-derived cells, for example. Antisense oligonucleotide (ASO)-induced skipping of SEL1L exon 4 rescues ERAD deficiency and multiple physiological defects in ENDI-A mice and restores ERAD function in SEL1L C141Y patient fibroblasts. AAV-mediated SEL1L overexpression ameliorates neurological and systemic abnormalities in SEL1L S658P knock-in mice. Conversely, neuronal specific deletion of SEL1L reduces Alzheimer's disease pathology in 5×FAD mice. These findings support therapeutic applications of SEL1L-HRD1 modulation across ERAD-deficient disorders and neurodegenerative disease.

### I. THERAPEUTICS

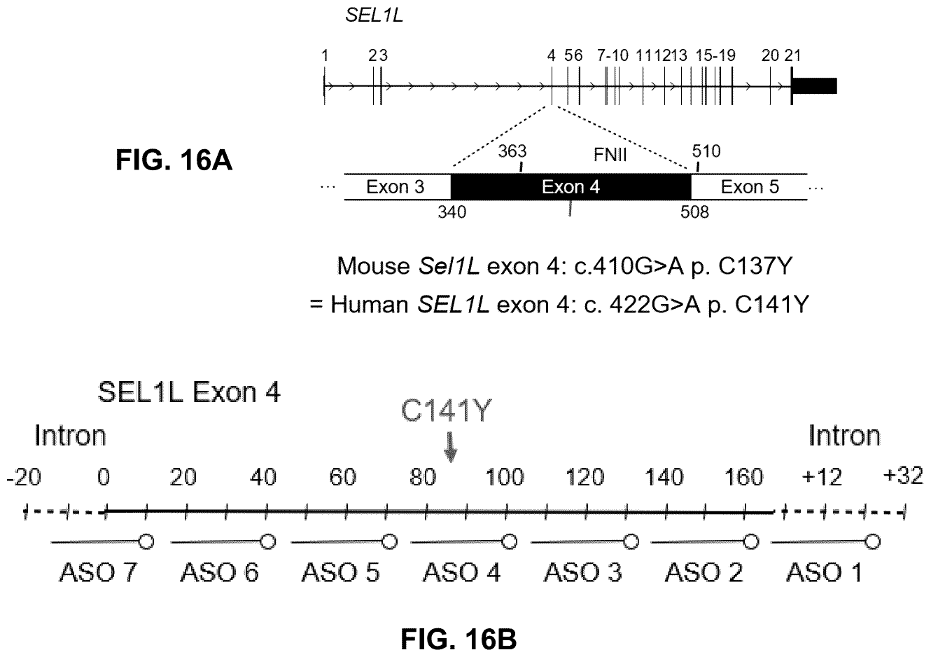

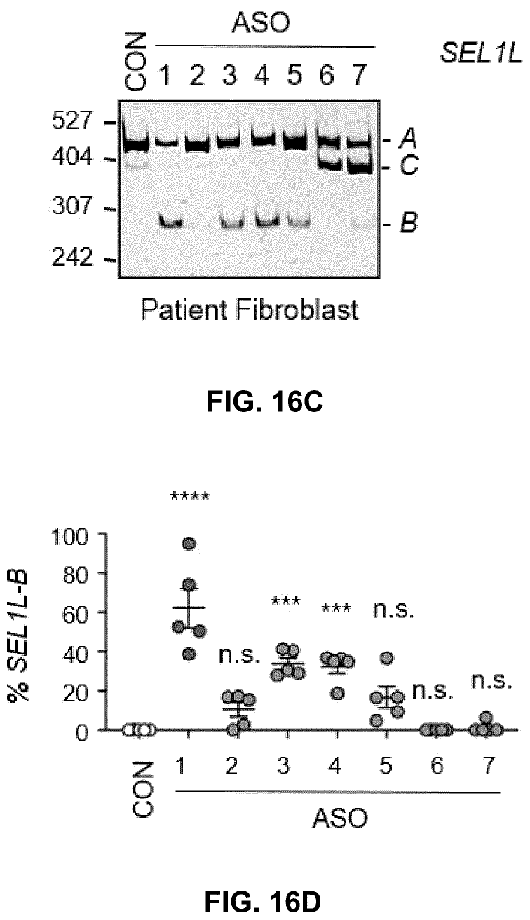

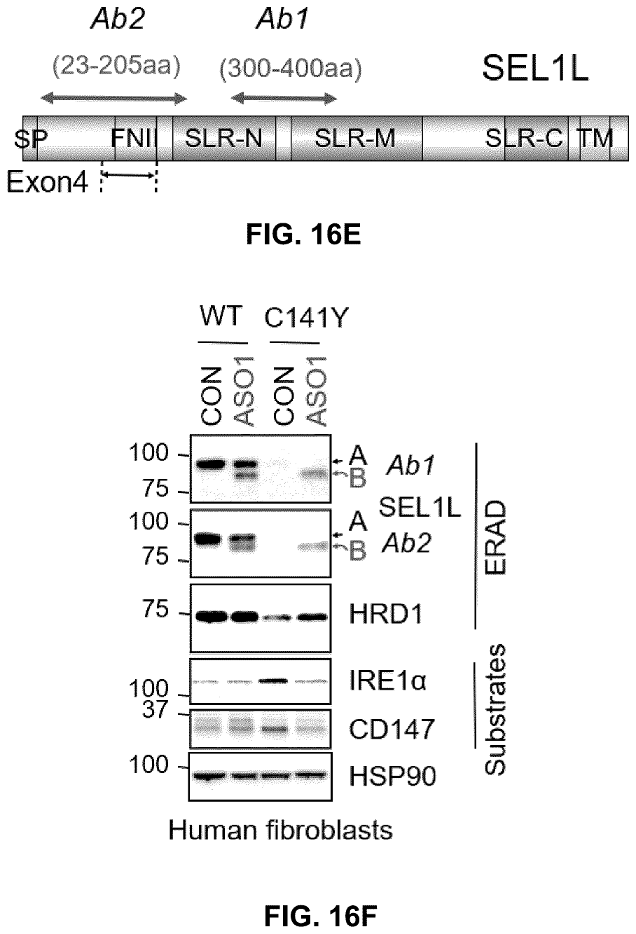

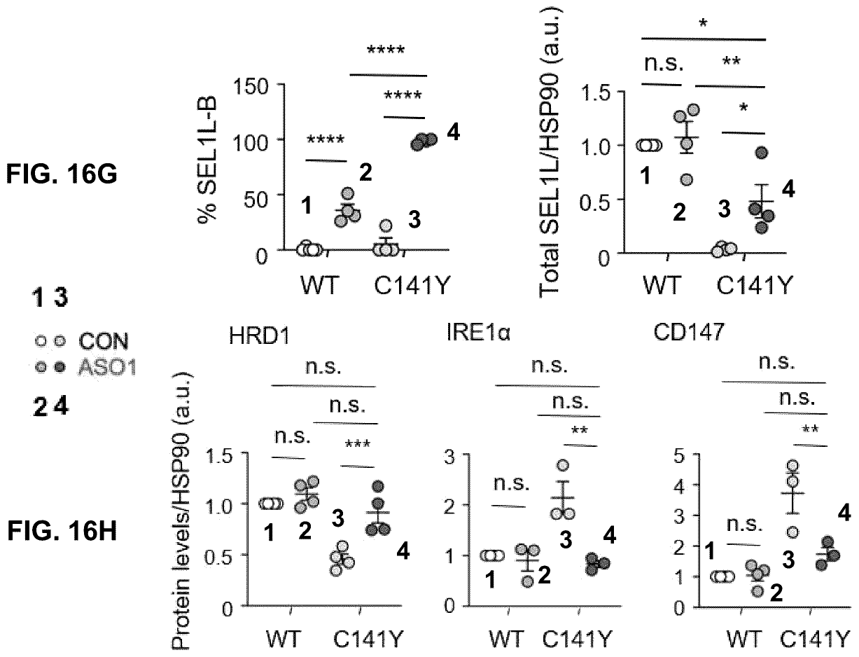

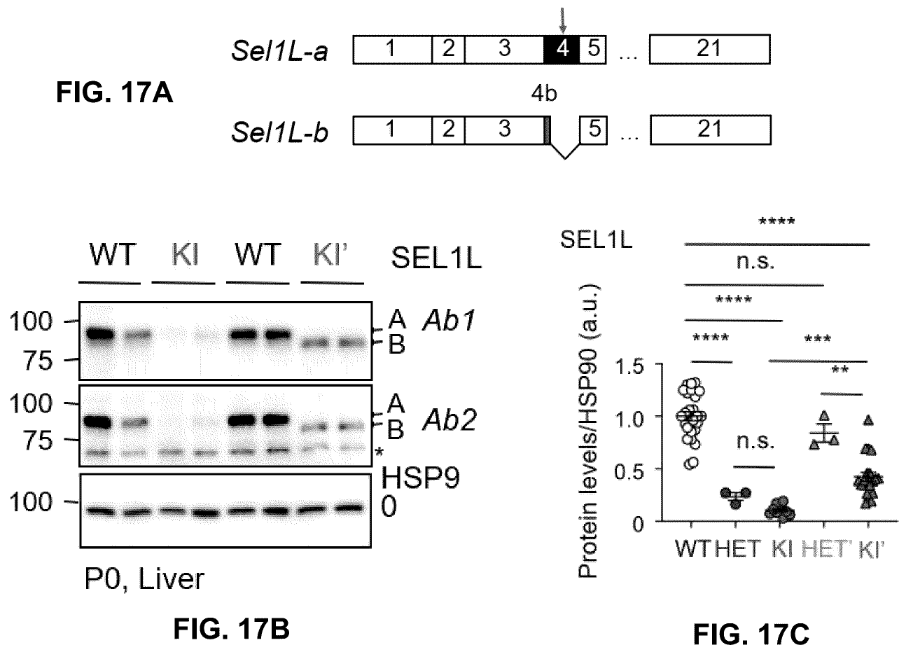

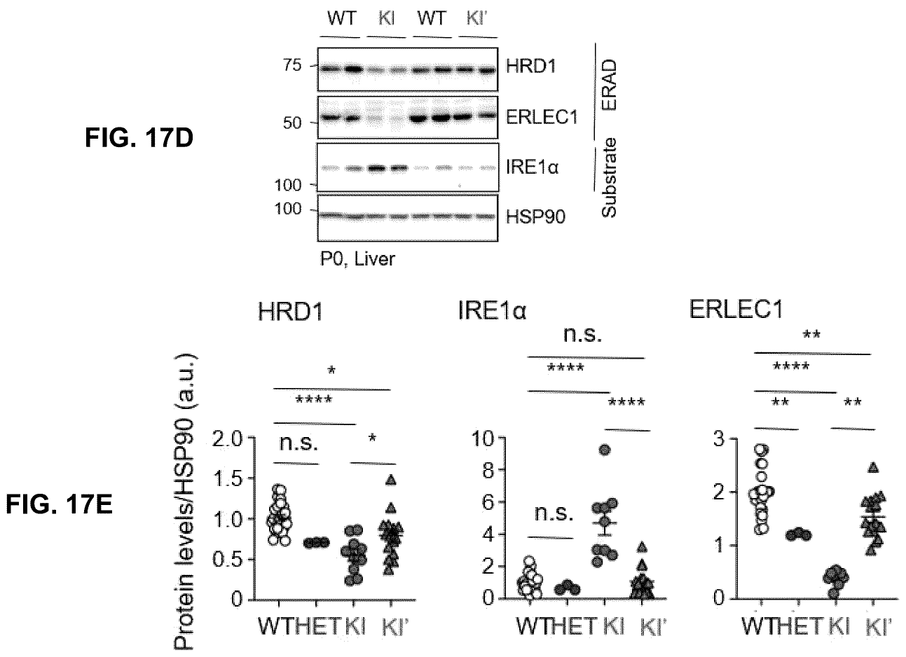

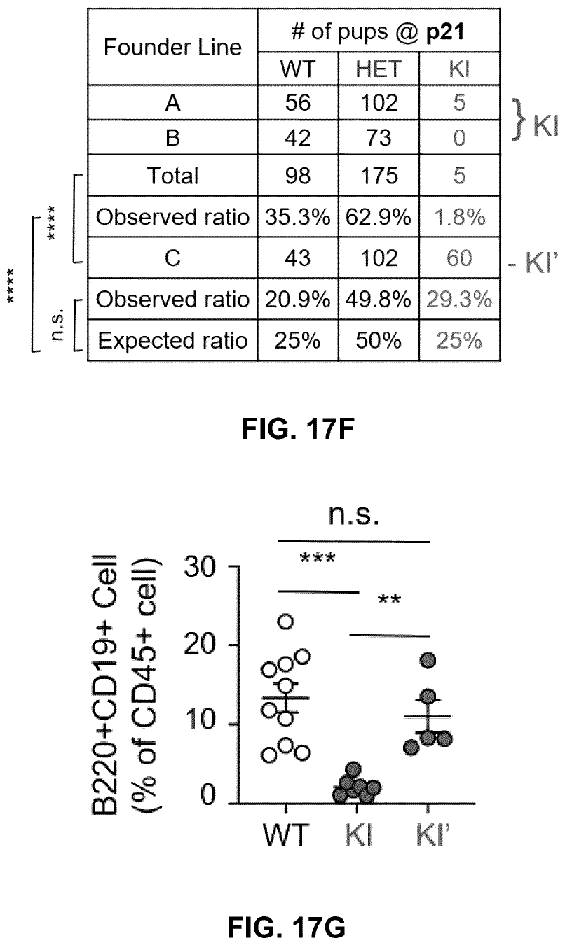

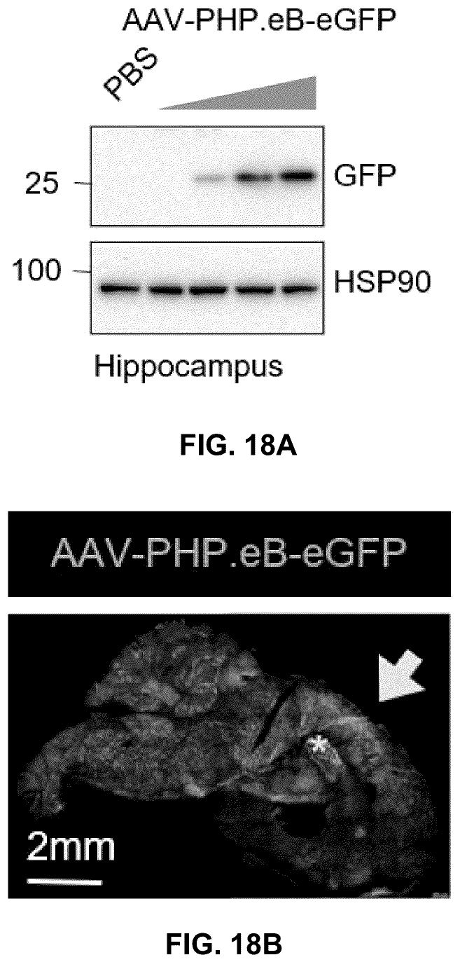

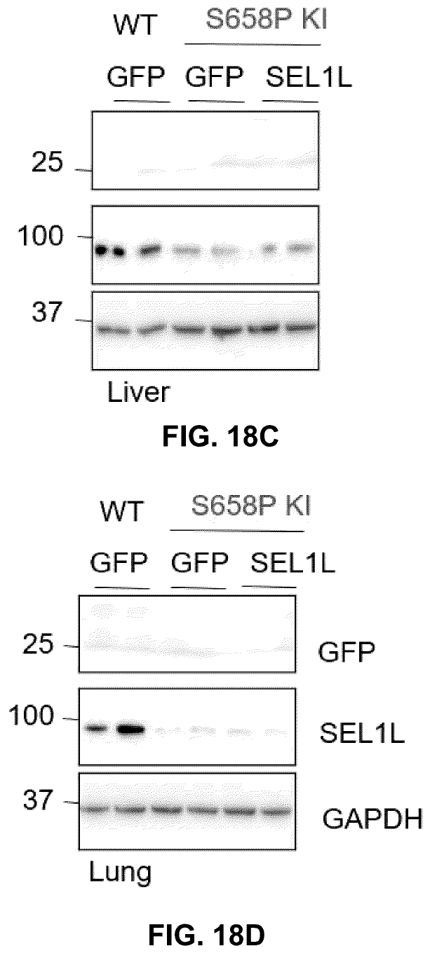

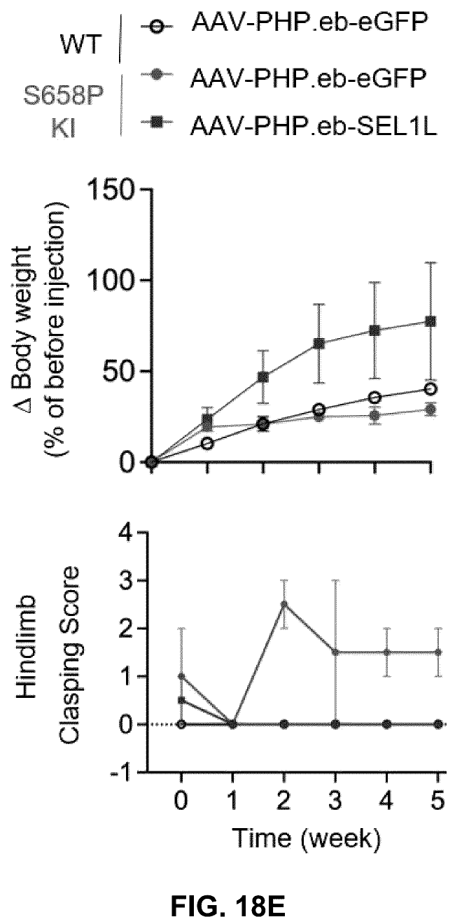

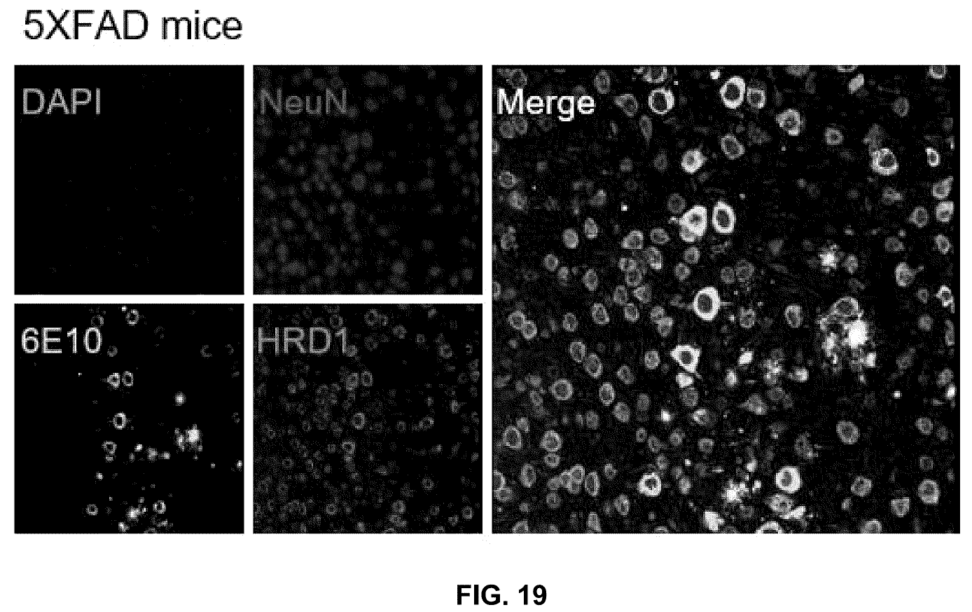

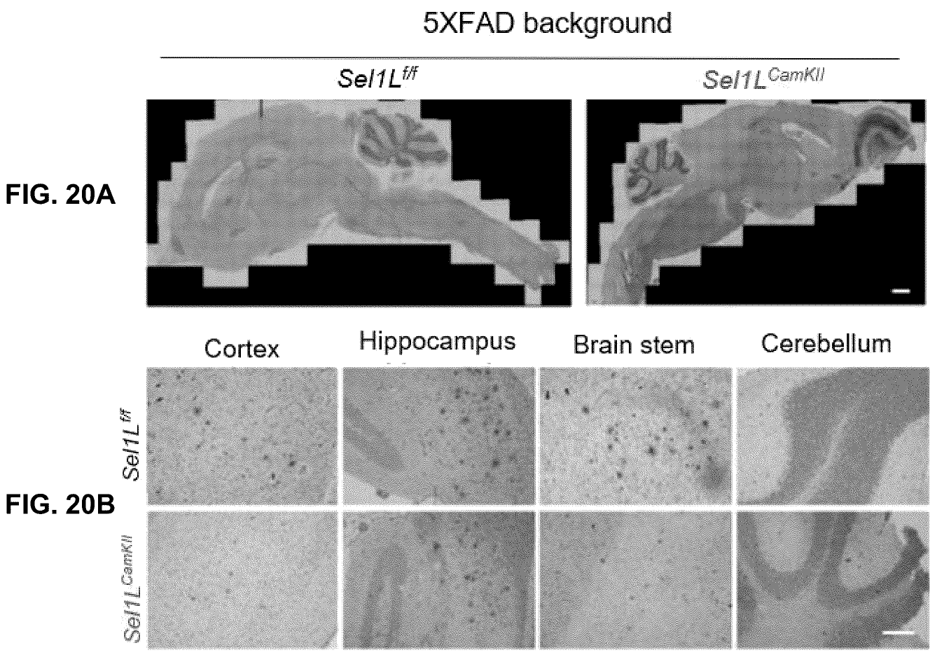

The present disclosure provides therapeutic compositions and methods for modulating SEL1L-HRD1 ERAD activity based on demonstrated rescue of disease phenotypes in animal models and patient cells. At least the following therapeutic strategies are described herein: A. Splice-Modulating ASOs for SEL1L Exon 4 Skipping (FIGS. 16A-16H, FIGS. 17A-17F); B. AAV-Mediated Gene Therapy for SEL1L Overexpression (FIGS. 18A-18E); and C. Neuron Specific (i.e., Neuronal) SEL1L Inhibition for Alzheimer's Disease (FIG. 19).

### A. Splice-Modulating Antisense-Oligonucleotides (ASOs)

Antisense oligonucleotides (ASO) that induces] SEL1L exon 4 skipping restore ERAD activity and correct phenotypes in patient fibroblasts carrying the SEL1L C141Y mutation. Genetic modulation of Sel1L exon 4 alternative splicing (mimic ASO-mediated exon 4 skipping in patient fibroblast) in ENDI-A mice (homozygous Sel1L C141Y knock-in mice) rescues perinatal lethality, B-cell lymphopenia, and ERAD deficiency. ASOs designed to induce exon 4 skipping of SEL1L pre-mRNA. Such ASO's are shown to restore ERAD function in ENDI-A mice and C141Y patient fibroblasts. Such ASOs are useful for treating SEL1L-exon-4-related ERAD-deficiency disorders, for example. Also described herein are gapmer ASOs for the same purpose.

Described in the present example is ASO Exon-Skipping Therapy. In an embodiment, described in the present example is a method of treating a SEL1L-associated ERAD-deficiency disorder in a subject, comprising administering an antisense oligonucleotide that induces skipping of SEL1L exon 4 (or any other exons), thereby restoring (or regulating) ERAD function. In certain aspects, a SEL1L-associated ERAD-deficiency disorder comprises a SEL1L mutation. In certain aspects, the SEL1L mutation comprises an ENDI-A exon 4 mutation. In certain aspects, the SEL1L mutation comprises C141Y. In certain aspects, the treatment rescues one or more phenotypes selected from: ERAD deficiency, perinatal lethality, or B-cell lymphopenia.

In embodiments, ASOs according to the present disclosure can comprise, consist essentially of, or consist of one or more nucleotides having at least 80-100%, 85-100%, 90-100%, 91-100%, 92-100%, 93-100%, 94-100%, 95-100%, 96-100%, 97-100%, 98-100%, 99-100%, 80%, 85%, 86%, 87%, 88%, 89%, 90%, 91%, 92%, 93%, 94%, 95%, 96%, 97%, 98%, 99%, or 100% sequence identity to one or more of SEQ ID NOs: 1-7.

Also described herein are vectors comprising ASOs as described herein that can be utilized to deliver an ASO into a target cell. Such vectors include, without intending to be limiting, lentiviral vectors, plasmids, adeno-associated viral (AAV) vectors, and the like.

In embodiments, gapmer ASOs according to the present disclosure can comprise, consist essentially of, or consist of one or more nucleotides having at least 80-100%, 85-100%, 90-100%, 91-100%, 92-100%, 93-100%, 94-100%, 95-100%, 96-100%, 97-100%, 98-100%, 99-100%, 80%, 85%, 86%, 87%, 88%, 89%, 90%, 91%, 92%, 93%, 94%, 95%, 96%, 97%, 98%, 99%, or 100% sequence identity to one or more of SEQ ID NOs: 8-19. In certain aspects, any one or more of the first one to five nucleotides on the 5′ end of the gapmer ASOs, and one or more of the terminal one to five nucleotides on the 3′ end can be modified. In certain aspects, the modification comprises a locked nucleic acids (LNA), 2′-Ome (MOE), or 2′-F modification. In embodiments, the modification is a MOE modification on each of the first five nucleotides on the 5′ end and terminal five nucleotides on the 3′ end.

### B. AAV-Mediated Gene Therapy

AAV-PHP.eB-mediated SEL1L overexpression via i.v. injection, for example and without intending to be limiting, can rescue growth retardation and hindlimb clasping in SEL1L S658P knock-in mice. AAV vectors encoding SEL1L for delivery to the CNS. Shown to rescue body-weight and motor deficits in SEL1L S658P mutant mice. Other therapeutic administration routes are also contemplated by the present disclosure, or example, intracerebroventricular or intraparenchymal administration.

Described in the present example is AAV SEL1L Overexpression Therapy. In an embodiment, described herein is a method of treating a SEL1L loss-of-function mutation in a subject, comprising administering an adeno-associated virus (AAV) vector encoding SEL1L to increase SEL1L expression in the central nervous system and restore ERAD function. In certain aspects, the SEL1L mutation is S658P. In certain aspects, the AAV vector is administered intracerebroventricularly or intraparenchymally. In certain aspects, the treatment can improve body weight or neurological symptoms.

In embodiments, SEL1L overexpression can be accomplished by expressing a SEL1L construct (i.e., a full-length SEL1L protein or active fragment thereof expressed from a coding sequence encoding such) using a vector. In embodiments, an SEL1L construct according to the present disclosure can comprise, consist essentially of, or consist of one or more nucleotides having at least 80-100%, 85-100%, 90-100%, 91-100%, 92-100%, 93-100%, 94-100%, 95-100%, 96-100%, 97-100%, 98-100%, 99-100%, 80%, 85%, 86%, 87%, 88%, 89%, 90%, 91%, 92%, 93%, 94%, 95%, 96%, 97%, 98%, 99%, or 100% sequence identity to one or more of SEQ ID NOs: 20-21.

The SEL1L constructs according to the present disclosure can be cloned, for example, into an expression vector, such as a plasmid, lentiviral vector, or adeno-associated viral (AAV) vector for expression in a host cell.

In certain aspects, AAV-PHP.eB vectors that can be utilized (i.e. into which SEL1L sequences and active fragments thereof can be cloned into) include those available from a number of sources, for example, Addgene (Catalog #37825-PHPeB; with pUCmini-iCAP-PHP.eB (plasmid #103005)) and Creative Biogene (AAV PHP.eB-CAG-GFP, catalog AAV00305Z).

Additionally aspects of utilization of AAV-PHP.eB vectors can be found, for example, in the literature, for example, in: Chan K Y, Jang M J, Yoo B B, Greenbaum A, Ravi N, Wu W L, Senchez-Guardado L, Lois C, Mazmanian S K, Deverman B E, Gradinaru V. Engineered AAVs for efficient noninvasive gene delivery to the central and peripheral nervous systems. Nat Neurosci. 2017 August; 20(8):1172-1179. doi: 10.1038/nn.4593. Epub 2017 Jun. 26. PMID: 28671695; PMCID: PMC5529245 and Arpad Palfi, Naomi Chadderton, Sophia Millington-Ward, Iris Post, Pete Humphries, Paul F. Kenna, G. Jane Farrar, AAV-PHP.eB transduces both the inner and outer retina with high efficacy in mice, Molecular Therapy—Methods & Clinical Development, Volume 25, 2022, Pages 236-249, ISSN 2329-0501, https://doi.org/10.1016/j.omtm.2022.03.016; Chatterjee, D., Marmion, D. J., McBride, J. L. et al. Enhanced CNS transduction from AAV.PHP.eB infusion into the cisterna magna of older adult rats compared to AAV9. Gene Ther 29, 390-397 (2022). https://doi.org/10.1038/s41434-021-00244-y; the contents of both of which relating to AAV-PHP.eB vectors are incorporated by reference as if fully set forth herein.

### C. Neuron-Specific SEL1L Suppression

Selective SEL1L reduction in excitatory neurons can attenuate AD pathology in 5×FAD mice. Therapeutic agents may include RNAi, gapmer antisense oligonucleotides, or CRISPR-based inhibitors.

Described in the present example is SEL1L Inhibition for Alzheimer's Disease. In an embodiment, described herein is a method of treating Alzheimer's disease in a subject, comprising reducing SEL1L expression or activity in excitatory neurons, thereby decreasing Alzheimer's disease pathogenesis. In certain aspects, SEL1L expression or activity is reduced using RNA interference. In certain aspects, SEL1L expression or activity is reduced using an antisense oligonucleotide.

### D. SEL1L Suppression Via Gene Editing

In some embodiments, SEL1L exon skipping or suppression can be undertaken using one or more gene editing techniques, for example, such as those described below.

Many genome modifying systems and corresponding nucleases may be used, for example, without limitations, a homing nuclease polypeptide; a FokI polypeptide; a transcription activator-like effector nuclease (TALEN) polypeptide; a MegaTAL polypeptide; a meganuclease polypeptide; a zinc finger nuclease (ZFN); an ARCUS nuclease; and the like. The meganuclease can be engineered from an LADLIDADG homing endonuclease (LHE). A megaTAL polypeptide can comprise a TALE DNA binding domain and an engineered meganuclease. See, e.g., International Patent Application Publication No. WO 2004/067736 (homing endonuclease); Urnov et al. (2005) Nature 435:646 (ZFN); Mussolino et al. (2011) Nucle. Acids Res. 39:9283 (TALE nuclease); Boissel et al. (2013) Nucl. Acids Res. 42:2591 (MegaTAL). In some embodiments, the genome modifying system is a CRISPR-Cas system comprising a CRISPR-Cas nuclease and guide polynucleotides, e.g., guide RNAs (gRNAs). In some embodiments, for example, the CRISPR-Cas nuclease is CRISPR-Cas9. See, e.g., Møller, Henrik Devitt et al. Nucleic Acids Research (2018).

The CRISPR-Cas nuclease can be any of a variety of CRISPR-Cas nucleases. CRISPR-Cas nucleases can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida. Suitable CRISPR-Cas nucleases are described in detail below.

Examples of CRISPR-Cas nucleases are CRISPR-Cas endonucleases (e.g., class 2 CRISPR-Cas nucleases such as a type II, type V, or type VI CRISPR-Cas nuclease). In some embodiments, the CRISPR-Cas nuclease is a type II CRISPR-Cas nuclease. In some embodiments, the type II CRISPR-Cas nuclease is a Cas9 polypeptide. In some embodiments, the CRISPR-Cas nuclease is a type V CRISPR-Cas nuclease, e.g., a Cas12a, a Cas12b, a Cas12c, a Cas12d, a Cas12e, a Cpf1, a C2c1, or a C2c3 polypeptide. In some embodiments, the CRISPR-Cas nuclease is a type VI CRISPR-Cas nuclease, e.g., a Cas13a, a Cas13b, a Cas13c, a Cas13d, a C2c2 (also referred to as Cas13a) polypeptide. In some embodiments, the CRISPR-Cas nuclease is a Cas14 polypeptide. In some embodiments, the CRISPR-Cas nuclease is a Cas14a polypeptide, a Cas14b polypeptide, or a Cas14c polypeptide. In some embodiments, a suitable CRISPR-Cas nuclease is a CasX or a CasY polypeptide. CasX and CasY polypeptides are described in Burstein et al. (2017) Nature 542:237.

Also suitable for use is a variant CRISPR-Cas nuclease, where the variant is a high-fidelity or enhanced specificity CRISPR-Cas nuclease with reduced off-target effects and robust on-target cleavage. Non-limiting examples of CRISPR-Cas nuclease variants with improved on-target specificity include the SpCas9 (K855A), SpCas9 (K810A/K1003A/R1060A) (also referred to as eSpCas9(1.0)), and SpCas9 (K848A/K1003A/R1060A) (also referred to as eSpCas9(1.1)) variants described in Slaymaker et al. Science, 351(6268):84-8 (2016), and the SpCas9 variants described in Kleinstiver et al. Nature, 529(7587):490-5 (2016) containing one, two, three, or four of the following mutations: N497A, R661A, Q695A, and Q926A (e.g., SpCas9-HF1 contains all four mutations).

Also suitable for use, e.g., when fused with a second enzyme with nicking of DNA cleaving activity, is a variant CRISPR-Cas nuclease, where the variant CRISPR-Cas nuclease has reduced or no nucleic acid cleavage activity. For example, the dCas9 variant (Jinek et al. Science, 2012, 337:816-821; Qi et al. Cell, 152(5):1173-1183) contains two silencing mutations of the RuvC1 and HNH nuclease domains (D10A and H840A). In some embodiments, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Application Publication No. WO 2013/176772. The dCas9 enzyme can contain a mutation at D10, E762, H983, or D986, as well as a mutation at H840 or N863. In some instances, the dCas9 enzyme can contain a D10A or D10N mutation. Also, the dCas9 enzyme can contain a H840A, H840Y, or H840N. In some embodiments, the dCas9 enzyme can contain D10A and H840A; D10A and H840Y; D10A and H840N; D10N and H840A; D10N and H840Y; or D10N and H840N substitutions. The substitutions can be conservative or non-conservative substitutions to render the Cas9 polypeptide catalytically inactive and able to bind to target nucleic acid, thereby inhibiting activity of the target nucleic acid (a TREM1 or PGE2-EP2 receptor, or both, for example).

In many embodiments, guide polynucleotides, e.g., gRNAs, are used with a CRISPR-Cas nuclease. In some embodiments, a guide polynucleotide, e.g., gRNA, comprises a sequence having at least 70%, at least 80%, at least 85%, at least 90%, at least 95%, or 100% identity to a sequence at a target site. In some embodiments, the target site is on the genomic DNA of a host cell. In some embodiments, a gRNA library is used with the CRISPR-Cas nuclease. A non-limiting example of a gRNA library is the CRISPR-Cas9 MinLib plasmid library (MinLibCas9 Library, Addgene #164896).

As used throughout, the term “Cas9 polypeptide” means a Cas9 protein or a fragment thereof present in any bacterial species that encodes a Type II CRISPR/Cas9 system. See, for example, Makarova et al. Nature Reviews, Microbiology, 9: 467-477 (2011), including supplemental information, hereby incorporated by reference in its entirety. For example, the Cas9 protein or a fragment thereof can be from Streptococcus pyogenes. Full-length Cas9 is an endonuclease comprising a recognition domain and two nuclease domains (HNH and RuvC, respectively) that creates double-stranded breaks in DNA sequences. In the amino acid sequence of Cas9, HNH is linearly continuous, whereas RuvC is separated into three regions, one left of the recognition domain, and the other two right of the recognition domain flanking the HNH domain. Cas9 from Streptococcus pyogenes is targeted to a genomic site in a cell by interacting with a guide RNA that hybridizes to a 20-nucleotide DNA sequence that immediately precedes an NGG motif recognized by Cas9. This results in a double-strand break in the genomic DNA of the cell.

As used throughout, a dCas9 polypeptide is a deactivated or nuclease-dead Cas9 (dCas9) that has been modified to inactivate Cas9 nuclease activity. Modifications include, but are not limited to, altering one or more amino acids to inactivate the nuclease activity or the nuclease domain. For example, and not to be limiting, D10A and H840A mutations can be made in Cas9 from Streptococcus pyogenes to inactivate Cas9 nuclease activity. Other modifications include removing all or a portion of the nuclease domain of Cas9, such that the sequences exhibiting nuclease activity are absent from Cas9. Accordingly, a dCas9 may include polypeptide sequences modified to inactivate nuclease activity or removal of a polypeptide sequence or sequences to inactivate nuclease activity. The dCas9 retains the ability to bind to DNA even though the nuclease activity has been inactivated. Accordingly, dCas9 includes the polypeptide sequence or sequences required for DNA binding but includes modified nuclease sequences or lacks nuclease sequences responsible for nuclease activity. It is understood that similar modifications can be made to inactivate nuclease activity in other site-directed nucleases, for example in Cpf1 or C2c2.

In some examples, the dCas9 protein is a full-length Cas9 sequence from S. pyogenes lacking the polypeptide sequence of the RuvC nuclease domain and/or the HNH nuclease domain and retaining the DNA binding function. In other examples, the dCas9 protein sequences have at least 30%, 40%, 50%, 60%, 70%, 80%, 90%, 95%, 98% or 99% identity to Cas9 polypeptide sequences lacking the RuvC nuclease domain and/or the HNH nuclease domain and retains DNA binding function.

As used throughout, the term “Cas13 polypeptide” means a Cas13 protein or a fragment thereof present in any bacterial species that encodes a Type VI CRISPR/Cas13 system. Examples include dPspCas13b, dLwaCas13a, and dRfxCas13d. See, for example, Abudayyeh et al., Science. 2016 Aug. 5; 353(6299): aaf5573. doi:10.1126/science.aaf5573, including supplemental information, hereby incorporated by reference in its entirety; Cox et al., Science 358, 1019-1027 (2017) including supplemental information, hereby incorporated by reference in its entirety; and Tang et al., Front. Cell Dev. Biol., 27 Jul. 2021 Sec. Epigenomics and Epigenetics Volume 9-2021|https://doi.org/10.3389/fcell.2021.677587. For example, the Cas13 protein or a fragment thereof with ssRNA targeting activity can be from Leptotrichia wadei, Leptotrichia shahii, Prevotella sp. P5-125 (PspCas13b), or Ruminococcus flavefaciens. Generally, Cas13 enzymes have two higher eukaryotes and prokaryotes nucleotide-binding (HEPN) endoRNase domains that mediate precise RNA cleavage with a preference for targets with protospacer flanking sites (PFSs) observed biochemically and in bacteria.

As used throughout, a dCas13 polypeptide is a deactivated or nuclease-dead Cas13 (dCas13) that has been modified to inactivate Cas13 nuclease activity. Modifications include, but are not limited to, altering one or more amino acids to inactivate the nuclease activity or the nuclease domain. For example, and not to be limiting, H133A and H1058A mutations can be made in Cas13 HEPN domains from Prevotella sp. P5-125 (PspCas13b) to inactivate Cas13 nuclease activity (see, for example, Cox et al., Science 358, 1019-1027 (2017) including supplemental information, hereby incorporated by reference in its entirety, and International Patent Publication WO 2019/005884, also incorporated by reference in its entirety). Other modifications include removing all or a portion of the nuclease domain of Cas13 (for example, Δ984-1090 H133A of Cas13b is from Prevotella sp. P5-125; see, for example, Programmable m(6)A modification of cellular RNAs with a Cas13-directed methyltransferase. Wilson C, Chen P J, Miao Z, Liu D R. Nat Biotechnol. 2020 Jun. 29. pii: 10.1038/s41587-020-0572-6. doi: 10.1038/s41587-020-0572-6. 10.1038/s41587-020-0572-6 PubMed 32601430), such that the sequences exhibiting nuclease activity are absent from Cas13. Additional mutations can include R474A/R1046A in dCas13 from L. wadei and mutations R239R/H244A/and R858A/H863A from Ruminococcus flavefaciens strain XPD3002. Accordingly, a dCas13 may include polypeptide sequences modified to inactivate nuclease activity or removal of a polypeptide sequence or sequences to inactivate nuclease activity. The dCas13 retains the ability to target ssRNA even though the nuclease activity has been inactivated. Accordingly, dCas13 includes the polypeptide sequence or sequences required for ssRNA targeting but includes modified nuclease sequences or lacks nuclease sequences responsible for nuclease activity.

In some examples, the dCas13 protein is a full-length Cas13 sequence from L. wadei, L. shahii, Prevotella sp. P5-125 (PspCas13b), or R. flavefaciens having one or more mutations in one or more HEPN domains and retaining the ssRNA targeting function. In other examples, the dCas13 protein sequences have at least 30%, 40%, 50%, 60%, 70%, 80%, 90%, 95%, 98% or 99% identity to Cas13 polypeptide sequences with HEPN mutations and retains RNA binding function

Utilizing these findings, described herein are novel therapeutics targeting or otherwise modulating SEL1L. Such novel therapeutics, compositions comprising novel therapeutics, methods of use, and kits can be useful, for example, for therapeutic administration to a subject.

### II. METHODS OF USE AND TREATMENT

Described herein are methods of use and methods of treatment regarding therapeutics described herein. In certain aspects, therapeutics can be utilized to treat SEL1L-HRD1 endoplasmic reticulum-associated degradation (ERAD) pathway disfunction, in particular those caused by mutations in Exon 4 of SEL1L. Methods as described herein can, for example, modulate SEL1L exon splicing as methods of treatment for rare disorders like ENDI and ENDI-A, for example, as well as more common conditions linked to ERAD dysfunction, including neurodevelopmental, metabolic, immune, and cancer-related diseases.

In an embodiment, described herein are methods of treating a SEL1L-associated ERAD-deficiency disorder in a subject having or suspected of having SEL1L-HRD1 ERAD pathway disfunction Methods described herein can comprise administering an antisense oligonucleotide to the subject in need thereof that induces skipping of SEL1L exon 4 (or any other exons), thereby restoring (or regulating) ERAD function to a subject in need thereof. In certain aspects, the subject in need thereof is a subject having or suspected of having ENDI and ENDI-A, for example, or another condition linked to ERAD dysfunction, including neurodevelopmental, metabolic, immune, and cancer-related diseases. In embodiments, the subject in need thereof has an SEL1L mutation, for example, an ENDI-A exon 4 mutation. In embodiments, the SEL1L mutation is C141Y. In embodiments, the treatment rescues one or more phenotypes selected from, for example: ERAD deficiency, perinatal lethality, or B-cell lymphopenia.

In embodiments, described herein are methods of treating a SEL1L loss-of-function mutation in a subject in need thereof. In embodiments, methods can comprise administering an adeno-associated virus (AAV) vector encoding SEL1L to increase SEL1L expression in the central nervous system and restore ERAD function. In embodiments, the loss-of-function mutation is the SEL1L mutation S658P. In embodiments, an AAV vector encoding SEL1L is administered intracerebroventricularly or intraparenchymally. In embodiments, the treatment improves any one or more of body weight or neurological symptoms.

In embodiments, described herein are methods of treating Alzheimer's disease in a subject having or suspected of having Alzheimer's disease. In embodiments, a method of treating Alzheimer's disease in a subject having or suspected of having Alzheimer's disease, comprises reducing SEL1L expression or activity in excitatory neurons, thereby decreasing Alzheimer's disease pathogenesis. In embodiments, SEL1L is reduced using RNA interference. In embodiments, SEL1L is reduced using an antisense oligonucleotide.

In embodiments of methods according to the present disclosure, a composition comprising a therapeutic as described herein (i.e., any one or more ASOs, AAVs, or RNAi polynucleotides) are administered in an amount effective to: rescues one or more phenotypes relating to ERAD deficiency, perinatal lethality, and/or B-cell lymphopenia; improve body weight; and/or improve any one or more neurological symptoms, for example, those present in subjects having or suspected of having Alzheimer's disease or other tauopathies.

In embodiments of AAV administration, a dose of about 10¹⁰ vg to about 10¹⁴ vg, or about 10¹² vg can be employed. A single dose or multiple doses can be utilized.

For splice switching ASO and gapmer ASOs, concentrations of about 8 μM to about 12 μM can be utilized. In embodiments, 10 μM can be utilized for 3 days. In other aspects, doses of about 10 mg/mL to about 15 mg/mL. In certain aspects, 12.5 mg/mL at a single dose for a week can be utilized.

In embodiments, splice switching ASOs and/or gapmer ASOs can be utilized for ENDI-A syndrome (specific for mutations in the SEL1L exon 4). This includes a group of symptoms of developmental delay, intellectual disability, B cell lymphopenia and agammaglobulinemia, axial hypotonia.

In embodiments, SEL1L inhibition can be utilized for subjects having, or suspected of having, Alzheimer's disease. In embodiments, SEL1L inhibition can improve one or more symptoms of Alzheimer's disease in the subject.

In embodiments, AAV-mediated overexpression can be utilized for ENDI syndrome, which can include a group of symptoms of developmental delay, intellectual disability, locomotor dysfunction, and/or facial dysmorphisms, for example. Methods according to the present disclosure can improve, reduce, or otherwise normalize any one or more symptoms of ENDI syndrome.

Additional aspects of subjects that can benefit from therapies described herein can be found, for example, in Wang, H. H., et al., Hypomorphic variants of SEL1L-HRD1 ER-associated degradation are associated with neurodevelopmental disorders. J Clin Invest, 2024. 134(2); and Weis, D., et al., Biallelic Cys141Tyr variant of SEL1L is associated with neurodevelopmental disorders, agammaglobulinemia, and premature death. J Clin Invest, 2024. 134(2), the entire contents of both of which are incorporated by reference as if fully set forth herein.

### III. KITS AND PACKAGING

Described herein are therapeutics and kits, as well as therapeutic kits. Kits can comprise any one or more therapeutics described herein.

In another embodiment, this kit comprises a (optionally sterile) solvent suitable for dissolving or suspending a composition of the presently disclosed subject matter prior to use.

Described herein are containers for storage and/or use, and instructions for use. The kit can be a package which houses a container which contains therapeutics of the disclosure or formulations of the disclosure and also houses instructions for administering the compounds or formulations for use. The disclosure further relates to a commercial package comprising therapeutics of the disclosure or formulations of the disclosure together with instructions for simultaneous, separate or sequential use. In particular a label may include amount, frequency, and method of use.

As used herein, an “instructional material” includes a publication, a recording, a diagram, or any other medium of expression which can be used to communicate the usefulness of the components thereof of the presently disclosed subject matter in the kit for protease digestion and therapeutic administration described herein. The instructional material of the kit of the presently disclosed subject matter may, for example, be affixed to a container which contains a composition of the presently disclosed subject matter or be shipped together with a container which contains the composition. Alternatively, the instructional material may be shipped separately from the container with the intention that the instructional material and the composition be used cooperatively by the recipient

The disclosure also relates to articles of manufacture and kits containing materials useful for using therapeutics disclosed herein. An article of manufacture may comprise a container with a label. Examples of suitable containers include bottles, vials, and test tubes, or a delivery device, which may be formed from a variety of materials including glass and plastic. A container holds therapeutics of the disclosure or formulations of the disclosure which are effective for treating a disease disclosed herein. The label on the container indicates that the therapeutics of the disclosure or formulations of the disclosure are used for applications disclosed herein and may also indicate directions for use.

The disclosure also contemplates kits comprising one or more therapeutics described herein for compositions of the disclosure. In aspects of the disclosure, a kit of the disclosure comprises a container described herein. In particular aspects, a kit of the disclosure comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein.

The compositions (i.e., those comprising, consisting essentially of, or consisting of enzymes described herein) can be utilized in the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the methods.

In some instances, one of the containers may comprise a composition as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. In some embodiments, the kit comprises separate containers containing compositions described herein and a detectable label.

In some embodiments, a kit comprises unit dose forms of a composition or components of compositions described herein.

In one embodiment, the kit comprises a composition as described herein in a defined, an effective dose in a single unit dosage form or as separate unit doses. The dose and form of the unit dose can be any doses or forms as described herein.

In certain embodiments, kits containing one or more containers of a formulation described in this disclosure are included

In an embodiment, a kit can comprise a therapeutic escribed herein, a polynucleotide described herein, a vector described herein, and/or a pharmaceutical composition described herein; and instructions for use.

In certain aspects, the therapeutic[s] described herein, polynucleotide described herein, vector described herein, and/or a pharmaceutical composition described herein can be provided in a dosage unit form.

### IV. PHARMACEUTICAL FORMULATIONS AND ROUTES OF ADMINISTRATION

Embodiments of the present disclosure include therapeutics (comprising one or more therapeutics described herein) as identified herein and formulated with one or more pharmaceutically acceptable excipients, diluents, carriers and/or adjuvants. In addition, embodiments of the present disclosure include a therapeutic formulated with one or more pharmaceutically acceptable auxiliary substances. In particular the therapeutic can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the therapeutics can be administered to the subject using any means capable of resulting in the desired effect. Thus, the therapeutics can be incorporated into a variety of formulations for therapeutic administration. For example, the therapeutics can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the therapeutics may be administered in the form of its pharmaceutically acceptable salts, or a subject active composition may be used alone or in appropriate association, as well as in combination, with other pharmaceutically active compounds. The following methods and excipients are merely exemplary and are in no way limiting.

For oral preparations, the therapeutic can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the therapeutics can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Embodiments of the compound can be utilized in aerosol formulation to be administered via inhalation. Embodiments of the compound can be formulated into pressurized acceptable propellants such as dichlorodifluoromethane, propane, nitrogen and the like.

Furthermore, embodiments of the compound can be made into suppositories by mixing with a variety of bases such as emulsifying bases or water-soluble bases. Embodiments of the compound can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compositions. Similarly, unit dosage forms for injection or intravenous administration may comprise the compound in a composition as a solution in sterile water, normal saline or another pharmaceutically acceptable carrier.

Embodiments of the therapeutics can be formulated in an injectable composition in accordance with the disclosure. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles in accordance with the present disclosure.

In an embodiment, the therapeutic can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of the compound can be accomplished using any of a variety of refillable, pump systems. Pumps provide consistent, controlled release over time. In some embodiments, the therapeutic can be in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852, and the like. In general, a subject treatment method can be accomplished using any of a variety of refillable, non-exchangeable pump systems. Pumps and other convective systems are generally preferred due to their generally more consistent, controlled release over time. Osmotic pumps are used in some embodiments due to their combined advantages of more consistent controlled release and relatively small size (see, e.g., PCT published application no. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

In some embodiments, an active agent (e.g., an ASO or other therapeutic described herein) can be delivered using an implantable drug delivery system, e.g., a system that is programmable to provide for administration of the agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present disclosure is the Synchromed infusion pump (Medtronic).

Suitable excipient vehicles for the compound are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the compound adequate to achieve the desired state in the subject being treated.

Compositions of the present disclosure can include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the pharmaceutical composition of the present disclosure (as well as combination compositions) can be delivered in a controlled release system. For example, the compound may be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) include those formed by impregnation of the compound described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

### A. Dosages

Embodiments of the compound can be administered to a subject in one or more doses. Those of skill will readily appreciate that dose levels can vary as a function of the specific the compound administered, the severity of the symptoms and the susceptibility of the subject to side effects. Preferred dosages for a given compound are readily determinable by those of skill in the art by a variety of means.

In an embodiment, multiple doses of the compound are administered. The frequency of administration of the compound can vary depending on any of a variety of factors, e.g., severity of the symptoms, and the like. For example, in an embodiment, the compound can be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). As discussed above, in an embodiment, the compound is administered continuously.

The duration of administration of the compound analogue, e.g., the period of time over which the compound is administered, can vary, depending on any of a variety of factors, e.g., patient response, etc. For example, the compound in combination or separately, can be administered over a period of time of about one day to one week, about two weeks to four weeks, about one month to two months, about two months to four months, about four months to six months, about six months to eight months, about eight months to 1 year, about 1 year to 2 years, or about 2 years to 4 years, or more.

### B. Routes of Administration

Embodiments of the present disclosure provide methods and compositions for the administration of the active agent (e.g., the compound) to a subject (e.g., a human) using any available method and route suitable for drug delivery, including in vivo and ex vivo methods, as well as systemic and localized routes of administration. For administration into the brain or CNS for example, routes include intracerebroventricularly or intraparenchymally.

Routes of administration include intranasal, intramuscular, intratracheal, subcutaneous, intradermal, topical application, intravenous, rectal, nasal, oral, and other enteral and parenteral routes of administration. Routes of administration may be combined, if desired, or adjusted depending upon the agent and/or the desired effect. An active agent (e.g., the compound) can be administered in a single dose or in multiple doses.

Embodiments of the compound can be administered to a subject using available conventional methods and routes suitable for delivery of conventional drugs, including systemic or localized routes. In general, routes of administration contemplated by the disclosure include, but are not limited to, enteral, parenteral, or inhalational routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the compound. Where systemic delivery is desired, administration typically involves invasive or systemically absorbed topical or mucosal administration of pharmaceutical preparations.

In an embodiment, the compound can also be delivered to the subject by enteral administration. Enteral routes of administration include, but are not limited to, oral and rectal (e.g., using a suppository) delivery.

Methods of administration of the compound through the skin or mucosa include, but are not limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” that deliver their product continuously via electric pulses through unbroken skin for periods of several days or more.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

Other features, objects, and advantages of the present invention are apparent in the description that follows. It should be understood, however, that the description, while exemplifying certain embodiments of the present invention, is given by way of illustration only, not limitation. Various changes and modifications within the scope of the invention will become apparent to those skilled in the art from the detailed description.

### V. EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1: Functional Rescue of a Fatal ERAD Mutation Via Alternative Splicing

Endoplasmic reticulum (ER)-associated degradation (ERAD) is essential for cellular proteostasis, with the SEL1L-HRD1 protein complex targeting misfolded proteins in the ER for proteasomal degradation. Disruption of this pathway underlies a recently identified infant-onset neurodevelopmental disorder (ENDI syndrome), characterized by profound developmental delay, microcephaly, and immune deficiency. Its most severe form, ENDI with agammaglobulinemia (ENDI-A), is driven by a bi-allelic SEL1L Cys141Tyr (C141Y) mutation within the fibronectin II (FNII) domain, for which no treatment currently exists. Here, it is serendipitously uncovered a striking mechanism of intrinsic rescue in knock-in mouse models of the C141Y mutation: enhanced usage of an alternative splice donor site within exon 4 bypasses the mutant FNII-encoding region, restoring ERAD activity and rescuing key disease phenotypes including perinatal lethality, growth retardation, B cell deficiency, and neurodevelopmental defects. Building on this discovery, it is demonstrated that antisense oligonucleotide (ASO)-mediated exon skipping in patient-derived fibroblasts generates a truncated yet functional SEL1L protein, fully rescuing ERAD function and ER proteostasis. These results establish RNA splicing modulation as a viable therapeutic strategy for ERAD deficiency and extend the clinical potential of exon-skipping therapy to diseases of protein misfolding.

### Discussion

Nascent proteins in the endoplasmic reticulum (ER) that fail to fold properly are recognized as misfolded and subsequently targeted for cytosolic proteasomal degradation by an ER quality control system known as ER-associated degradation (ERAD) (1-3). In mammals, the SEL1L-HRD1 protein complex represents the most conserved branch of ERAD (1, 4, 5). Misfolded proteins are first recognized by lectins such as OS9 and then delivered to SEL1L, which are then subsequently retrotranslocated through the HRD1 retrotranslocon followed by HRD1-mediated ubiquitination and proteasomal degradation (6-8). SEL1L is required for substrate recruitment, HRD1 protein stability (9, 10), and ERAD complex formation (11-13). Using various Sel1l- or Hrd1-knockout mouse models, it has been shown that the SEL1L-HRD1 ERAD pathway plays vital roles in a range of physiological processes in a substrate- and cell-type-specific manner, largely uncoupled from ER stress response (14-16).

The recent identification of human patients carrying bi-allelic SEL1L and HRD1 variants has provided direct evidence of its critical role in humans. To date, eleven patients have been found carrying four distinct bi-allelic hypomorphic variants in SEL1L and HRD1, all presenting with moderate to severe intellectual disability, microcephaly, developmental delay and locomotor dysfunction, collectively termed ENDI (17, 18). Among affected individuals, those carrying the SEL1L C141Y mutation (NM_005065.6: exon 4: c.422G>A, p.Cys141Tyr) exhibit not only the core symptoms of ENDI but also agammaglobulinemia and early mortality. This more severe phenotype, termed ENDI-agammaglobulinemia (ENDI-A), has been associated with heightened ERAD dysfunction (18). However, despite these insights, direct evidence supporting the pathogenicity of this variant remains limited, and no therapeutic strategies have been established to date.

In this study, while establishing the disease causality of the SEL1L C141Y mutation, it was unexpectedly discovered that modulating splicing at exon 4 could reverse disease pathology in a knock-in (KI) mouse model carrying the mutation. Building on this finding, ERAD function was successfully restored in SEL1L C141Y patient-derived human fibroblasts using splice-switching antisense oligonucleotide (ASO)-mediated exon skipping. While modulating alternative splicing using ASOs—short, synthetic nucleotides that bind target RNAs to alter splicing or promote degradation—has emerged as a powerful therapeutic approach for genetic diseases (19-21), these findings extend this strategy to target ERAD dysfunction, an area previously unexplored.

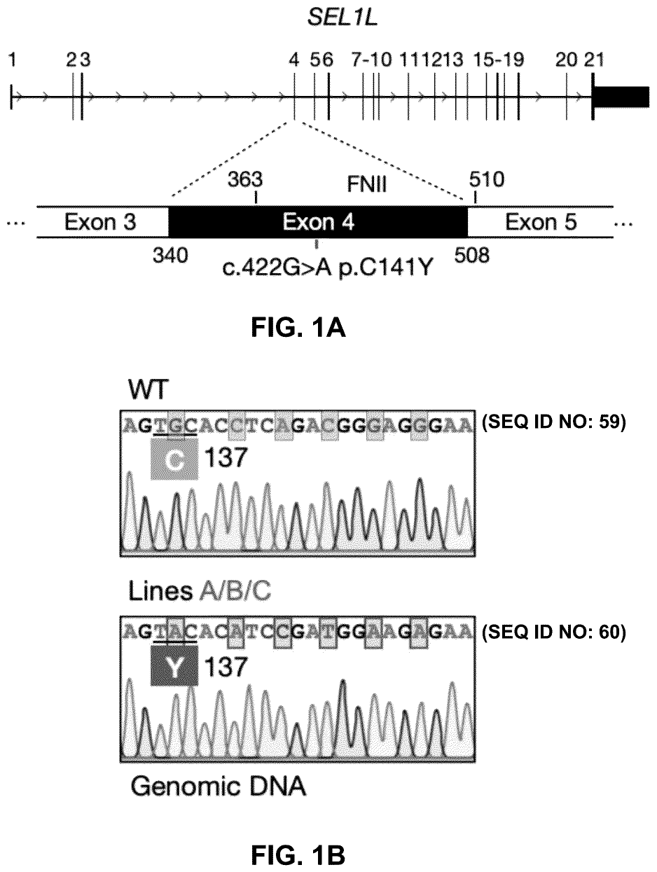

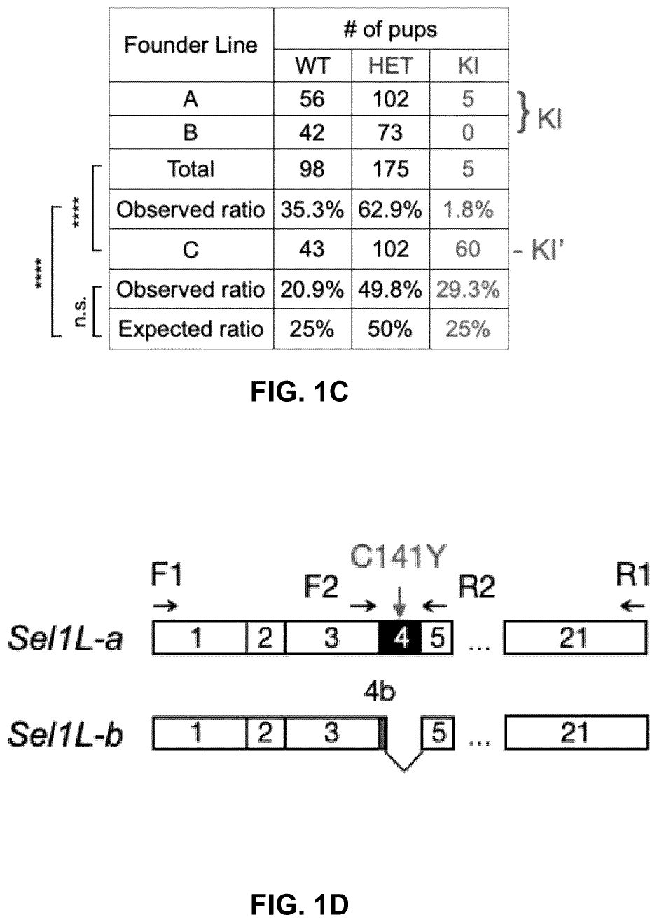

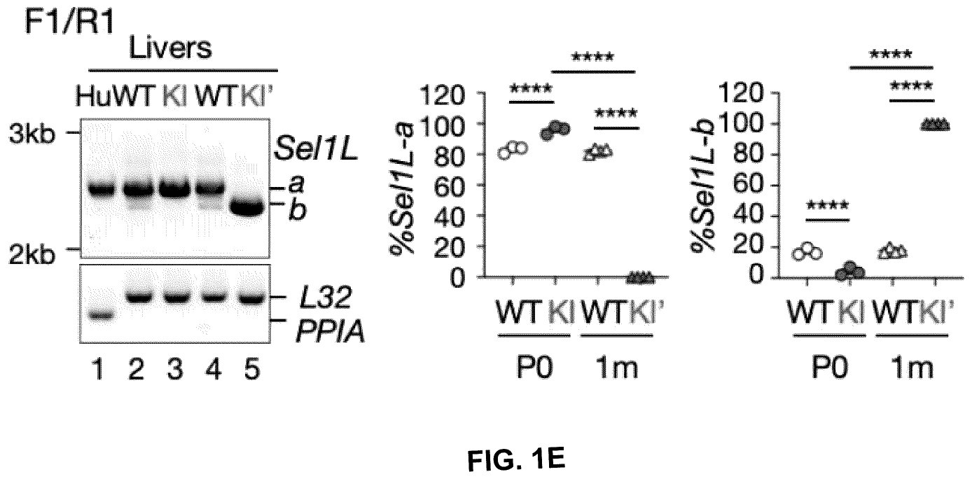

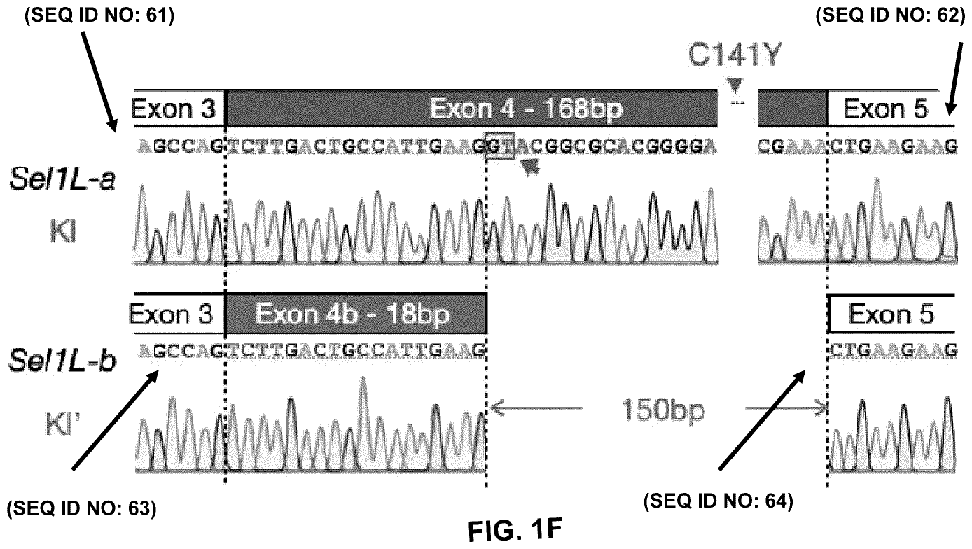

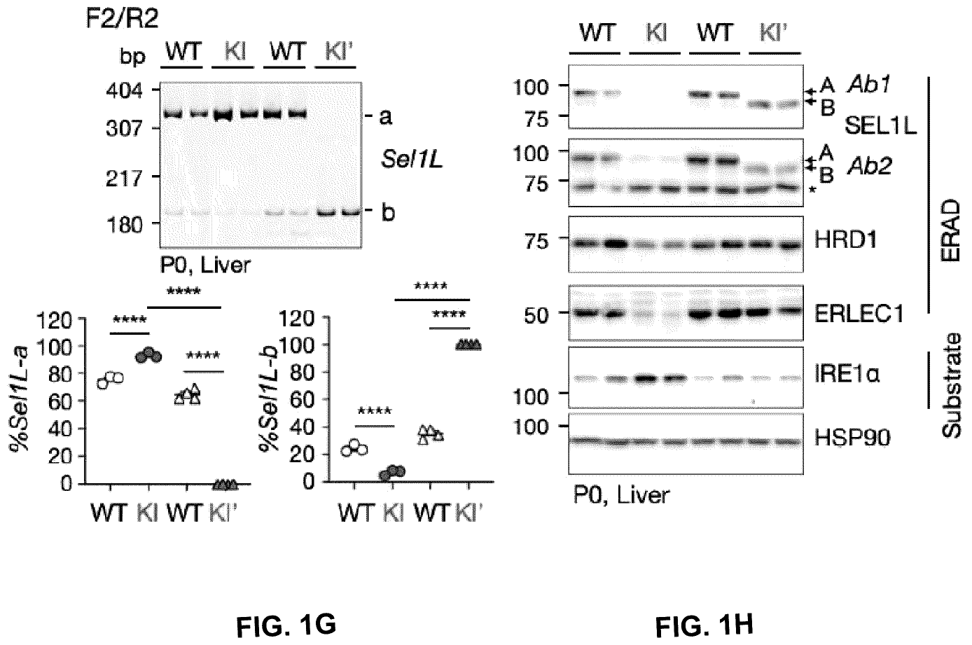

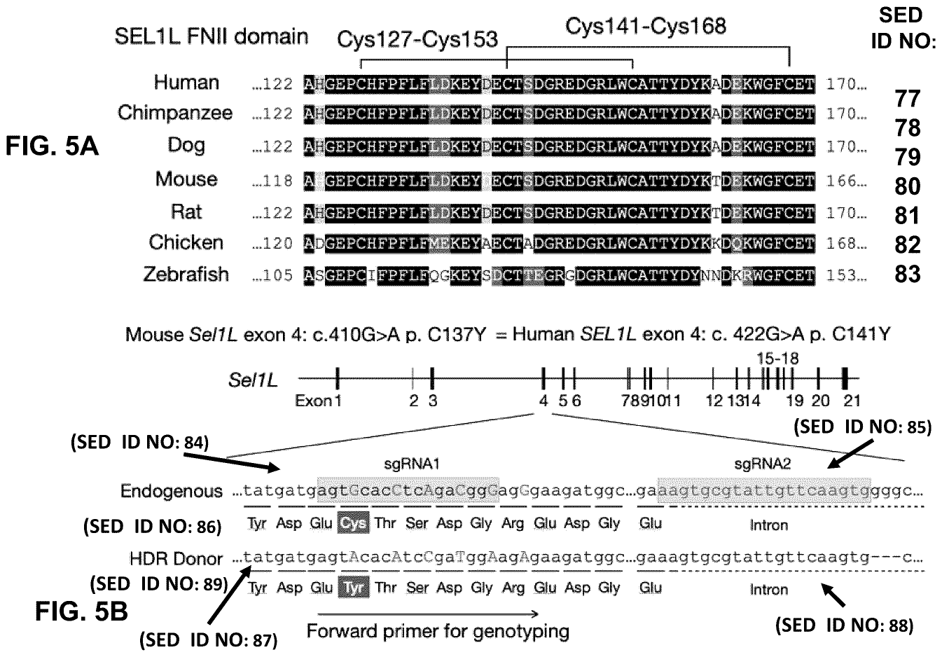

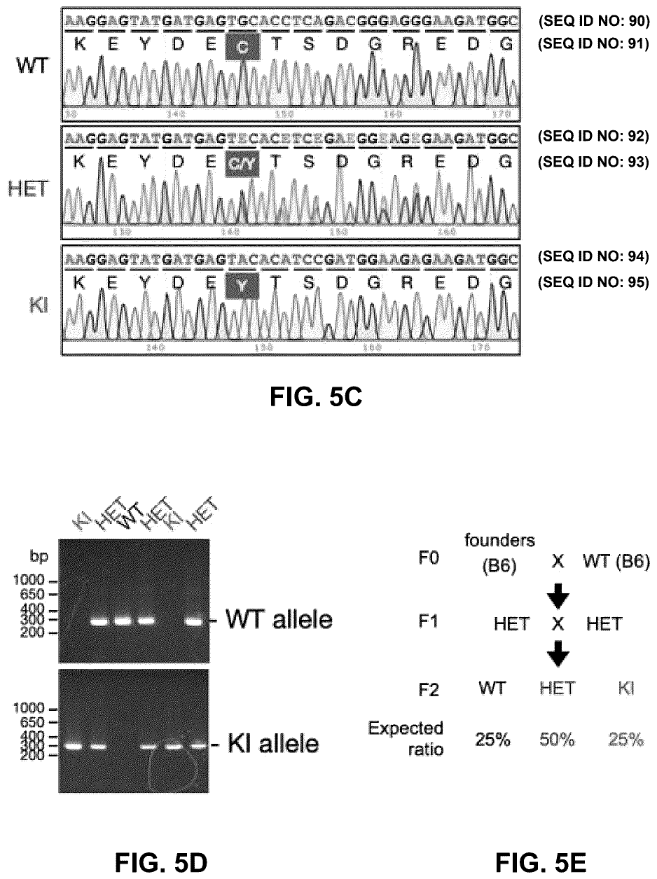

Alternative splicing of Sel1L exon 4 rescues lethality in SEL1L C141Y KI mice C141 is located within the fibronectin II (FNII) domain of SEL1L (FIG. 1A), a ˜50-amino acid motif of unknown function that is notably absent in yeast SEL1L ortholog Hrd3 (18, 22). This domain, encoded by exon 4 (FIG. 1A), is stabilized by two disulfide bonds (Cys127-Cys153 and Cys141-Cys168) (23), with C141 forming part of the latter (FIG. 5A). To investigate the disease causality and pathological consequences of SEL1L C141Y mutation (corresponding to C137Y in mice) in vivo, KI mouse models were generated using CRISPR-Cas9 genome editing (FIG. 5B). Sanger sequencing confirmed successful introduction of the intended G-to-A mutation in the genomes of three independent founder lines (Lines A-C) (FIG. 1B and FIG. 5C), from which were obtained WT, heterozygous (HET), and homozygous KI offspring (FIG. 5D to FIG. 5E). Unexpectedly, only Line C produced viable homozygous mice (hereafter KI′) at the expected ratio that survived past postnatal day 21 (p21) at Mendelian ratios. In contrast, Lines A and B yielded only 5 viable homozygous mice out of ˜280 pups surviving past weaning age p21—an overall survival rate of ˜1.8% (FIG. 1C). HET mice from all lines were viable (FIG. 1C) and appeared phenotypically indistinguishable from WT littermates. Given their shared phenotypes, they were grouped as the KI line for subsequent analyses using tissues from p0 neonates due to lethality.

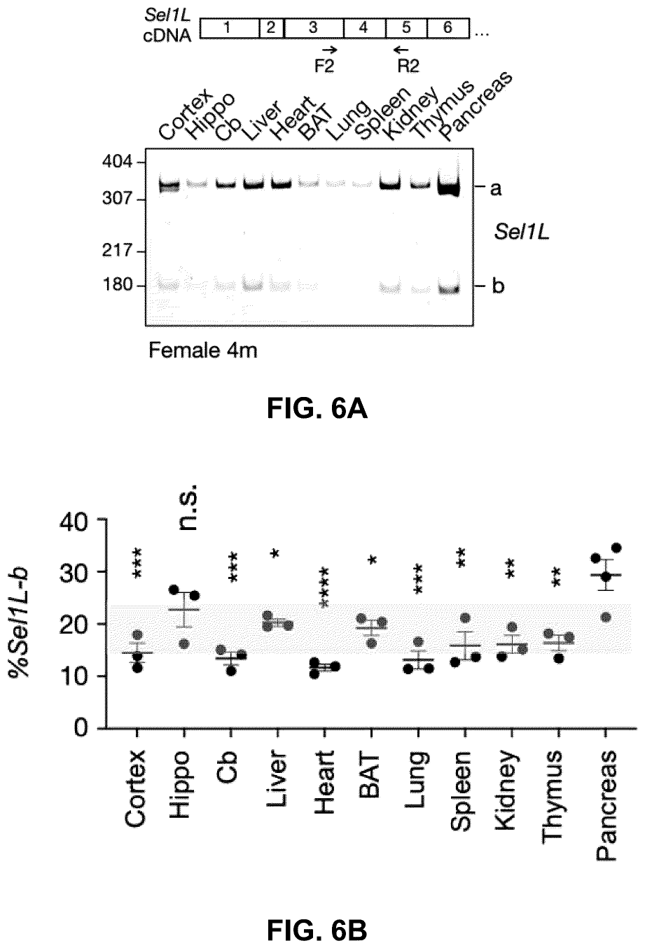

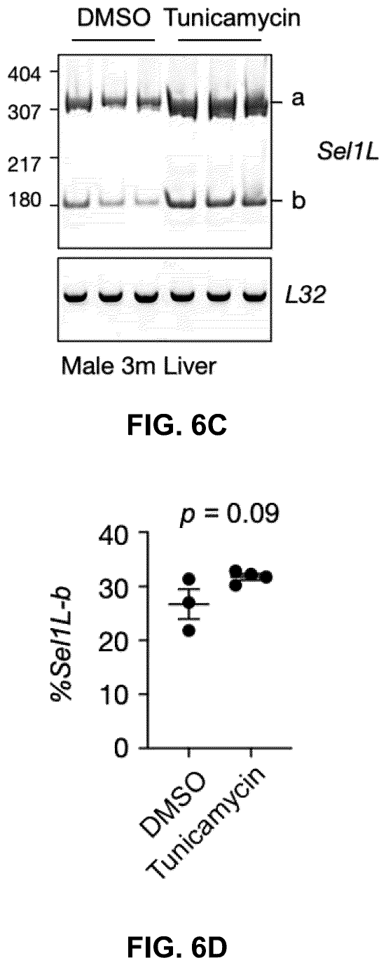

Interestingly, using the F1 R1 PCR primer set spanning all 21 exons of SEL1L, it was found that mouse Sel1L naturally expresses two mRNA isoforms in the liver (FIG. 1, D to E, Lanes 2 and 4). Sequencing revealed that these isoforms resulted from alternative splicing of exon 4: a predominant full-length isoform (Sel1L-a, ˜80%), and a shorter isoform (Sel1L-b) lacking 150 bp due to the use of an alternative “GU” splice donor site within exon 4 (arrow, FIG. 1F; illustrated in FIG. 1D). This splicing event removed amino acids 118-166, eliminating most of the FNII domain, including the C141Y mutation site (FIG. 1F). Notably, using the F2R2 primer set flanking exon 4 (FIG. 6A), it was found that despite large variations in overall Sel1L expression levels across mouse tissues (for example, a 5-fold difference between pancreas and spleen or lungs), the relative abundance of isoforms a and b remained consistent, with isoform b comprising ˜20% of total Sel1L transcript (FIG. 6A to FIG. 6B). Moreover, this splice pattern in the liver was unaffected by ER stress induced by tunicamycin injection (FIG. 6C to FIG. 6D).

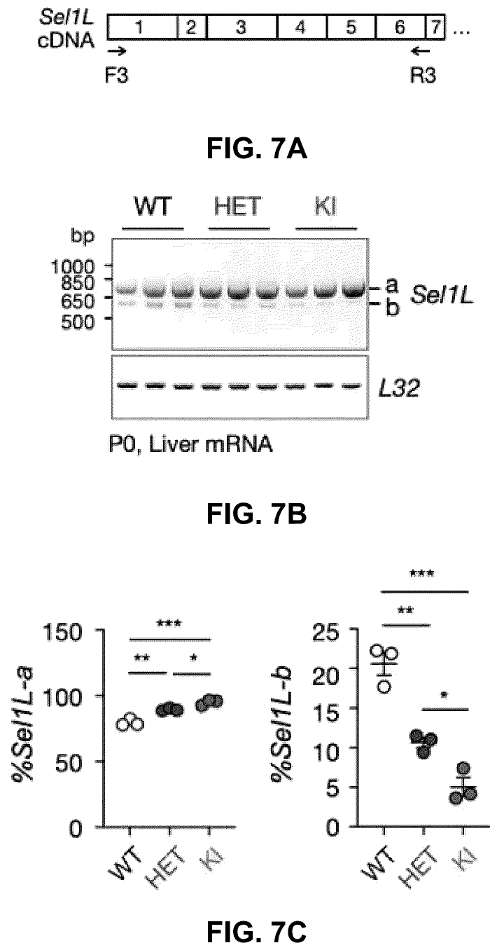

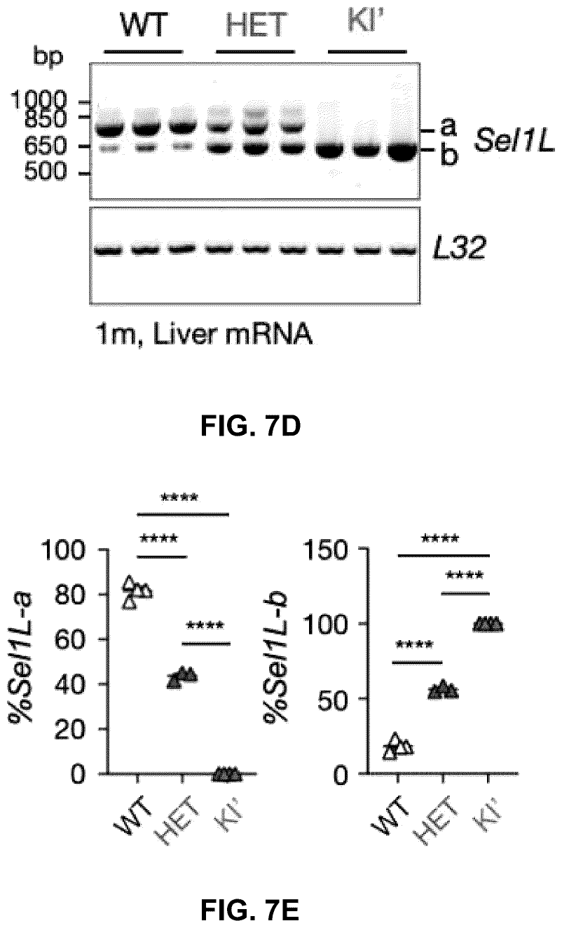

Sequence analysis confirmed that no additional mutations were present in the Sel1L cDNA aside from the engineered C141Y mutation (data not shown). Remarkably, KI mice predominantly expressed the full-length Sel1L-a isoform, while the independently generated KI′ line primarily expressed shorter Sel1L-b isoform (FIG. 1E). In the KI′ line, the alternative splicing occurred at the same internal alternative “GU” splice donor site within exon 4, thereby excluding the pathogenic C141Y mutation (FIG. 1F). To further confirm this splicing pattern, RT-PCR was performed using the alternative primer pair flanking exon 4 (F2R2, FIG. 1D) and products were resolved via acrylamide gel electrophoresis to enhance separation of small fragments (FIG. 1G). While WT livers showed a 4:1 ratio of Sel1L-a to -b, KI and KI′ livers exclusively expressed Sel1L-a and Sel1L-b, respectively (FIG. 1G). Using an additional primer pair F3R3 spanning exons 1 and 6 (FIG. 7A), it was further confirmed the splicing patterns observed above (FIG. 7B to FIG. 7E). Notably, HET mice from both lines displayed intermediate isoform expression levels, falling between those of WT and the respective KI lines (FIG. 7B to FIG. 7E).

### Alternative Splicing of Sel1L Exon 4 Rescues ERAD Defects in KI′ Mice

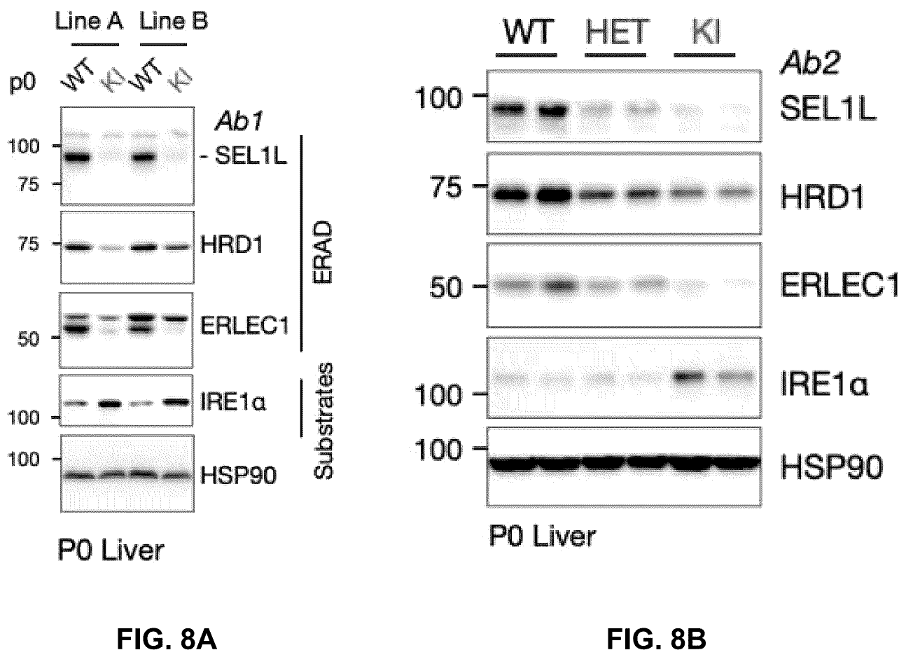

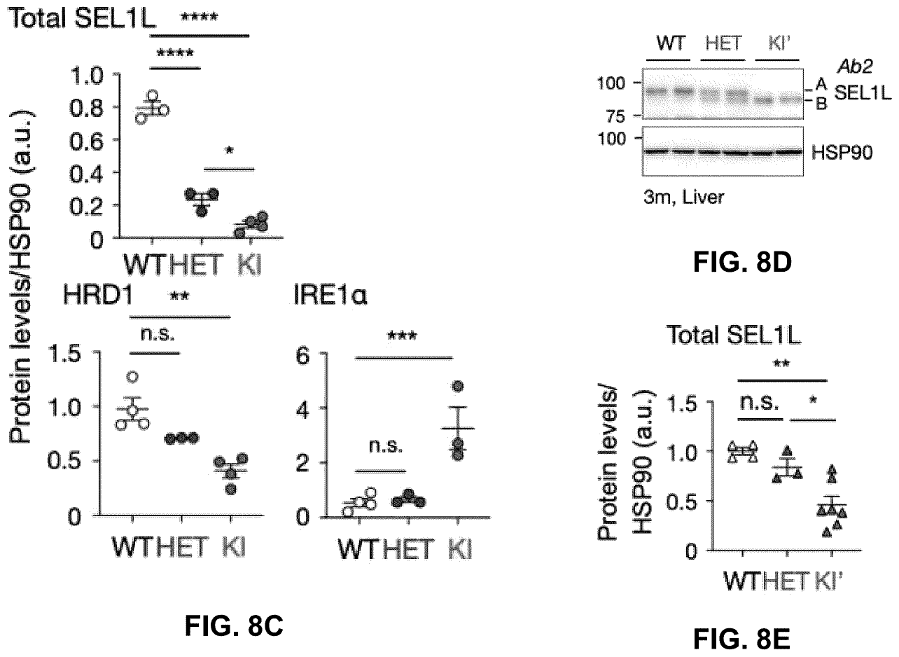

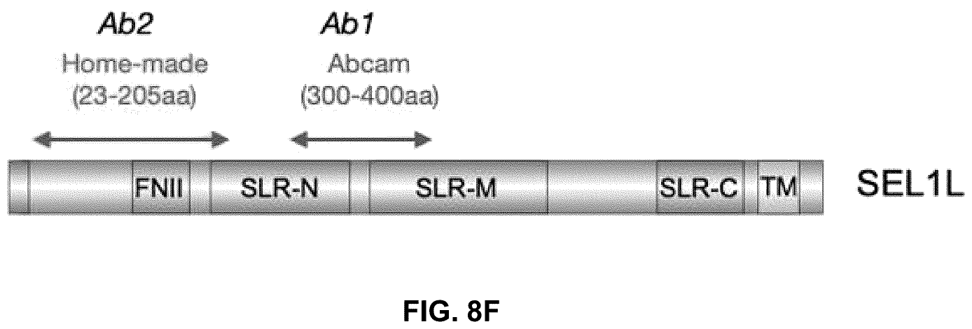

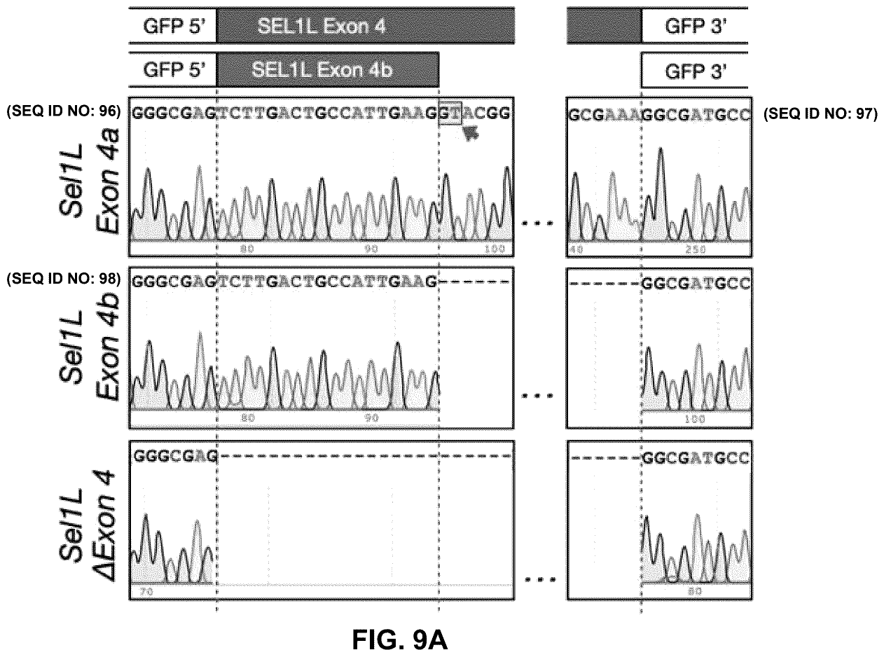

At the protein level, KI livers exhibited a near-complete loss of SEL1L expression, along with ˜60% reduction in the E3 ligase HRD1, ˜80% decrease in the ER lectin ERLEC1, and a ˜4-fold accumulation of the ERAD substrate IRE1α (24), consistent with ERAD dysfunction (FIG. 1H to FIG. 1I and FIG. 8A FIG. 8C). In contrast, KI′ livers restored SEL1L expression to ˜50% of WT levels despite harboring the same Sel1L C141Y mutation at the genomic level (FIG. 1B). These mice expressed a truncated SEL1L-B protein, ˜5 kDa smaller than the full-length isoform A, consistent with exon 4 skipping (FIG. 1H to FIG. 1I and FIG. 8D to FIG. 8E). This was confirmed using two independent antibodies targeting distinct SEL1L epitopes (18) (FIG. 1H and FIG. 8F). Expression of truncated SEL1L-B protein at ˜50% of WT levels was sufficient to restore HRD1 and ERLEC1 expression and to prevent IRE1a accumulation (FIG. 1H to FIG. 1I), indicating preserved ERAD function in KI′ livers. HET mice from both lines showed intermediate SEL1L isoform expression between WT and their respective KI counterparts (FIG. 8B to FIG. 8E), consistent with a recessive inheritance model (18). Taken together, these findings demonstrate that alternative splicing of Sel1L exon 4 effectively rescues both ERAD defects and the lethality caused by the C141Y mutation in vivo.

### Disruption of the Intronic Splice Donor Enables Alternative Splicing in KI′ Mice

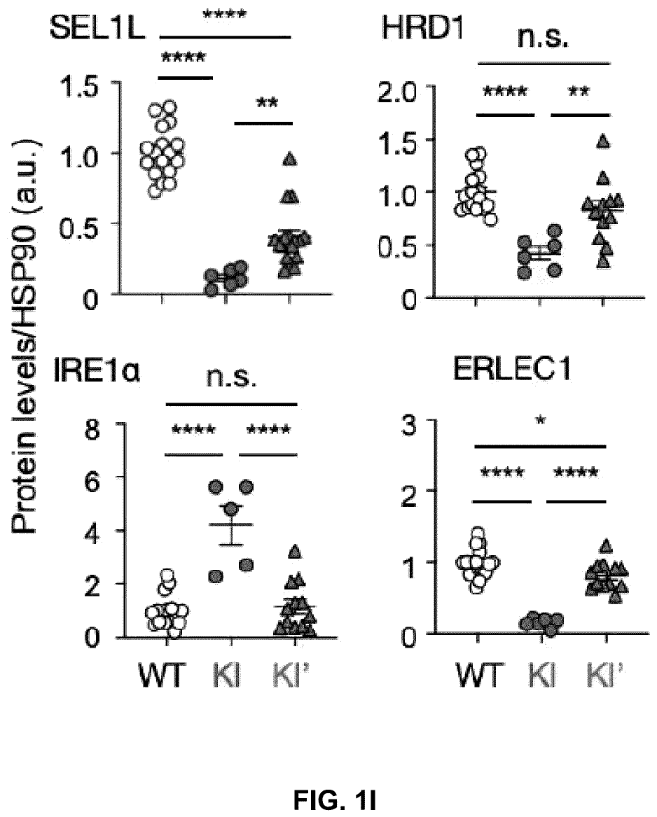

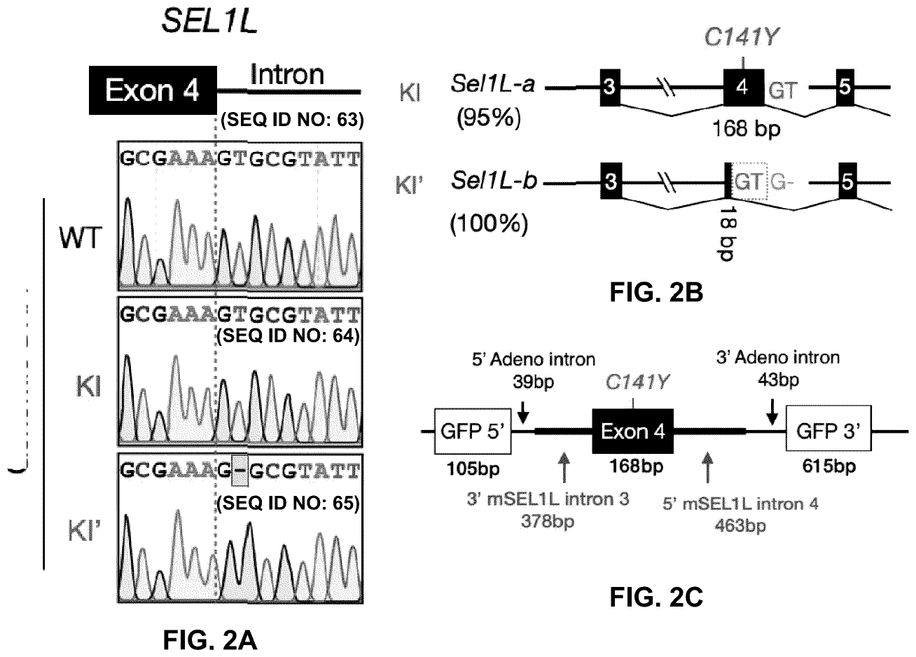

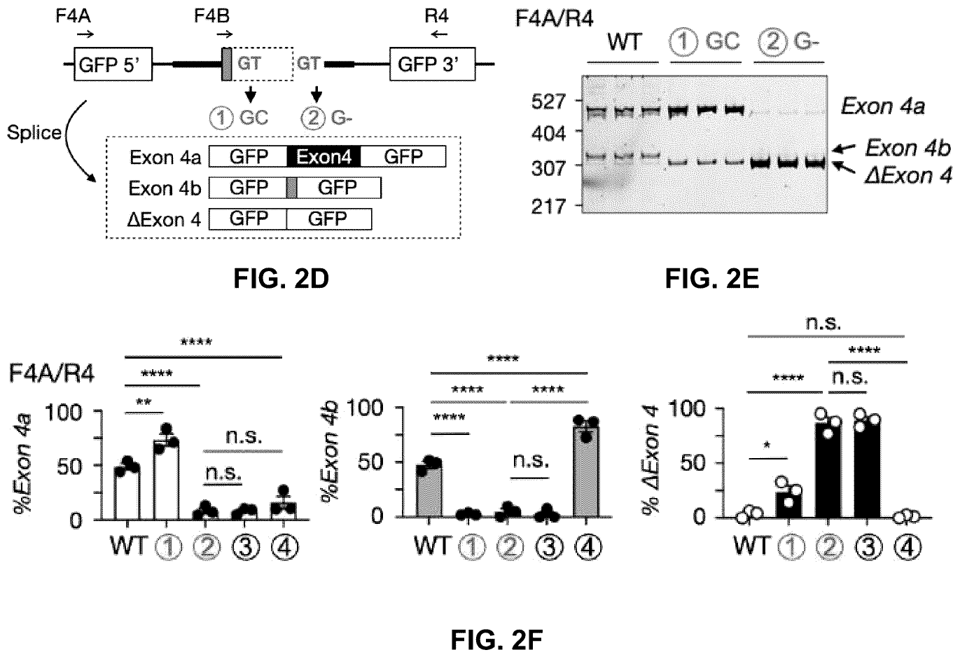

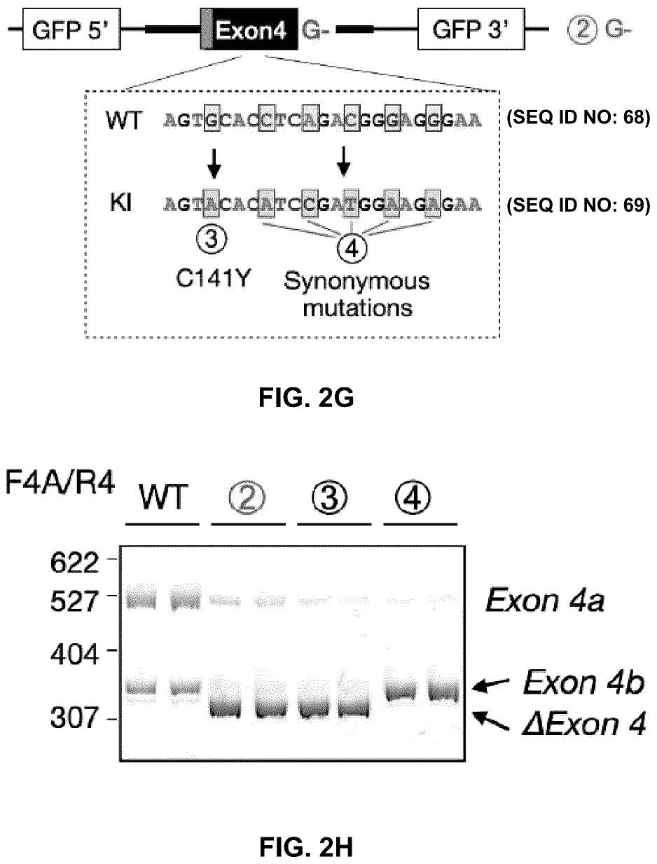

To investigate the mechanism underlying the preferential use of an alternative splicing donor site within exon 4 in KI′ mice, the genome region surrounding Sel1L exon 4 was sequenced. This analysis identified a single-nucleotide (T) deletion at the canonical “GT” splice donor site at the exon 4-intron 4 junction (FIG. 2A), likely introduced during the CRISPR/Cas9-mediated gene editing. To determine whether this disruption alone was sufficient to promote alternative splicing (FIG. 2B), a splicing minigene reporter (25) was constructed containing 168 bp exon 4 flanked by segments of 378 bp introns 3 and 463 bp intron 4, inserted between two GFP fragments (FIG. 2C).

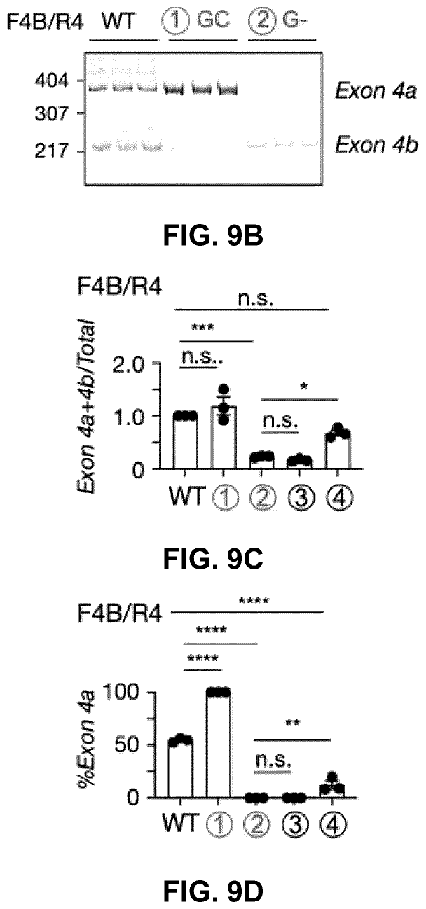

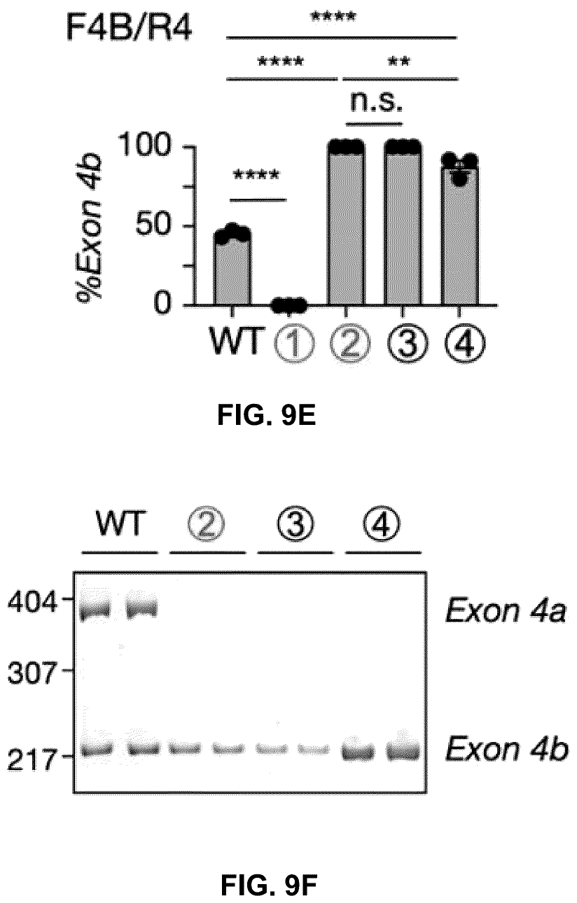

RT-PCR using the F4A/R4 primer pair on GFP (FIG. 2D) revealed that the minigene containing WT SEL1L sequence yielded both full-length exon 4 and truncated exon 4b isoforms, consistent with utilization of both canonical and internal alternative splice donor sites (FIG. 2E, quantitated in FIG. 2F; sequencing data in FIG. 9A). Mutation of the internal alternative splice donor site (mutation #1, GT→GC) abolished production of the exon 4b isoform, confirming its functionality (FIG. 2E, quantitated in FIG. 2F). Surprisingly, deletion of the canonical splice donor site (mutation #2, GT→G-) resulted in skipping of the entire exon 4 (ΔExon 4), rather than increase usage of the internal alternative site (FIG. 2E, quantitated in FIG. 2F). This result was corroborated using the F4B/R4 primer pair (FIG. 2D), which excludes the ΔExon 4 product, and revealed minimal expression of the exon 4b isoform under these conditions (FIG. 9B to 9C).

This unexpected outcome prompted the investigation of whether additional sequence changes introduced during CRISPR/Cas9 editing—including the C141Y mutation and nearby synonymous mutations used for genotyping (FIG. 2G)—influence splice site selection. Either the C141Y mutation (mutation #3, G→A) or the synonymous substitutions (mutation #4, C..A..C..G..G→A..C..T..A..A) were introduced into the minigene carrying mutation #2, which disrupts the canonical splice donor site (FIG. 2G). While the C141Y mutation did not alter the splicing pattern, the synonymous changes significantly enhanced usage of the alternative splice donor, resulting in increased expression of the exon 4b isoform (FIG. 2H, quantitated in FIG. 2F). These findings were further validated using F4B/R4 primer set (FIG. 9B to FIG. 9E). Together, these results suggest that loss of the canonical splice donor site creates a permissive context for exon skipping, while nearby exonic mutations—including synonymous changes—can promote use of an alternative splice donor site, likely by modulating exonic splicing regulatory elements.

### Pathophysiological Significance of Sel1L Alternative Splicing

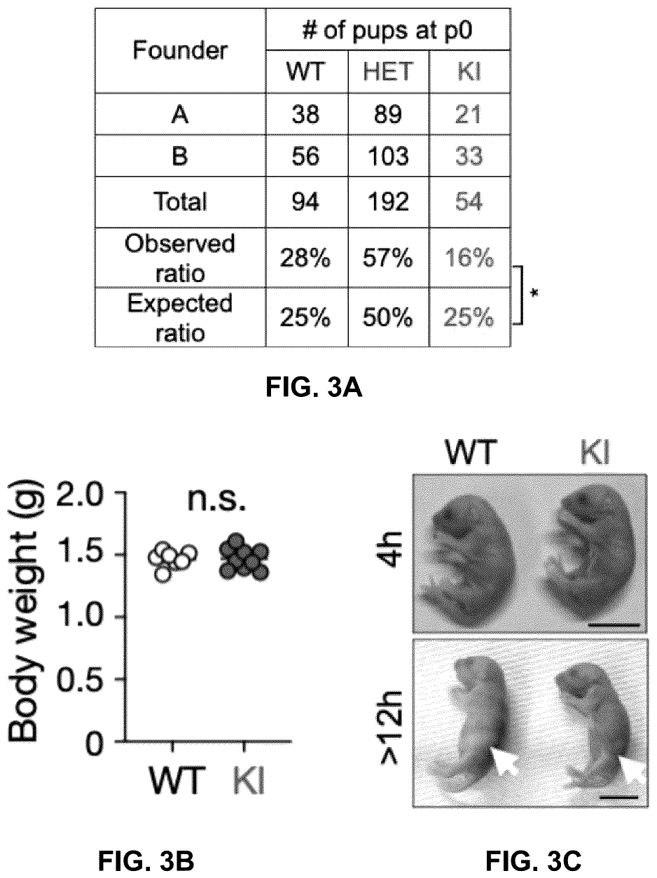

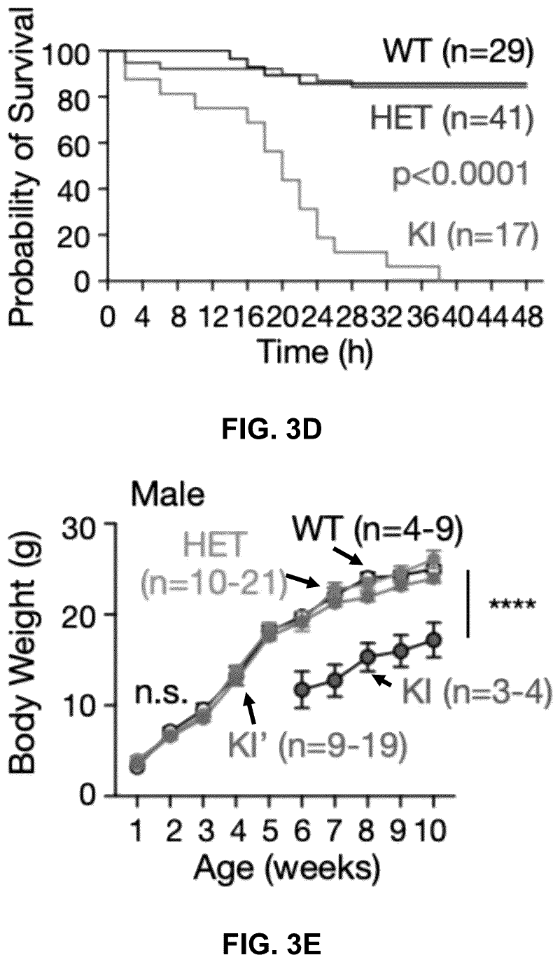

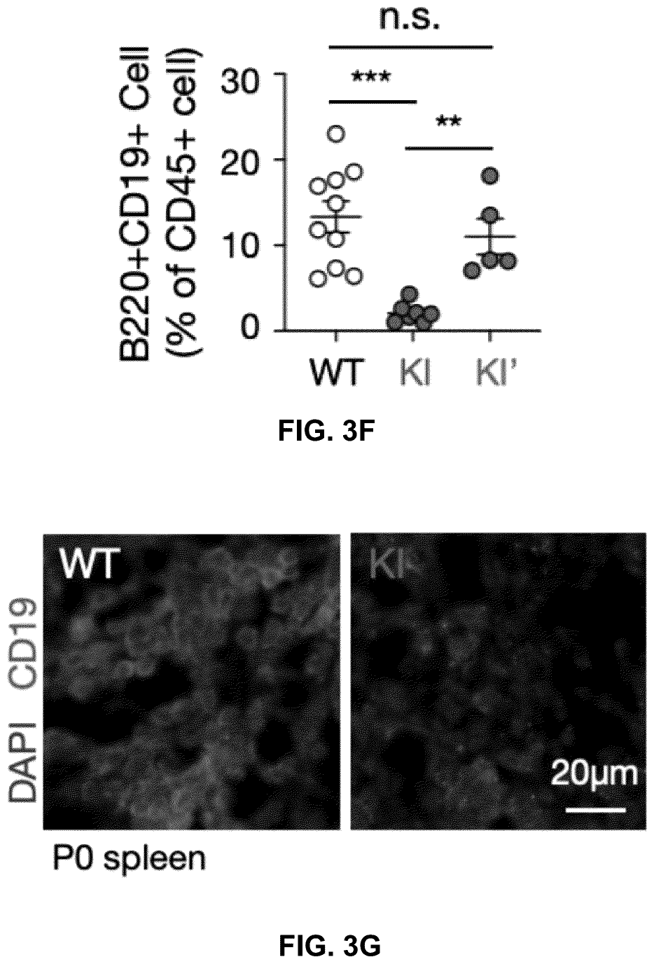

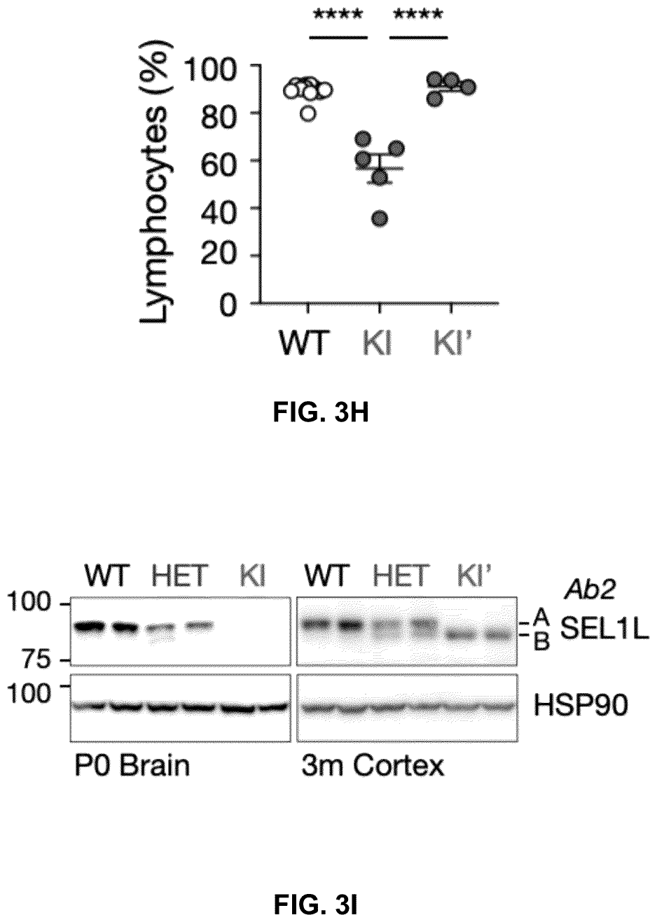

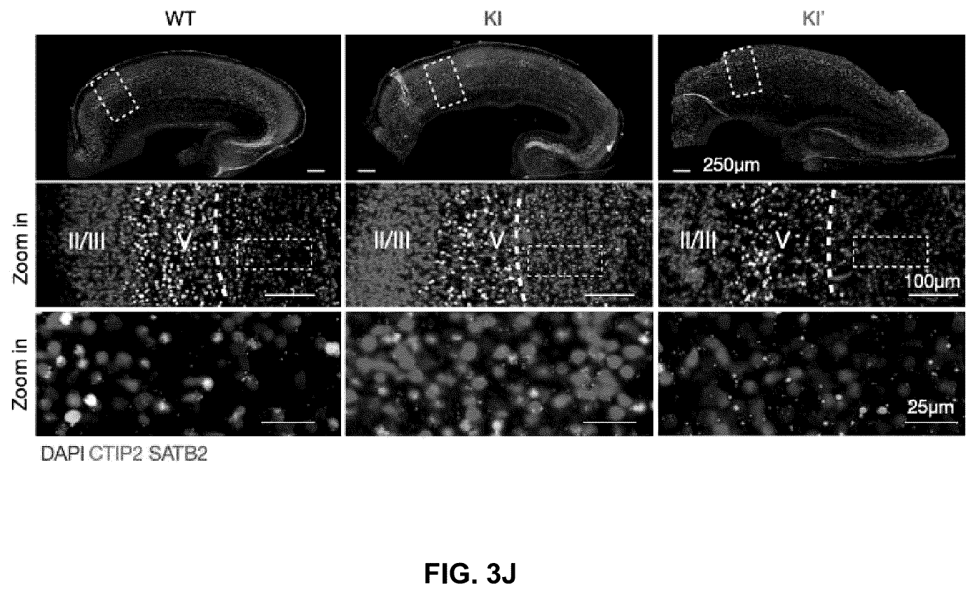

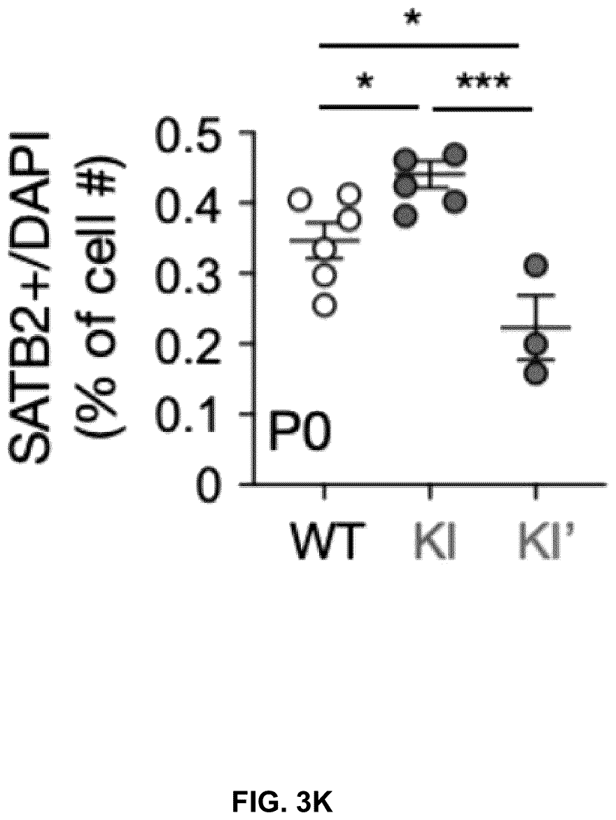

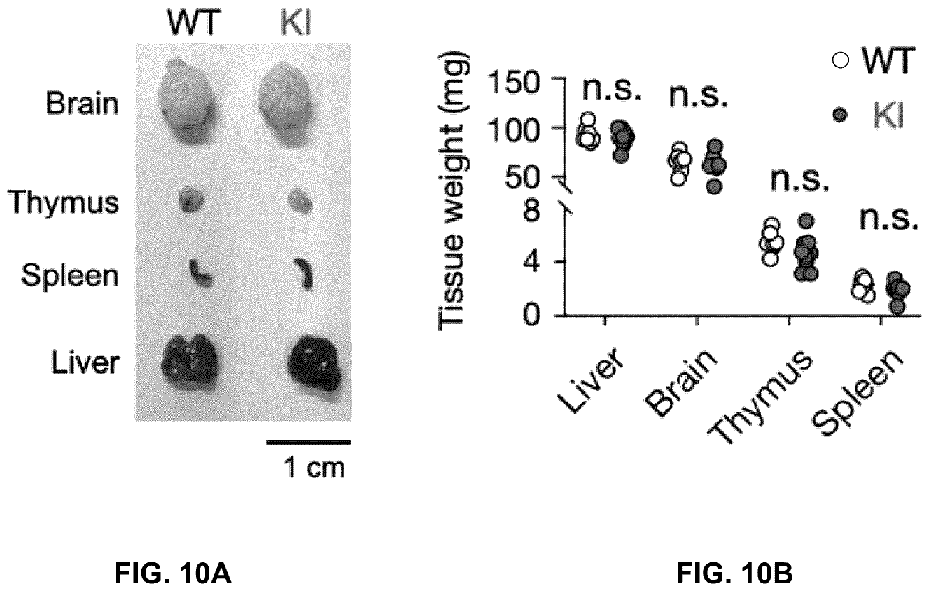

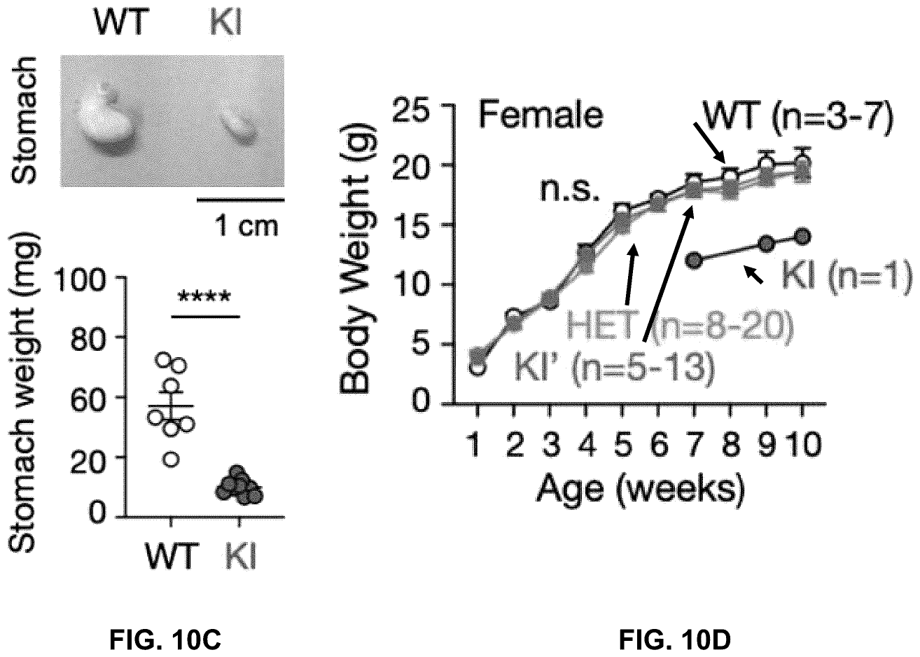

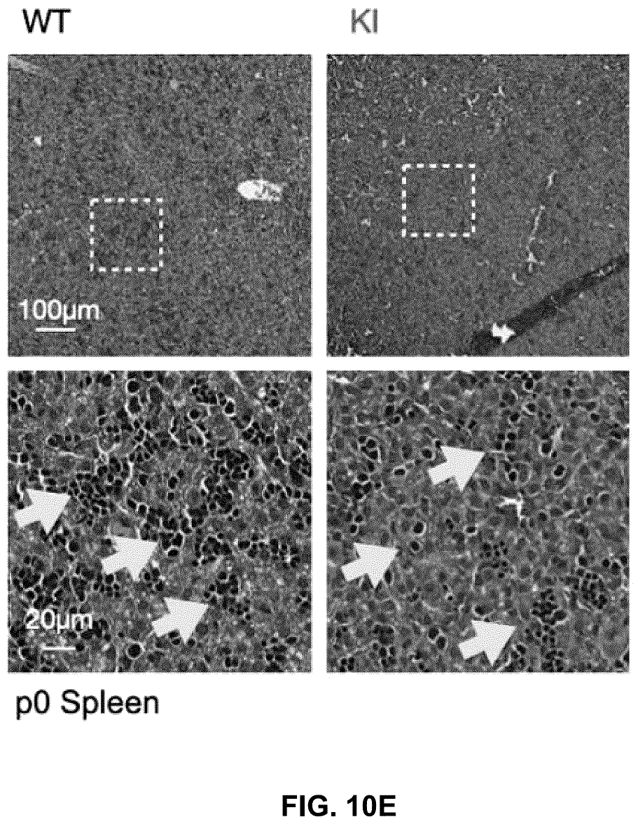

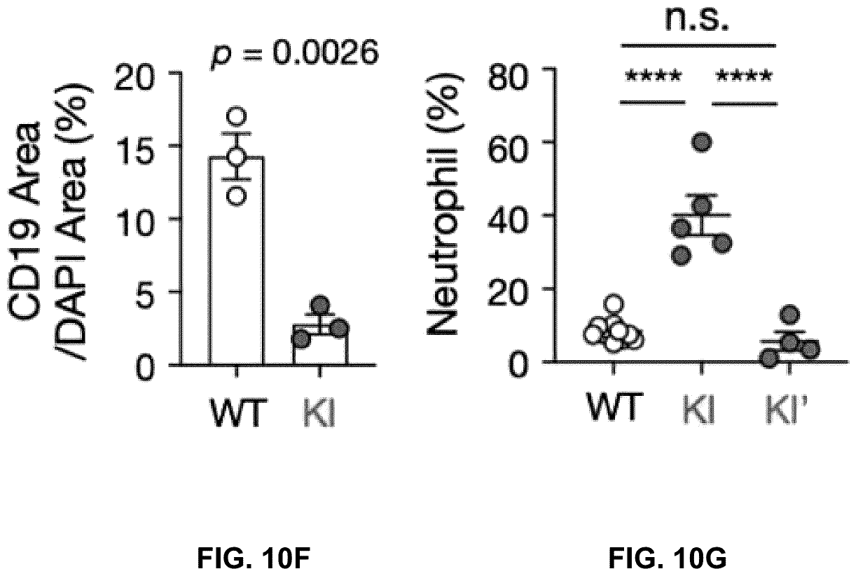

To assess the physiological relevance of Sel1L alternative splicing, KI and KI′ mice were phenotypically characterized and compared. While very few KI pups could survive past weaning (FIG. 1C), they were born—indicating that the mutation was not embryonic lethal—albeit at a reduced frequency of 16% at p0 (FIG. 3A). At birth, KI pups were indistinguishable from WT littermates in body weight (FIG. 3B), gross morphology (FIG. 3C), and tissue mass (FIG. 10A to FIG. 10B). However, by 12 hours postpartum, KI neonates lacked visible milk sacs (arrows, FIG. 3C and FIG. 10C), indicative of impaired feeding. These phenotype parallels clinical reports of severe feeding difficulties in ENDI-A patients shortly after birth (18). Within the defined cohort, all KI pups died within 48 hours of birth (FIG. 3D), although rare survivors were observed in separate litters (5 out of 290 pups, FIG. 1C). Among these, 5 mice (4 males, 1 female) exhibited profound postnatal growth retardation (FIG. 3E and FIG. 10D), mirroring the clinical presentation of ENDI-A (18). In contrast, KI′ mice were born at expected Mendelian ratios and showed normal growth comparable to WT and HET littermates (FIG. 3E and FIG. 10D).

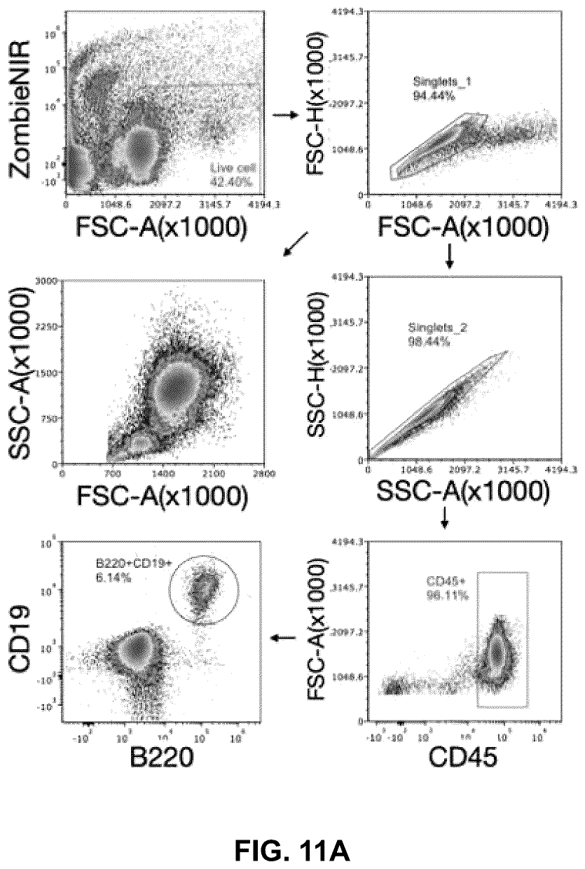

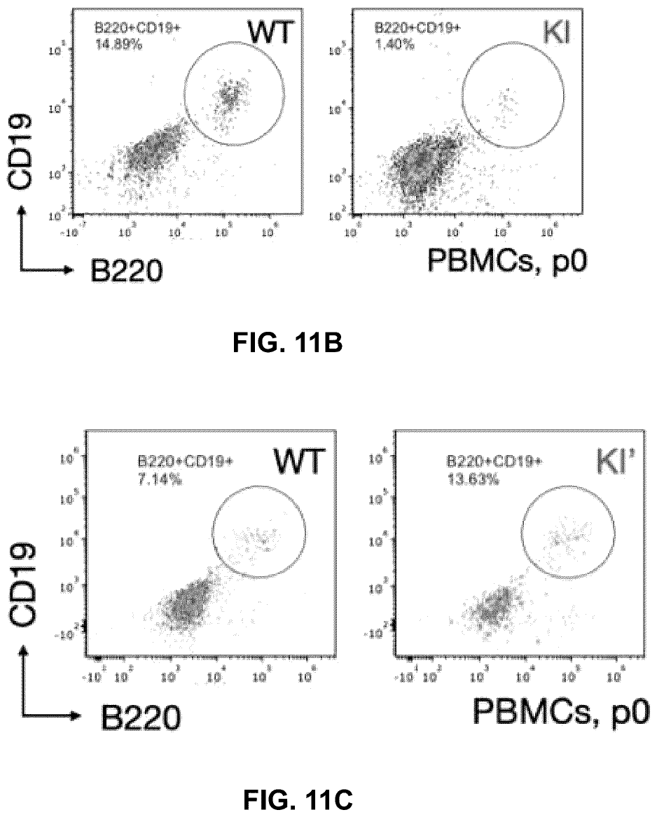

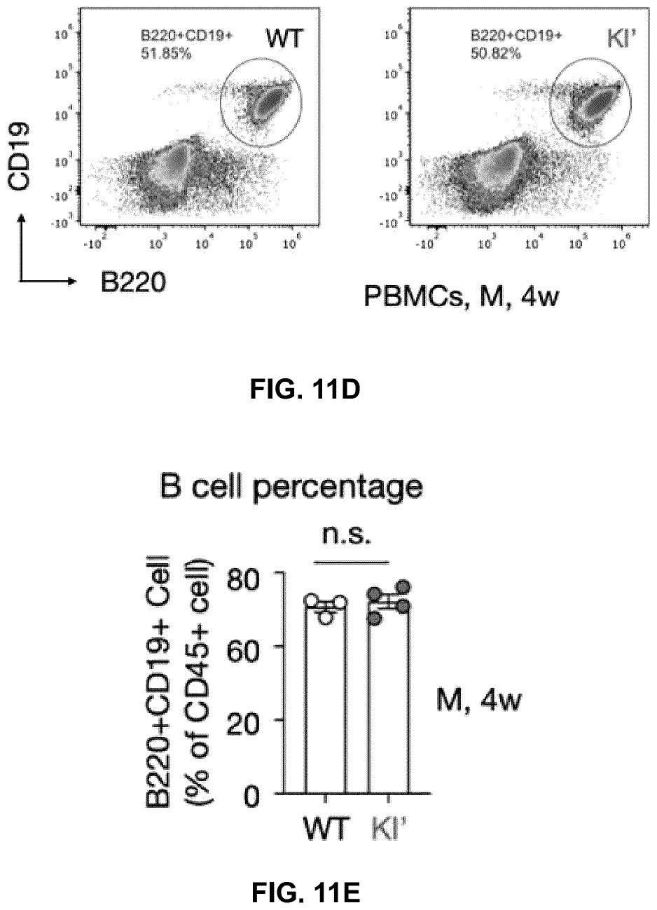

ENDI-A patients exhibit profound B cell lymphopenia (18), akin to mice with B cell-specific deletion of Sel1L (26). Similarly, KI neonates exhibited marked reductions in mature B cells in peripheral blood and spleen at p0, as measured by flow cytometry (FIG. 3F and FIG. 11A to FIG. 11B), histology (arrows, FIG. 10E), and immunofluorescence staining using mature B cell markers CD19 and B220 (FIG. 3G, and FIG. 10F). Surviving adult KI mice also showed significantly reduced lymphocytes in peripheral blood mononuclear cells (PBMCs) (FIG. 3H) and a notable expansion of circulating neutrophils (FIG. 10G), a phenotype commonly observed in B cell-deficient models (27). By contrast, KI′ mice showed normal circulating B cell numbers at birth (FIG. 3F and FIG. 11C) and puberty (FIG. 11D to FIG. 11E), with no elevation in neutrophils (FIG. 10G).

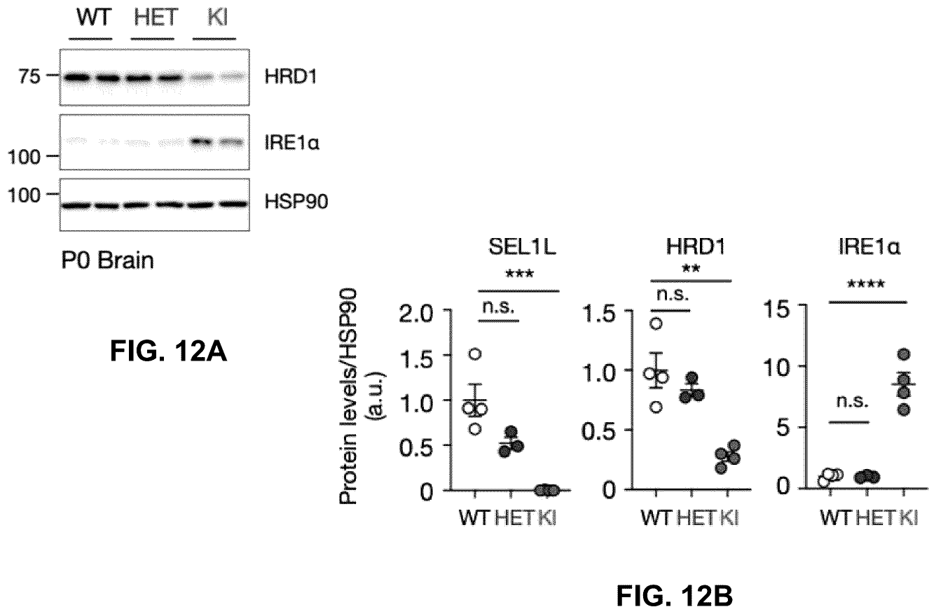

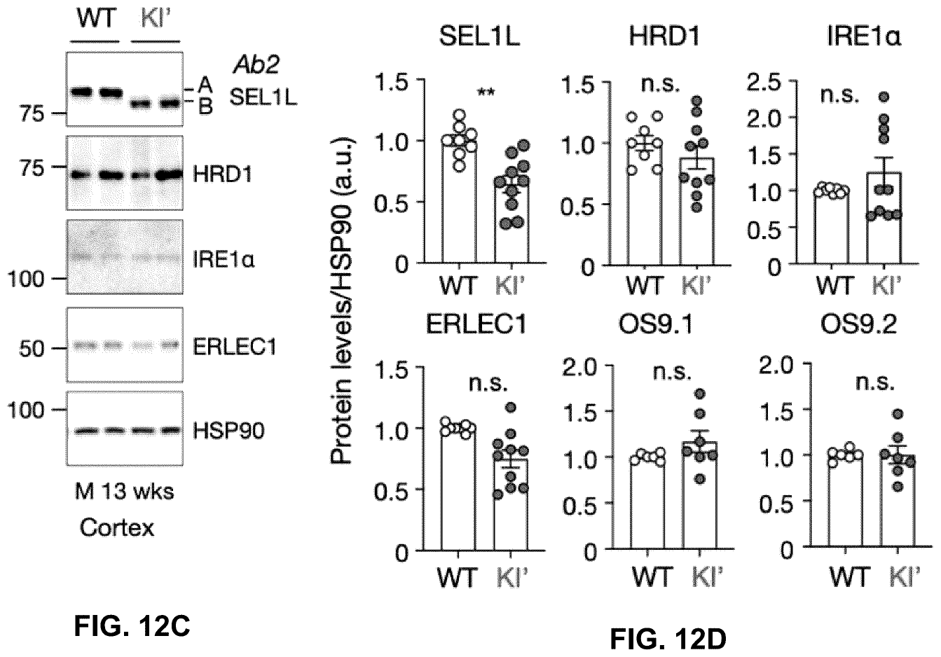

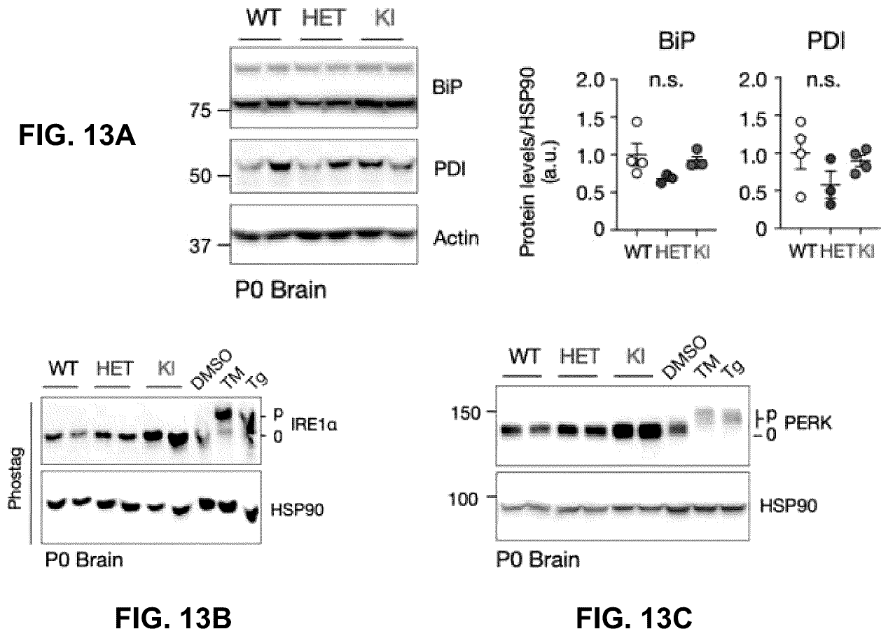

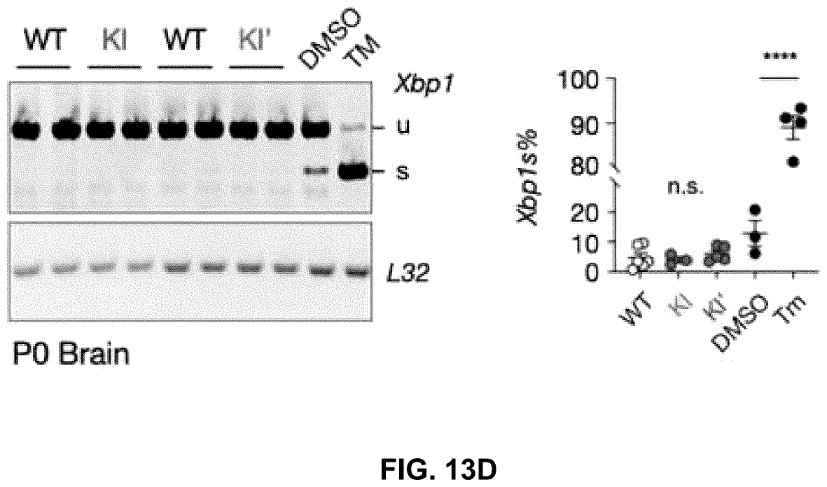

Given that ENDI patients also present with intellectual disability (17, 18), cortical development at p0 was examined. As observed in the liver (FIG. 1H), KI brains showed near-complete loss of SEL1L expression, reduced HRD1 protein levels (by ˜70%), and ERAD dysfunction—all of which were restored in KI′ mice (FIG. 3I, FIG. S8, FIG. 12A to FIG. 12D). Cortical development occurs during embryogenesis and results in the formation of six distinct layers arranged in an inside-out pattern from Layer VI to Layer I (28). In WT brains, immunofluorescence staining revealed well-organized cortical layering, with clearly defined Layer II/III (SATB2-positive) and Layer V (CTIP2-positive) neurons (FIG. 3, J to K). In contrast, KI mice exhibited disrupted cortical architecture, including ectopic localization of SATB2-positive cells below Layer V without obvious cell loss, indicative of impaired neuronal migration or cortical specification (FIG. 3, J to K). Interestingly, KI′ mice displayed a normalized SATB2+ cell distribution and restored laminar organization (FIG. 3, J to K). Moreover, minimal UPR activation was observed as indicated by the absence of ER chaperone induction (BiP and PDI, FIG. 13A) and lack of UPR sensor activation, including IRE1α and PERK phosphorylation and Xbp1 mRNA splicing, in the brains of KI pups (FIG. 13B to FIG. 13D). Collectively, these findings establish pathogenic nature of the SEL1L C141Y variant and demonstrate that exon 4 skipping via alternative splicing functionally rescue the lethal and developmental defects associated with the mutation in vivo.

### ASO-Mediated Exon Skipping Rescues ERAD Defects in Patient-Derived Fibroblasts

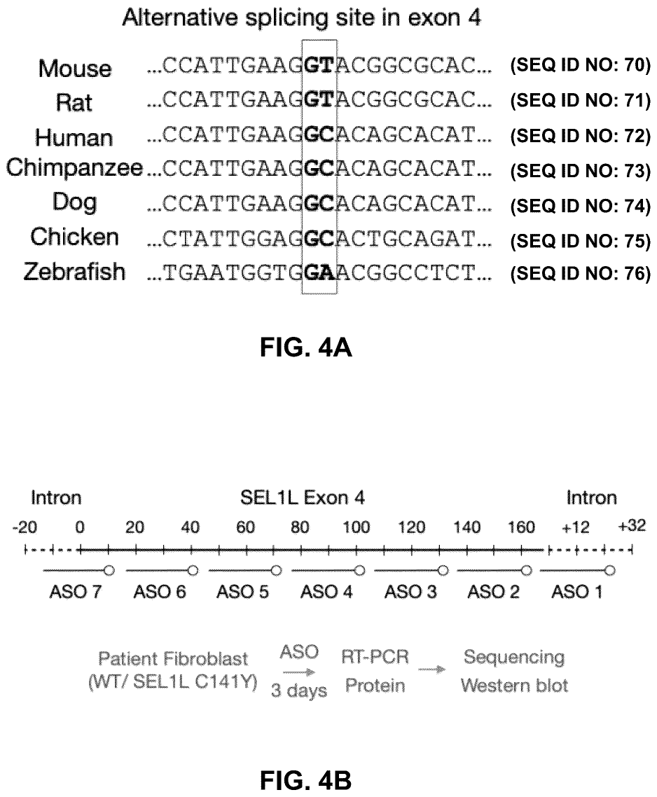

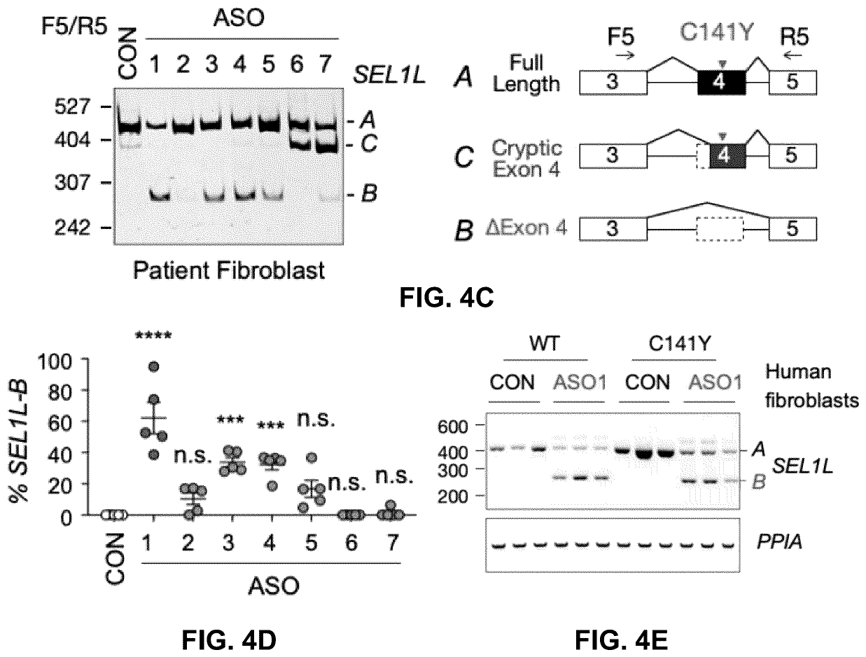

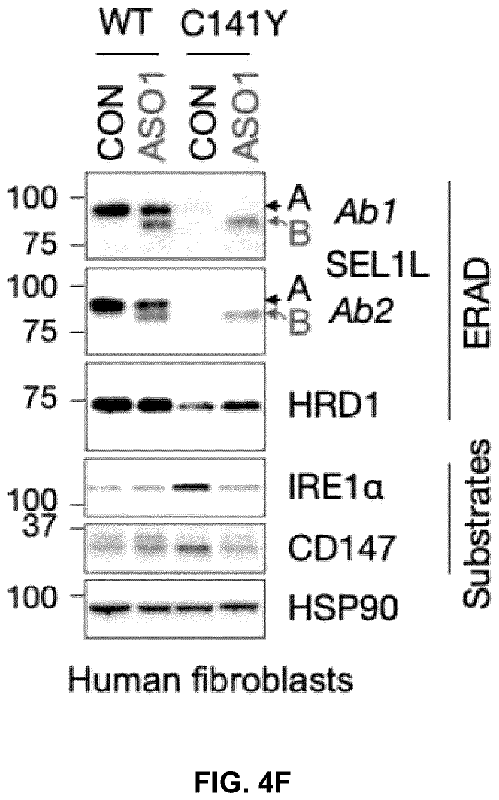

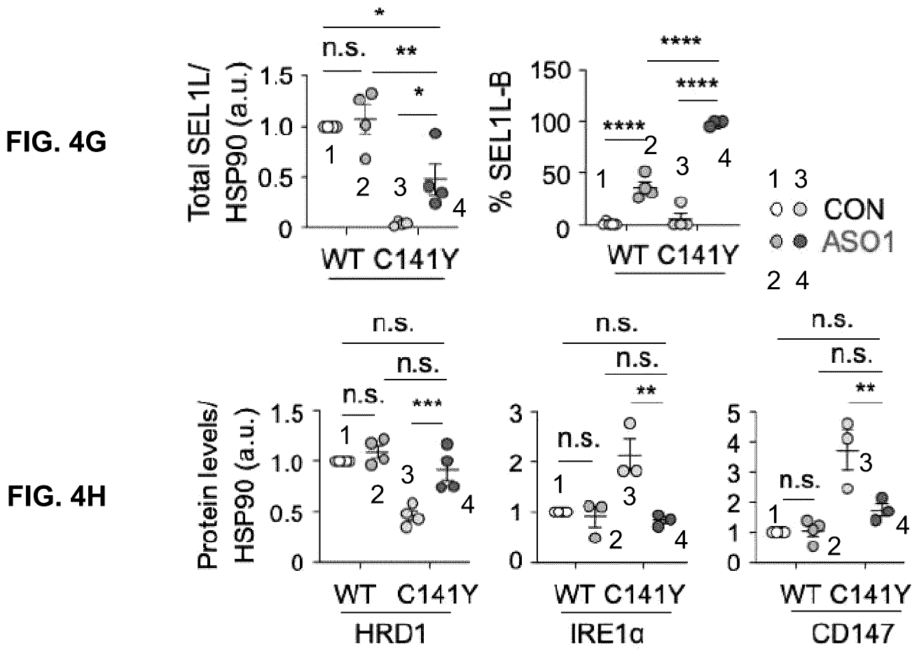

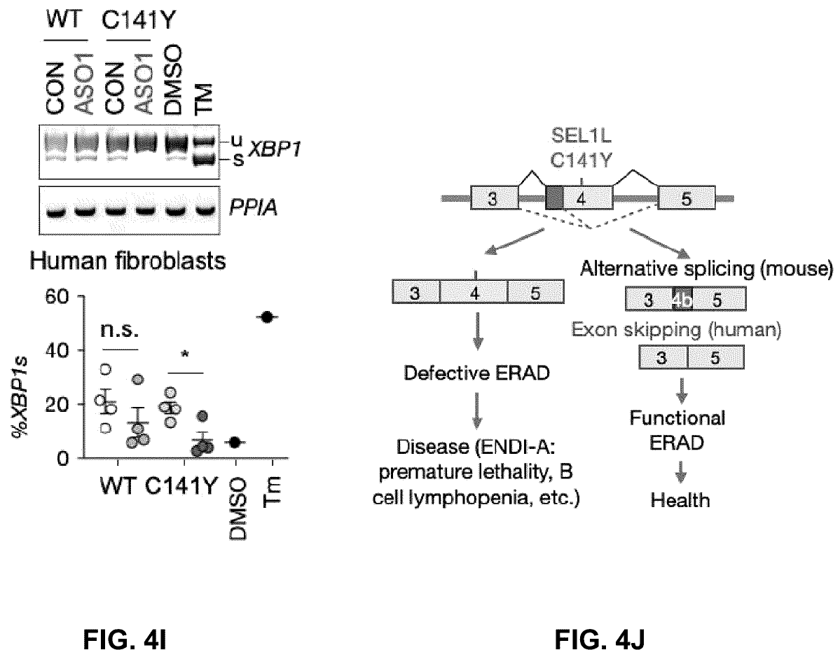

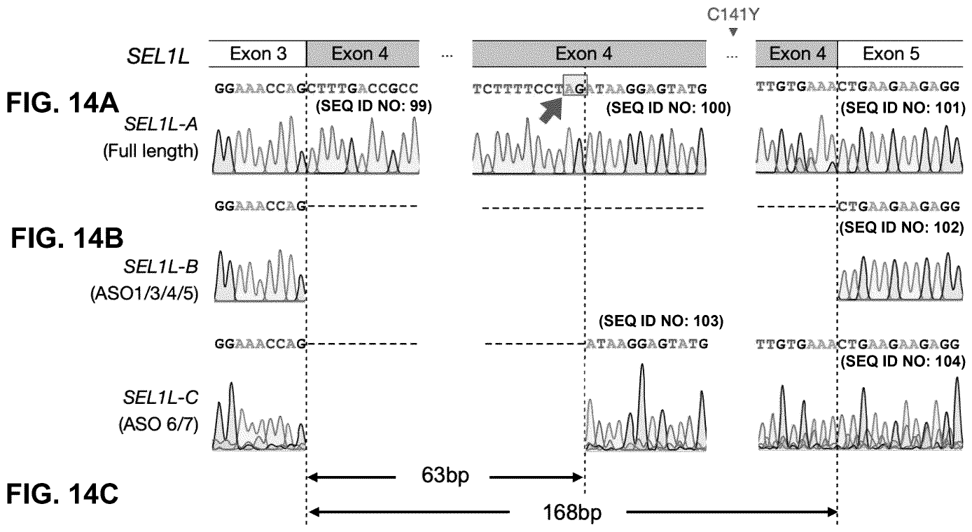

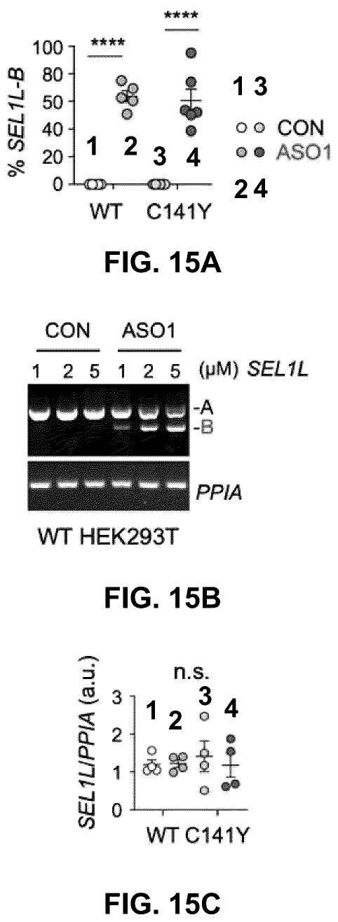

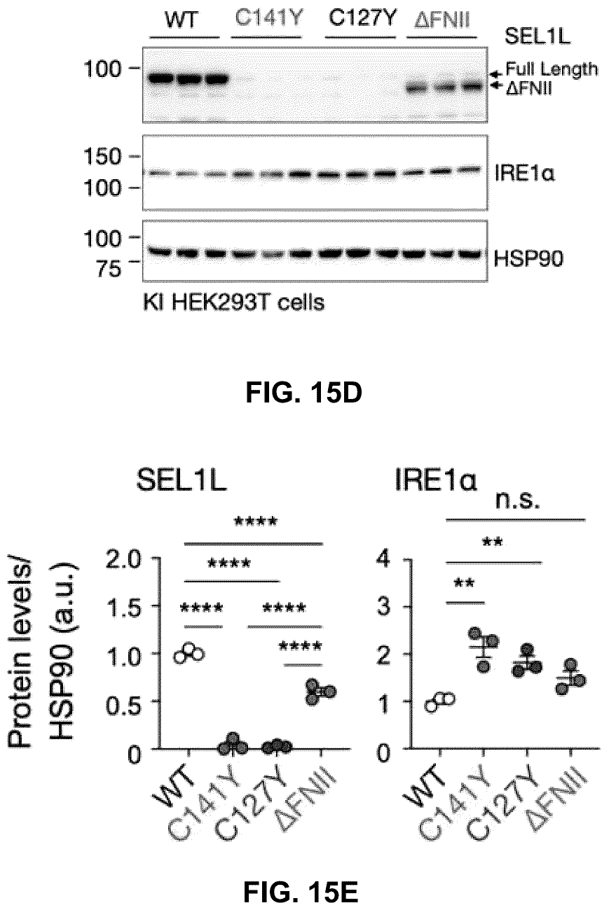

Prompted by serendipitous observations in mice, it was next explored whether an exon-skipping strategy could similarly be applied to human cells harboring the C141Y mutation. Interestingly, sequence alignment revealed that the internal “GU” splice donor site found in murine exon 4 is not conserved in humans or other species (FIG. 4A), consistent with the exclusive expression of the full-length SEL1L-A isoform in human tissues (FIG. 1E). To test the feasibility of exon skipping in this context, fibroblasts from a SEL1L C141Y patient were treated with a panel of 25-nucleotide-long ASOs targeting exon 4 and adjacent intronic sequences. RT-PCR using F5R5 flanking primer pair (FIG. 4C), followed by Sanger sequencing, revealed the emergence of two alternative splice variants in addition to the full-length isoform A: isoform B, which lacked the entire exon 4 and was induced by ASO1 and ASO3-5; and isoform C detected with ASO6-7, which lacked the first 63 bp of exon 4, likely due to the disruption of the intron 3 splicing acceptor and activation of a cryptic splice site within exon 4 (FIG. 4C to FIG. 4D and FIG. 14A-14C). Among all the tested ASOs, ASO1—targeting the exon 4-intron 4 junction—was the most effective, inducing exon 4 skipping in approximately 60% of transcripts in both WT and patient fibroblasts (FIG. 4C to FIG. 4E and FIG. 15A). Similar dose-dependent exon-skipping results were observed in WT HEK293T cells (FIG. 15B).

ASO1 treatment significantly increased expression of the truncated SEL1L-B protein in WT fibroblasts, accounting for ˜40% of total SEL1L protein, and rescued SEL1L expression in C141Y patient fibroblasts (FIG. 4F and quantitated in FIG. 4G). Neither SEL1L C141Y mutation nor ASO treatment altered SEL1L mRNA levels (FIG. 15C), suggesting a post-transcriptional effect. Consistent with the role of SEL1L in stabilizing HRD1 (29), ASO1 treatment restored HRD1 protein levels and rescued ERAD function, as evidenced by decreased accumulation of known ERAD substrates including IRE1α (24) and CD147 (30) (FIG. 4F and quantitated in FIG. 4H), and reduced XBP1 mRNA splicing (FIG. 4I).

To further validate these findings, CRISPR/Cas9 was employed to engineered KI HEK293T cells. While introduction of the C141Y or C127Y mutation abolished SEL1L protein expression, complete deletion of the FNII domain produced a truncated SEL1L protein (˜5 kDa smaller than WT) that preserved normal ERAD function, as reflected by unchanged IRE1a levels (18) (FIG. 15D TO 15E). These results indicate that although disulfide bonds within the FNII domain are critical for SEL1L function, the domain itself is dispensable for ERAD function. Taken together, these findings demonstrate that ASO-mediated exon skipping restores functional SEL1L and rescues ERAD defects in patient-derived fibroblasts, highlighting its therapeutic potential for patients with SEL1L defects.

### Discussion

The SEL1L-HRD1 ERAD pathway is a highly conserved protein quality control mechanism that plays a central role in maintaining ER homeostasis (14). Biallelic mutations in SEL1L and HRD1 were recently identified in patients with ENDI and ENDI-A syndromes (17, 18), implicating ERAD dysfunction in the pathogenesis of these rare but severe genetic disorders. Using a KI mouse model, pathogenicity of the SEL1L C141Y variant located in the fibronectin type II (FNII) domain was established. Moreover, although this residue is essential for SEL1L protein stability, it was found that the FNII domain itself is dispensable for SEL1L function and ERAD activity. This unexpected finding, rooted in the phenotypic discrepancies among three independently generated KI mouse lines carrying the same mutation, led to the discovery of naturally occurring alternative splicing as a mechanism of functional rescue (FIG. 4J). Thus, this example not only defines the molecular pathology of the C141Y mutation but also highlights splicing modulation as a promising therapeutic strategy for correcting ERAD defects.

The SEL1L FNII domain is conserved across vertebrates and is also found in proteins such as fibronectin and tissue-type plasminogen activator/coagulation factor XII (18, 22), but it is absent in yeast SEL1L counterpart Hrd3. This pattern suggests that the domain may have been evolutionarily acquired during vertebrate evolution through exon shuffling (31). In rodent SEL1L, this domain is encoded by exon 4, which undergoes alternative splicing to generate isoform diversity—a feature not observed in humans. This rodent-specific splicing event may provide an added layer of control over SEL1L function. Although the precise role of the FNII domain remains incompletely define, the data herein indicate that SEL1L-B, which lacks this domain, is less stable than the full-length protein, suggesting a role in stabilizing SEL1L. In addition, a recent study implicated SEL1L in collagen turnover and proposed that its FNII domain may contribute to collagen binding (32). While it is speculated that the FNII domain of SEL1L may be involved in substrate recruitment, further studies will be needed to delineate its precise function and broader biological relevance.

Although overexpression of SEL1L or HRD1 has not been directly linked to toxicity in vivo, excessive ERAD activity has been associated with cancer progression (33-35), dampening enthusiasm for long-term gene replacement approaches. Similar challenges exist in other dosage-sensitive disorders; for example, loss-of-function mutations in MECP2 cause Rett syndrome, whereas duplications of the same gene result in a separate neurodevelopmental disorder (36, 37). In this context, this example shows that exon 4 skipping whether naturally occurring in mice or induced by ASOs in human cells—can restore ERAD function (FIG. 4J). By generating a truncated yet functional SEL1L protein that bypasses the fatal mutation, ASO-mediated exon skipping overcomes the instability of mutant SEL1L and avoids potential toxicity associated with gene overexpression.

The clinical success of ASO therapies, such as eteplirsen for Duchenne muscular dystrophy and approved treatments for spinal muscular atrophy and amyotrophic lateral sclerosis, underscores their utility in correcting genetic defects by restoring partially functional proteins with high specificity and minimal off-target effects (21, 38, 39). Intrathecal delivery of ASOs has shown promise in treating rare neurodevelopmental disorders, with substantial improvements in seizures and cognitive outcomes when administered early (40-43). To date, ASOs have not been applied to diseases involving ERAD dysfunction. These findings establish a proof-of-concept for this approach, demonstrating that targeted exon skipping can restore proteostasis in the setting of a pathogenic SEL1L mutation. Given the essential role of SEL1L-HRD1 ERAD in cellular homeostasis (3, 4, 14, 16), this strategy may hold promise not only for SEL1L-related disorders but also for a broader range of protein misfolding diseases.

### Example 2: Materials and Methods Relating to Example 1

### Study Design

This study investigates the pathogenicity of the SEL1L C141Y mutation and evaluates the therapeutic potential of alternative splicing in vivo and via antisense oligonucleotide (ASO)-mediated exon skipping in patient-derived fibroblasts. All animal procedures were approved by the Institutional Animal Care and Use Committee of the University of Michigan Medical School (PRO0008989 and PRO00010658), and University of Virginia Medical School (Protocol No. 4459) and in accordance with the National Institutes of Health (NIH) guidelines. Sample sizes were determined on the basis of prior literature and power calculations to ensure statistically significant results while minimizing the number of animals used in accordance with ethical guidelines. Blinding was applied during running flow cytometry, complete blood count, and histological analysis. Experiments were replicated at least 3 times to ensure reproducibility, and sample sizes for each study are reported in the figure legends. Data inclusion criteria required samples to meet experimental conditions without visible contamination or procedural errors, and exclusion criteria involved procedural inconsistencies, or abnormal physiological conditions in animals (e.g. severe dystocia in breeder female for pup survival experiment). Outliers were assessed both visually using Q-Q plots and statistically using the Shapiro-Wilk or Kolmogorov-Smirnov tests (for sample sizes greater than three), with a significance threshold of α=0.05. Outliers linked to experimental or methodological errors were excluded, while those reflecting biological variation were retained. For expanded Materials and Methods used in this article, please see the Supplementary Materials.

### Mice

The SEL1L^{C141Y} knock-in (KI) mice (human SEL1L p.Cys141 is equivalent to mouse SEL1L p.Cys137) were generated at the University of Michigan Molecular Genetics Core using the CRISPR-Cas9 technology. The two single guide RNAs (sgRNAs) were designed by using computer algorithm (http://crispor.tefor.net), targeting mouse genome SEL1L exon 4 where the mutation is located: sgRNA1: 5′-ATGAGTGCACCTCAGACGGG-3′ (SEQ ID NO: 8); sgRNA2: 5′-AAGTGCGTATTGTTCAAGTG-3′ (SEQ ID NO: 9). The sgRNAs were synthesized using the Synthego sgRNA synthesis Kit per manufacturer's protocol, tested and confirmed in fertilized eggs. A donor DNA carrying mouse Sel1L cDNA 410G>A mutation and additional silent mutations was designed to mediate homology-directed repair (HDR): 5′-CACGGGGAGCCCTGCCACTTCCCTTTCCTTTTCCTGGATAAGGAGTATGATGAGTACAC ATCCGATGGAAGAGAAGATGGCAGACTGTGGTGTGCCACAACCTATGACTACAAGACA GATGAGAAGTGGGGCTTCTGCGAAAGTGCGTATTGTTCAAGTGCACGCCCTGTGCTTT AGGGCAGCATTTGGAAGGCATTTTC-3′ (SEQ ID NO: 10). The donor DNA was synthesized as Ultramer dsDNA by Integrated DNA Technologies, Inc. A mixture of Cas9 protein (Sigma), sgRNAs, and donor DNA was microinjected into fertilized mouse eggs on the C57BL6/J background. The injected zygotes were then transferred into pseudopregnant females. The existence of desired mutations was further confirmed by Sanger sequencing in three independent founders. The founders were then bred separately to WT C57BL6/J mice to obtain F1 heterozygous SEL1L^{C141Y} heterozygous mice. F1 heterozygous SEL1L^{C141Y} mice were inter-crossed to generate homozygous SEL1L^{C141Y} KI mice and its WT and heterozygous littermates. For survival curve, pregnant female and the litter size were checked every 4 hours. Pup tails were collected for genotyping. Age- and gender-matched littermates were maintained in a temperature-controlled room on a 12-h light/dark cycle and used in all studies. For in vivo ER stress induction, 12 weeks old male WT mice were used for UPR activation by DMSO or tunicamycin treatment at 1 mg/kg for 4 hours via intraperitoneal injection.

### Statistical Analysis

Statistics tests were performed in GraphPad Prism version 10.0 (GraphPad Software). Unless indicated otherwise, values are presented as mean±standard error of the mean (SEM). All experiments have been repeated at least three times and/or performed with multiple independent biological samples from which representative data are shown. Statistical differences between the groups were compared using the unpaired two-tailed Student's t-test for two groups, one-way ANOVA followed by Tukey's post hoc test, two-way ANOVA followed by Tukey's multiple-comparisons test for multiple groups, Chi-square test for contingency table, Mantel-Cox test for survival curve. P<0.05 was considered statistically significant.

### Example 3: Additional Details of Examples 1 and 2

### Human Liver Tissue

Deidentified frozen normal human liver tissue was acquired through Biorepository and Tissue Research Facility Core at the University of Virginia.

### Genotyping, PCR and Sequencing

Mice were routinely genotyped using PCR of genomic DNA samples obtained from tails or ears with the following primer pairs:

Sel1L^{C141Y} Allele for SEL1L^{C141Y} KI Mice (FIG. 5B to FIG. 5C):

F:
(SEQ ID NO: 11)
5′-AGTACACATCCGATGGAAGAGAAG-3′;
R:
(SEQ ID NO: 12)
5′-GAAAATGCCTTCCAAATGCTGC-3′;

### Sel1L Wildtype Allele (FIG. 5B to FIG. 5C):

F:
(SEQ ID NO: 13)
5′-AGTGCACCTCAGACGGGAGGG-3′;
R:
(SEQ ID NO: 14)
5′-GAAAATGCCTTCCAAATGCTGC-3′;

Mouse tail or ear genomic DNA sample or cell genomic DNA was used for sanger sequencing analysis by using following primers for PCR and sequencing:

Sequencing for mSel1L Genomic DNA Exon 4 and Intron 4 (FIG. 2A):

F:
(SEQ ID NO: 15)
5′-CTTAAGAACTCAAAGTCTACACTAAGTCT-3′;
R:
(SEQ ID NO: 16)
5′-CAGCTTGCCTCAAGGGTTTACAGAA-3′;

### Histology and Immunofluorescence

Adult mice were anesthetized and perfused with 20 ml of PBS followed by 40 ml of 4% paraformaldehyde in 0.1 M PBS pH 7.4 for fixation. Tissues were dissected out and fixed overnight in 4% paraformaldehyde in PBS at 4° C. For hematoxylin and eosin (H&E) staining, samples were dehydrated, embedded in paraffin, and stained at the Research Histology Core at the University of Virginia School of Medicine. Images were captured using Aperio ScanScope CS System and Aperio ImageScope software.

For immunofluorescence staining, postnatal P0 pups were anesthetized with hypothermia. Then, pup spleen and brain were dissected followed by overnight fixation in 5 mL 4% paraformaldehyde in 0.1 M PBS pH 7.4 under 4° C. The tissues were then transferred to 30% sucrose in PBS solution for overnight incubation under 4° C. and then embed in OCT for cryosection. The tissues were sectioned at 8-15 μM. The samples were blocked with 5% normal donkey serum, 0.3% Tween-20 PBS solution for 1 hour and then incubated with primary antibody diluted in 5% normal donkey serum, 0.3% Tween-20 PBS solution for overnight at 4° C.: anti-CD19 (Cell Signaling Technology, #90176S, 1:100); anti-SATB2 (Abcam, ab51502, 1:20); anti-CTIP2 (Abcam, ab18465, 1:100). The samples were washed with PBS for 5 minutes×3 times at room temperature, and then incubated with secondary antibody for 1 hour at room temperature: Alexa Fluor 555-conjugated donkey anti-rabbit IgG (Jackson ImmunoResearch, 711-565-152, 1:500), Alexa Fluor 488-conjugated goat anti-rat IgG (Invitrogen, A-11006, 1:500), Alexa Fluor 555-conjugated donkey anti-mouse IgG (Invitrogen, A32773, 1:500). After 5 minutes×3 times washing with PBS at room temperature, the slides were mounted in ProLong™ Diamond Antifade Mountant with DAPI (Fisher Scientific P36971). The samples were imaged by Leica DMi8 THUNDER Imager.

### Plasmids

The following plasmids were used in the study: pcDNA5-GFP-IL7R-XbaI mutated minigene construct was a kind gift from Dr. Muge N Kuyumcu-Martinez Lab (1). Then, pcDNA5-GFP-SEL1Lexon4 were generated by GenScript Biotech, by replacing IL7 exon in the pcDNA5-GFP-IL7-XbaI mutated minigene construct with mouse SEL1L exon 4 and the proximal introns (348 bp upstream and 462 bp downstream). Alternative splicing donor site mutation and exon 4 splicing donor site mutation were generated using the above mentioned pcDNA5-GFP-SEL1Lexon4 minigene construct. All plasmids were validated by DNA sequencing. The primers used for mutagenesis in FIG. 2D and FIG. 2G are:

### Mutagenesis Primers for Alternative Splicing Donor Site (Mutation 1):

F
(SEQ ID NO: 31)
5′-CCATTGAAGGCACGGCGCACGGGGA-3′;
R
(SEQ ID NO: 32)
5′-GCGCCGTGCCTTCAATGGCAGTCAAGA-3′;

### Mutagenesis Primers for Exon 4 Canonical Splicing Donor Site Mutation (Mutation 2):

F
(SEQ ID NO: 33)
5′-CTTCTGCGAAAGGCGTATTGTTCAAGTGGGG-3′;
R
(SEQ ID NO: 34)
5′-AACAATACGCCTTTCGCAGAAGCCCCACTTCT-3′;

### Mutagenesis Primers for Exon 4 C137Y Mutation (Mutation 3):

F
(SEQ ID NO: 35)
5′-GTATGATGAGTACACCTCAGACG-3′
R
(SEQ ID NO: 36)
5′-CGTCTGAGGTGTACTCATCATAC-3′

### Mutagenesis Primers for Exon 4 Synonymous Mutations (Mutation 4):

F
(SEQ ID NO: 106)
5′-GATGAGTGCACATCCGATGGAAGAGAAGATGGCAGACTGTGG-3′;
R
(SEQ ID NO: 107)
5′-GCCATCTTCTCTTCCATCGGATGTGCACTCATCATACTCC-3′.

### RNA Preparation and RT-PCR

Total RNA was extracted from cell or tissues using TRI Reagent and BCP phase separation reagent (Molecular Research Center, TR 118). For RT-PCR analysis the following primer sequences were used:

mSEL1L Full Length (F1/R1 Used for FIG. 1E)

F:
(SEQ ID NO: 37)
5′-ATGCAGGTCCGCGTCAGGCTGTCGTTGCTGCT-3′;
R:
(SEQ ID NO: 38)
5′-CTACTGTGGTGGCTGCTGCTCTGG-3′;

hSEL1L Full Length (F1′/R1′ Used for FIG. 1E)

F:
(SEQ ID NO: 39)
5′-GCGGCTAGCATGCGGGTCCGGATAGGGCT-3;
R:
(SEQ ID NO: 40)
5′-GCGAAGCTTTTACTGTGGTGGCTGCTGCTCTG-3′;

mSEL1L Exon4 for Acrylamide Gel (F2/R2 Used for FIG. 1G and FIG. 6A and FIG. 6C)

F:
(SEQ ID NO: 41)
5′-AGCAAGACCTACGAAGAACT-3′;
R:
(SEQ ID NO: 42)
5′-GAAGGTACCGATATGCTTCTCTCT-3′;

mSEL1L Exon4 for Agarose Gel (F3/R3 Used for FIG. 7A TO 7E):

F:
(SEQ ID NO: 43)
5′-ATGCAGGTCCGCGTCAGGCTGTCGTTGCTGCT-3′;
R:
(SEQ ID NO: 44)
5′-CTACTGTGGTGGCTGCTGCTCTGG-3′;

### Minigene Exon4-1 (F4A/R4 Used for FIG. 2E and FIG. 2H):

F:
(SEQ ID NO: 45)
5′-TGGTGAGCAAGGGCGAGG-3′;
R:
(SEQ ID NO: 46)
5′-CGTCCTTGAAGAAGATGGTGCG-3′;

### Minigene Exon4-2 (F4B/R4 Used for FIG. 9B and FIG. 9F):

F:
(SEQ ID NO: 47)
5′-GCGAGTCTTGACTGCCATTGAAG-3′;
R:
(SED ID NO: 48)
5′-CGTCCTTGAAGAAGATGGTGCG-3′;

hSEL1L Exon4 (F5/R5 Used for FIG. 4C, 4E and FIG. 15B):

F:
(SEQ ID NO: 49)
5′-GGGGAAAGTGTCACAGAAGATATCAG-3′;
R:
(SED IQ NO: 50)
5′-GACACTCTCTCCAGGGCTTTG-3′;

mXbp1s:

F:
(SED IQ NO: 51)
5′-ACGAGGTTCCAGAGGTGGAG-3′.
R:
(SED IQ NO: 52)
5′-AAGAGGCAACAGTGTCAGAG-3′;

hXBP1s:

F:
(SED IQ NO: 53)
5′-GAATGAAGTGAGGCCAGTGG-3′;
R:
(SED IQ NO: 54)
5′-ACTGGGTCCTTCTGGGTAGA-3′;

mL32:

F:
(SED IQ NO: 55)
5′-GAGCAACAAGAAAACCAAGCA-3′;
R:
(SED IQ NO: 56)
5′-TGCACACAAGCCATCTACTCA-3′;

hPPIA:

F:
(SED IQ NO: 57)
5′-GGCAAATGCTGGACCCAACACA-3′;
R:
(SED IQ NO: 58)
5′-TGCTGGTCTTGCCATTCCTGGA-3′;

RT-PCR products were analyzed by 0.8%-1.5% agarose electrophoresis or 5% polyacrylamide gel electrophoresis gel using CBS Scientific Adjustable Height Vertical Gel System.

### Western Blot and Antibodies

Mouse tissues or cells were harvested and snap-frozen in liquid nitrogen. The proteins were extracted by sonication in NP-40 lysis buffer (50 mM Tris-HCl at pH7.5, 150 mM NaCl, 1% NP-40, 1 mM EDTA) with protease inhibitor (Sigma), DTT (Sigma, 1 mM) and phosphatase inhibitor cocktail (Sigma). Lysates were incubated on ice for 30 min and centrifuged at 16,000 g for 10 min. Supernatants were collected and analyzed for protein concentration using the Bio-Rad Protein Assay Dye (Bio-Rad). 20-50 μg of protein were denatured at 95° C. for 5 min in 5×SDS sample buffer (250 mM Tris-HCl pH 6.8, 10% sodium dodecyl sulfate, 0.05% bromophenol blue, 50% glycerol, and 1.44 M β-mercaptoethanol). Protein was separated on SDS-PAGE, followed by electrophoretic transfer to PVDF (Fisher Scientific) membrane. The blots were incubated in 2% BSA/Tri-buffered saline tween-20 (TBST) overnight at 4° C. with primary antibodies specifically for: HSP90 (Santa Cruz, #sc-13119, 1:5,000), SEL1L (home-made, 1:10,000) (2), SEL1L (Abcam, ab78298, 1:1,000), HRD1 (Proteintech, #13473-1, 1:2,000), CD147 (Proteintech, #11989-1, 1:3,000), IRE1α (Cell Signaling, #3294, 1:2,000), ERLEC1 (Abcam, #ab181166, 1:5,000), BiP/GRP94 (Abcam, #ab21685, 1:5,000), PDI (Enzo, #ADI-SPA-890, 1:5,000), PERK (Cell Signaling, #3192, 1:5000). Membranes were washed with TBST and incubated with secondary antibodies, either HRP conjugated (Bio-Rad, 1:10,000), anti-Rabbit IgG TrueBlot HRP (Rockland, #18-8816-33, 1:500) or anti-Mouse IgG TrueBlot-HRP (Rockland, 18-8817-31, 1:500) at room temperature for 1 h for ECL chemiluminescence detection system (Bio-Rad) development. Phos-tag-based western blot analysis was performed as previously described (3). Band intensity was determined using ImageJ FIJI software.

### Antisense Oligonucleotide (ASO) Design and Treatment

Morpholino ASO were either designed by Morpholino Gene Tool, LLC or by the authors, and synthesized by Morpholino Gene Tool, LLC. Standard Control Oligo (CCTCTTACCTCAGTTACAATTTATA; SEQ ID NO: 105) were purchased from Morpholino Gene Tool, LLC. Morpholino ASO were dissolved in the original vial with sterilized H₂O at 1 mM. The human fibroblasts or HEK293T cells were cultured at 80-100% confluency and treated with 10 μM Morpholino oligo and 6 μM/mL Endo-Porter (Morpholino Gene Tool, LLC, OT-EP-PEG-1) in 10% serum DMEM medium. The cells were collected after 1 or 3 days for RNA or protein analysis, respectively.

### The ASO Sequences are:

ASO1:
(SEQ ID NO: 1)
TGCCTCCTACTGAGCAATACTTACT
ASO2:
(SEQ ID NO: 2)
AAAAGCCCCACTTTTCATCTGCTTT
ASO3:
(SEQ ID NO: 3)
CATAGGTTGTAGCACACCACAGTCT
ASO4:
(SEQ ID NO: 4)
CTTCCCTCCCATCTGATGTATATTC
ASO5:
(SEQ ID NO: 5)
ACTCCTTATCTAGGAAAAGAAAAGG
ASO6:
(SEQ ID NO: 6)
GGCAGGGCTCCCCATGTGCTGTGCC
ASO7:
(SEQ ID NO: 7)
GGCGGTCAAAGCTGGAATGACAAGA

### Complete Blood Count (CBC), Flow Cytometry, Intracellular Staining, and Antibodies

Peripheral blood was collected from surviving KI mice via facial vein into EDTA-coated tubes and complete blood count was analyzed by University of Virginia Center for Comparative Medicine (CCM) Core. Flow cytometric analysis of peripheral blood mononuclear cells (PBMCs) was performed as we described previously (4, 5). In brief, PBMCs were isolated from blood treated with anti-coagulation reagent and red blood cell lysis buffer for 5 minutes at room temperature. The PBMCs were then stained with ZombieNIR Fixable Viability dye (BioLegend 423106) diluted as 1:400 in PBS-Ca²⁺ solution for 10 minutes at room temperature. CD16/CD32 Rat anti-Mouse antibody was then added as 1:100 to PBMCs for blocking at 4° C. for 5 minutes. The PBMCs were then incubated with 1:100 fluorochrome-conjugated antibody against anti-CD45 (30-F11), anti-CD45R/B220 (RA3-6B2), Anti-CD19 (6D5) (BioLegend or eBioscience) at 4° C. for 30 minutes, followed by 2 washes with Flow cytometry (FACS) buffer (5% serum PBS-Ca solution) under 4° C. The samples were then fixed with 2% paraformaldehyde under room temperature for 20 minutes, followed by 2 washes. The samples were stored at 4° C. overnight and analyzed on Cytek Aurora Northern Lights Spectral Flow Cytometer at the University of Virginia Flow Cytometry Core. The .fcs file were analyzed on FCS Express 7 Research.

### REFERENCES FOR THE PRESENT EXAMPLE

1. Wang H H, Biunno I, Sun S, and Qi L. SEL1L-HRD1-mediated ERAD in mammals. Nat Cell Biol. 2025; 27(7):1063-73.
2. Bhattacharya A, and Qi L. ER-associated degradation in health and disease—from substrate to organism. J Cell Sci. 2019; 132(23):jcs232850.
3. Wu S A, Li Z J, and Qi L. Endoplasmic reticulum (ER) protein degradation by ER-associated degradation and ER-phagy. Trends Cell Biol. 2025; 35(7):576-91.
4. Wang H H, Lin L L, Li Z J, Wei X, Askander O, Cappuccio G, et al. Hypomorphic variants of SEL1L-HRD1 ER-associated degradation are associated with neurodevelopmental disorders. J Clin Invest. 2024; 134(2):e170054.
5. Weis D, Lin L L, Wang H H, Li Z J, Kusikova K, Ciznar P, et al. Biallelic Cys141Tyr variant of SEL1L is associated with neurodevelopmental disorders, agammaglobulinemia, and premature death. J Clin Invest. 2024; 134(2):e170882.
6. Lin L L, Wang H H, Pederson B, Wei X, Torres M, Lu Y, et al. SEL1L-HRD1 interaction is required to form a functional HRD1 ERAD complex. Nature communications. 2024; 15(1):1440.

### REFERENCES RELATED TO THE PRESENT DISCLOSURE

1. R. Y. Hampton, R. G. Gardner, J. Rine, Role of 26S proteasome and HRD genes in the degradation of 3-hydroxy-3-methylglutaryl-CoA reductase, an integral endoplasmic reticulum membrane protein. Mol Biol Cell 7, 2029-2044 (1996).
2. C. L. Ward, S. Omura, R. R. Kopito, Degradation of CFTR by the ubiquitin-proteasome pathway. Cell 83, 121-127 (1995).
3. Z. Sun, J. L. Brodsky, Protein quality control in the secretory pathway. J Cell Biol 218, 3171-3187 (2019).
4. J. C. Christianson, E. Jarosch, T. Sommer, Mechanisms of substrate processing during ER-associated protein degradation. Nat Rev Mol Cell Biol 24, 777-796 (2023).
5. J. Bordallo, R. K. Plemper, A. Finger, D. H. Wolf, Der3p/Hrd1p is required for endoplasmic reticulum-associated degradation of misfolded lumenal and integral membrane proteins. Mol Biol Cell 9, 209-222 (1998).
6. A. T. van der Goot, M. M. P. Pearce, D. E. Leto, T. A. Shaler, R. R. Kopito, Redundant and Antagonistic Roles of XTP3B and OS9 in Decoding Glycan and Non-glycan Degrons in ER-Associated Degradation. Mol Cell 70, 516-530 e516 (2018).
7. N. Hosokawa et al., Human XTP3-B forms an endoplasmic reticulum quality control scaffold with the HRD1-SEL1L ubiquitin ligase complex and BiP. The Journal of biological chemistry 283, 20914-20924 (2008).
8. J. C. Christianson, T. A. Shaler, R. E. Tyler, R. R. Kopito, OS-9 and GRP94 deliver mutant alpha1-antitrypsin to the Hrd1-SEL1L ubiquitin ligase complex for ERAD. Nat Cell Biol 10, 272-282 (2008).
9. B. N. Lilley, H. L. Ploegh, A membrane protein required for dislocation of misfolded proteins from the ER. Nature 429, 834-840 (2004).
10. Y. Oda et al., Derlin-2 and Derlin-3 are regulated by the mammalian unfolded protein response and are required for ER-associated degradation. J Cell Biol 172, 383-393 (2006).
11. L. L. Lin et al., SEL1L-HRD1 interaction is required to form a functional HRD1 ERAD complex. Nat Commun 15, 1440 (2024).
12. B. Mueller, E. J. Klemm, E. Spooner, J. H. Claessen, H. L. Ploegh, SEL1L nucleates a protein complex required for dislocation of misfolded glycoproteins. Proc Natl Acad Sci USA 105, 12325-12330 (2008).
13. R. G. Gardner et al., Endoplasmic reticulum degradation requires lumen to cytosol 50 signaling. Transmembrane control of Hrd1p by Hrd3p. J Cell Biol 151, 69-82 (2000).
14. J. Hwang, L. Qi, Quality Control in the Endoplasmic Reticulum: Crosstalk between ERAD and UPR pathways. Trends Biochem Sci 43, 593-605 (2018).
15. A. Bhattacharya, L. Qi, ER-associated degradation in health and disease—from substrate to organism. J Cell Sci 132, jcs232850 (2019).
16. S. A. Wu, Z. J. Li, L. Qi, Endoplasmic reticulum (ER) protein degradation by ER-associated degradation and ER-phagy. Trends Cell Biol, S0962-8924(0925)00002-00009 (2025).
17. H. H. Wang et al., Hypomorphic variants of SEL1L-HRD1 ER-associated degradation are associated with neurodevelopmental disorders. J Clin Invest 134, (2024).
18. D. Weis et al., Biallelic Cys141Tyr variant of SEL1L is associated with neurodevelopmental disorders, agammaglobulinemia, and premature death. J Clin Invest 134, (2024).
19. M. C. Lauffer, W. van Roon-Mom, A. Aartsma-Rus, N. Collaborative, Possibilities and limitations of antisense oligonucleotide therapies for the treatment of monogenic disorders. Commun Med (Lond) 4, 6 (2024).
20. Y. Zhang, J. Qian, C. Gu, Y. Yang, Alternative splicing and cancer: a systematic review. Signal Transduct Target Ther 6, 78 (2021).
21. S. T. Crooke, B. F. Baker, R. M. Crooke, X. H. Liang, Antisense technology: an overview and prospectus. Nat Rev Drug Discov 20, 427-453 (2021).
22. Y. Harada et al., Complete cDNA sequence and genomic organization of a human pancreas-specific gene homologous to Caenorhabditis elegans sel-1. J Hum Genet 44, 330-336 (1999).
23. A. R. Pickford, S. P. Smith, D. Staunton, J. Boyd, I. D. Campbell, The hairpin structure of the (6)F1(1)F2(2)F2 fragment from human fibronectin enhances gelatin binding. EMBO J 20, 1519-1529 (2001).
24. S. Sun et al., IRE1α is an endogenous substrate of endoplasmic-reticulum-associated degradation. Nat Cell Biol 17, 1546-1555 (2015).
25. K. Belanger et al., CELF1 contributes to aberrant alternative splicing patterns in the type 1 diabetic heart. Biochem Biophys Res Commun 503, 3205-3211 (2018).
26. Y. Ji et al., The Sel1L-Hrd1 Endoplasmic Reticulum-Associated Degradation Complex Manages a Key Checkpoint in B Cell Development. Cell Rep 16, 2630-2640 (2016).
27. N. Xia et al., B Lymphocyte-Deficiency in Mice Causes Vascular Dysfunction by Inducing Neutrophilia. Biomedicines 9, (2021).
28. J. Stiles, T. L. Jernigan, The basics of brain development. Neuropsychol Rev 20, 327-348 (2010).
29. S. Sun et al., Sel1L is indispensable for mammalian endoplasmic reticulum-associated degradation, endoplasmic reticulum homeostasis, and survival. Proceedings of the National Academy of Sciences of the United States of America 111, E582-591 (2014).
30. R. E. Tyler et al., Unassembled CD147 is an endogenous endoplasmic reticulum-associated degradation substrate. Mol Biol Cell 23, 4668-4678 (2012).
31. R. S. Patel, E. Odermatt, J. E. Schwarzbauer, R. O. Hynes, Organization of the fibronectin gene provides evidence for exon shuffling during evolution. EMBO J 6, 2565-2572 (1987).
32. M. J. Podolsky et al., Genome-wide screens identify SEL1L as an intracellular rheostat controlling collagen turnover. Nat Commun 15, 1531 (2024).
33. I. Biunno et al., SEL1L a multifaceted protein playing a role in tumor progression. J Cell Physiol 208, 23-38 (2006).
34. H. Kim, A. Bhattacharya, L. Qi, Endoplasmic reticulum quality control in cancer: Friend or foe. Semin Cancer Biol 33, 25-33 (2015).
35. N. Karamali, S. Ebrahimnezhad, R. Khaleghi Moghadam, N. Daneshfar, A. Rezaiemanesh, HRD1 in human malignant neoplasms: Molecular mechanisms and novel therapeutic strategy for cancer. Life Sci 301, 120620 (2022).
36. J. P. K. Ip, N. Mellios, M. Sur, Rett syndrome: insights into genetic, molecular and circuit mechanisms. Nat Rev Neurosci 19, 368-382 (2018).
37. D. Lugtenberg et al., Structural variation in Xq28: MECP2 duplications in 1% of patients with unexplained XLMR and in 2% of male patients with severe encephalopathy. Eur J Hum Genet 17, 444-453 (2009).
38. K. R. Lim, R. Maruyama, T. Yokota, Eteplirsen in the treatment of Duchenne muscular dystrophy. Drug Des Devel Ther 11, 533-545 (2017).
39. Y. J. Kim et al., Exon-skipping antisense oligonucleotides for cystic fibrosis therapy. Proc Natl Acad Sci USA 119, (2022).
40. A. Ziegler et al., Antisense oligonucleotide therapy in an individual with KIF1A-associated neurological disorder. Nat Med 30, 2782-2786 (2024).
41. M. Wagner et al., Antisense oligonucleotide treatment in a preterm infant with early-onset SCN2A developmental and epileptic encephalopathy. Nat Med, (2025).
42. X. Chen et al., Antisense oligonucleotide therapeutic approach for Timothy syndrome. Nature 628, 818-825 (2024).
43. Y. Sztainberg et al., Reversal of phenotypes in MECP2 duplication mice using genetic rescue or antisense oligonucleotides. Nature 528, 123-126 (2015).

### INFORMAL SEQUENCE LISTING

The following provides a brief description of oligonucleotide (DNA/RNA) and peptide sequences referred to in the present disclosure. This list may not be exhaustive and other sequences may be referred to by designations known to those of skill in the art.

| | | SEQ |
|---|---|---|
| | | ID |
| Description | Sequence (5′→3′) | NO: |
| ASO1 | TGCCTCCTACTGAGCAATACTTACT | 1 |
| ASO2 | AAAAGCCCCACTTTTCATCTGCTTT | 2 |
| ASO3 | CATAGGTTGTAGCACACCACAGTCT | 3 |
| ASO4 | CTTCCCTCCCATCTGATGTATATTC | 4 |
| ASO5 | ACTCCTTATCTAGGAAAAGAAAAGG | 5 |
| ASO6 | GGCAGGGCTCCCCATGTGCTGTGCC | 6 |
| ASO7: | GGCGGTCAAAGCTGGAATGACAAGA | 7 |
| GapmerASO1 | TGGAATCTAAGGATTCATCCTGG | 8 |
| GapmerASO2 | ACTCTGCCTGCAGTAGTATGGTC | 9 |
| GapmerASO3 | ATATCTTCTGTGACACTTTCCC | 10 |
| GapmerASO4 | TACTTTCTTTGGCTCTTCATAGT | 11 |
| GapmerASO5 | GCAGGGCTCCCCATGTGCTGTGC | 12 |
| GapmerASO6 | TCATAGGTTGTAGCACACCACAG | 13 |
| GapmerASO7 | GCCGTCTCTTAGCAGCCTCTTCT | 14 |
| GapmerASO8 | TCCCTTGGGAGAGCCTTCCTCA | 15 |
| GapmerASO9 | TAAAGTAGTGGAGAGCTGTCTCA | 16 |
| GapmerASO10 | ACTTGAACTCCTCTCCCATAGAG | 17 |
| GapmerASO11 | TCAGACCAACGGCCTCGTTCACA | 18 |
| GapmerASO12 | CAATGATGGTCATGAGGTAAAGG | 19 |
| Mouse Sel1L cDNA | atgcaggtccgcgtcaggctgtcgttgctgctgctctgcgcggtgctcctgggctc | 20 |
| sequence | ggcagccgcgacctcggatgacaaaactaaccaggatgactccttagattcca | |
| | agagttctttgcccacagatgagtcagtgaaggaccacaccaccacgggcaaa | |
| | gtagttgctggccagatatttgttgattctgaagaagcagaagtggaatcccttc | |
| | ttcaggacgaggaagatagctccaagacccaggaggaagagatcagcttttta | |
| | gaatctccgaatccaagcagcaagacctacgaagaactaaagagagtgcgga | |
| | agccagtcttgactgccattgaaggtacggcgcacggggagccctgccacttcc | |
| | ctttccttttcctggataaggagtatgatgagtgcacctcagacgggagggaag | |
| | atggcagactgtggtgtgccacaacctatgactacaagacagatgagaagtgg | |
| | ggcttctgcgaaactgaagaagatgctgccaaaagacgacagatgcaggaag | |
| | cagagatgatctatcaggccgggatgaagatactgaatggaagcaataggaag | |
| | agccaaaagagagaagcatatcggtaccttcagaaggcagcaggcatgaatc | |
| | acaccaaagccctggagagagtgtcctatgctctcttgtttggtgattacctcac | |
| | acagaatatccaggcagccaaagagatgtttgagaaactgactgaggaagggt | |
| | ctcccaaaggacagactggtcttggctttctctacgcttctgggcttggtgttaatt | |
| | caagtcaggcaaaggctcttgtatattatactttcggagctcttggaggcaacct | |
| | gatagcccatatgattttgggttaccgctactgggctggcatcggagtcctccag | |
| | agttgtgagtcggcactgacccattatcgtcttgttgccaatcatgttgctagtga | |
| | tatctccctaactggaggctctgtagtccagagaatacggctgcccgatgaagtg | |
| | gaaaacccggggatgaacagtgggatgctggaagaagacctgattcagtatta | |
| | ccagttcctagctgagaagggtgacgtccaagcacaggttggcctgggacagct | |
| | gcatctgcatggagggcgtggagtagaacagaatcaccagagagcgtttgact | |
| | acttcaacttagcagcaaatgctggcaattcacatgctatggccttcctgggaaa | |
| | gatgtattctgaaggaagtgacatcgtacctcagagtaatgagacggcacttca | |
| | ctactttaagaaagctgctgacatgggcaaccccgtgggacagagcgggcttgg | |
| | aatggcctacctctacggaagaggcgttcaagttaattatgacctggccctcaa | |
| | gtatttccagaaagctgctgagcaaggctgggtggacgggcagctgcagctcg | |
| | gctctatgtactacaatggcattggagtcaagagagattataagcaggccttga | |
| | agtattttaatctggcttctcaaggaggccatatcttggctttctataacctcgcac | |
| | agatgcacgccagcggcacaggggtgatgcggtcctgtcacactgcagtggag | |
| | ttgtttaagaatgtgtgtgagcgaggtcgctggtcagagagactgatgactgcct | |
| | acaacagctataaggatgaggactacaatgctgcagtggtccagtacctcctgc | |
| | tggctgagcagggctacgaggtggcgcagagcaacgcagccttcatcctcgac | |
| | cagagagaagcaaccattgtaggtgagaatgaaacttaccccagagctttactg | |
| | cattggaacagggccgcctcccaaggttacactgtggctagaattaagcttgga | |
| | gactaccacttctatggctttggcactgatgtggattatgagaccgcatttattca | |
| | ttaccgcctggcttctgagcagcagcacagcgcccaagctatgtttaacctgggc | |
| | tacatgcacgagaagggcctaggcattaaacaggacattcaccttgcaaaacg | |
| | cttttatgacatggcagccgaagctagcccagatgcacaagtacctgtgttcctc | |
| | gcactctgcaaattaggtgtcgtctatttcttacagtacatacgggaagcaaaca | |
| | ttcgagatctattcacacaactggatatggaccagcttttgggacccgagtggga | |
| | cctttacctcatgaccatcattgcactgctcttgggtacagtcatagcttacaggc | |
| | agcgacagcaccaggacataccagttcccaggcccccagggccacggccggct | |
| | cctccccagcaggaaggaccaccagagcagcagccaccacagtag | |
| Human SEL1L cDNA | ATGCGGGTCCGGATAGGGCTGACGCTGCTGCTGTGTGCGGT | 21 |
| sequence | GCTGCTGAGCTTGGCCTCGGCGTCCTCGGATGAAGAAGGCA | |
| | GCCAGGATGAATCCTTAGATTCCAAGACTACTTTGACATCAG | |
| | ATGAGTCAGTAAAGGACCATACTACTGCAGGCAGAGTAGTT | |
| | GCTGGTCAAATATTTCTTGATTCAGAAGAATCTGAATTAGAA | |
| | TCCTCTATTCAAGAAGAGGAAGACAGCCTCAAGAGCCAAGA | |
| | GGGGGAAAGTGTCACAGAAGATATCAGCTTTCTAGAGTCTC | |
| | CAAATCCAGAAAACAAGGACTATGAAGAGCCAAAGAAAGTA | |
| | CGGAAACCAGCTTTGACCGCCATTGAAGGCACAGCACATGG | |
| | GGAGCCCTGCCACTTCCCTTTTCTTTTCCTAGATAAGGAGTAT | |
| | GATGAATGTACATCAGATGGGAGGGAAGATGGCAGACTGT | |
| | GGTGTGCTACAACCTATGACTACAAAGCAGATGAAAAGTGG | |
| | GGCTTTTGTGAAACTGAAGAAGAGGCTGCTAAGAGACGGCA | |
| | GATGCAGGAAGCAGAAATGATGTATCAAACTGGAATGAAAA | |
| | TCCTTAATGGAAGCAATAAGAAAAGCCAAAAAAGAGAAGCA | |
| | TATCGGTATCTCCAAAAGGCAGCAAGCATGAACCATACCAAA | |
| | GCCCTGGAGAGAGTGTCATATGCTCTTTTATTTGGTGATTAC | |
| | TTGCCACAGAATATCCAGGCAGCGAGAGAGATGTTTGAGAA | |
| | GCTGACTGAGGAAGGCTCTCCCAAGGGACAGACTGCTCTTG | |
| | GCTTTCTGTATGCCTCTGGACTTGGTGTTAATTCAAGTCAGG | |
| | CAAAGGCTCTTGTATATTATACATTTGGAGCTCTTGGGGGCA | |
| | ATCTAATAGCCCACATGGTTTTGGGTTACAGATACTGGGCTG | |
| | GCATCGGCGTCCTCCAGAGTTGTGAATCTGCCCTGACTCACT | |
| | ATCGTCTTGTTGCCAATCATGTTGCTAGTGATATCTCGCTAAC | |
| | AGGAGGCTCAGTAGTACAGAGAATACGGCTGCCTGATGAAG | |
| | TGGAAAATCCAGGAATGAACAGTGGAATGCTAGAAGAAGAT | |
| | TTGATTCAATATTACCAGTTCCTAGCTGAAAAAGGTGATGTA | |
| | CAAGCACAGGTTGGTCTTGGACAACTGCACCTGCACGGAGG | |
| | GCGTGGAGTAGAACAGAATCATCAGAGAGCATTTGACTACT | |
| | TCAATTTAGCAGCAAATGCTGGCAATTCACATGCCATGGCCT | |
| | TTTTGGGAAAGATGTATTCGGAAGGAAGTGACATTGTACCTC | |
| | AGAGTAATGAGACAGCTCTCCACTACTTTAAGAAAGCTGCTG | |
| | ACATGGGCAACCCAGTTGGACAGAGTGGGCTTGGAATGGCC | |
| | TACCTCTATGGGAGAGGAGTTCAAGTTAATTATGATCTAGCC | |
| | CTTAAGTATTTCCAGAAAGCTGCTGAACAAGGCTGGGTGGA | |
| | TGGGCAGCTACAGCTTGGTTCCATGTACTATAATGGCATTGG | |
| | AGTCAAGAGAGATTATAAACAGGCCTTGAAGTATTTTAATTT | |
| | AGCTTCTCAGGGAGGCCATATCTTGGCTTTCTATAACCTAGC | |
| | TCAGATGCATGCCAGTGGCACCGGCGTGATGCGATCATGTC | |
| | ACACTGCAGTGGAGTTGTTTAAGAATGTATGTGAACGAGGC | |
| | CGTTGGTCTGAAAGGCTTATGACTGCCTATAACAGCTATAAA | |
| | GATGGCGATTACAATGCTGCAGTGATCCAGTACCTCCTCCTG | |
| | GCTGAACAGGGCTATGAAGTGGCACAAAGCAATGCAGCCTT | |
| | TATTCTTGATCAGAGAGAAGCAAGCATTGTAGGTGAGAATG | |
| | AAACTTATCCCAGAGCTTTGCTACATTGGAACAGGGCCGCCT | |
| | CTCAAGGCTATACTGTGGCTAGAATTAAGCTCGGAGACTACC | |
| | ATTTCTATGGGTTTGGCACCGATGTAGATTATGAAACTGCAT | |
| | TTATTCATTACCGTCTGGCTTCTGAGCAGCAACACAGTGCAC | |
| | AAGCTATGTTTAATCTGGGATATATGCATGAGAAAGGACTG | |
| | GGCATTAAACAGGATATTCACCTTGCGAAACGTTTTTATGAC | |
| | ATGGCAGCTGAAGCCAGCCCAGATGCACAAGTTCCAGTCTTC | |
| | CTAGCCCTCTGCAAATTGGGCGTCGTCTATTTCTTGCAGTACA | |
| | TACGGGAAACAAACATTCGAGATATGTTCACCCAACTTGATA | |
| | TGGACCAGCTTTTGGGACCTGAGTGGGACCTTTACCTCATGA | |
| | CCATCATTGCGCTGCTGTTGGGAACAGTCATAGCTTACAGGC | |
| | AAAGGCAGCACCAAGACATGCCTGCACCCAGGCCTCCAGGG | |
| | CCACGGCCAGCTCCACCCCAGCAGGAGGGGCCACCAGAGCA | |
| | GCAGCCACCACAGTAA | |
| sgRNA 1 | ATGAGTGCACCTCAGACGGG | 22 |
| sgRNA 2 | AAGTGCGTATTGTTCAAGTG | 23 |
| Donor DNA | CACGGGGAGCCCTGCCACTTCCCTTTCCTTTTC | 24 |
| | CTGGATAAGGAGTATGATGAGTACACATCCGAT | |
| | GGAAGAGAAGATGGCAGACTGTGGTGTGCCACA | |
| | ACCTATGACTACAAGACAGATGAGAAGTGGGGC | |
| | TTCTGCGAAAGTGCGTATTGTTCAAGTGCACGCC | |
| | CTGTGCTTTAGGGCAGCATTTGGAAGGCATTTTC | |
| Sel1L^{C141Y} allele for | AGTACACATCCGATGGAAGAGAAG | 25 |
| SEL1L^{C141Y} KI mice | | |
| forward primer | | |
| Sel1L^{C141Y} allele for | GAAAATGCCTTCCAAATGCTGC | 26 |
| SEL1L^{C141Y} KI mice | | |
| reverse primer | | |
| Sel1L wildtype allele | AGTGCACCTCAGACGGGAGGG | 27 |
| forward primer | | |
| Sel1L wildtype allele | GAAAATGCCTTCCAAATGCTGC | 28 |
| reverse primer | | |
| mSel1L genomic DNA | CTTAAGAACTCAAAGTCTACACTAAGTCT | 29 |
| Exon 4 and intron 4 | | |
| sequencing forward | | |
| primer | | |
| mSel1L genomic DNA | CAGCTTGCCTCAAGGGTTTACAGAA | 30 |
| Exon 4 and intron 4 | | |
| sequencing forward | | |
| primer | | |
| Alternative splicing | CCATTGAAGGCACGGCGCACGGGGA | 31 |
| donor site | | |
| mutagenesis primer | | |
| forward | | |
| Alternative splicing | GCGCCGTGCCTTCAATGGCAGTCAAGA | 32 |
| donor site | | |
| mutagenesis primer | | |
| reverse | | |
| Forward mutagenesis | CTTCTGCGAAAGGCGTATTGTTCAAGTGGGG | 33 |
| primer for Exon 4 | | |
| canonical splicing | | |
| donor site mutation | | |
| Reverse mutagenesis | AACAATACGCCTTTCGCAGAAGCCCCACTTCT | 34 |
| primer for Exon 4 | | |
| canonical splicing | | |
| donor site mutation | | |
| Forward mutagenesis | GTATGATGAGTACACCTCAGACG | 35 |
| primers for Exon 4 | | |
| C137Y mutation | | |
| Reverse mutagenesis | CGTCTGAGGTGTACTCATCATAC | 36 |
| primers for Exon 4 | | |
| C137Y mutation | | |
| mSEL1L Full length | ATGCAGGTCCGCGTCAGGCTGTCGTTGCTGCT | 37 |
| forward primer | | |
| mSEL1L Full length | CTACTGTGGTGGCTGCTGCTCTGG | 38 |
| reverse primer | | |
| hSEL1L Full length | GCGGCTAGCATGCGGGTCCGGATAGGGCT | 39 |
| forward primer | | |
| hSEL1L Full length | GCGAAGCTTTTACTGTGGTGGCTGCTGCTCTG | 40 |
| reverse primer | | |
| mSEL1L Exon4 for | AGCAAGACCTACGAAGAACT | 41 |
| acrylamide gel | | |
| forward primer | | |
| mSEL1L Exon4 for | GAAGGTACCGATATGCTTCTCTCT | 42 |
| acrylamide gel | | |
| reverse primer | | |
| mSEL1L Exon4 for | ATGCAGGTCCGCGTCAGGCTGTCGTTGCTGCT | 43 |
| agarose gel forward | | |
| primer | | |
| mSEL1L Exon4 for | CTACTGTGGTGGCTGCTGCTCTGG | 44 |
| agarose gel reverse | | |
| primer | | |
| Minigene Exon4-1 | TGGTGAGCAAGGGCGAGG | 45 |
| forward primer | | |
| Minigene Exon4-1 | CGTCCTTGAAGAAGATGGTGCG | 46 |
| reverse primer | | |
| Minigene Exon4-2 | GCGAGTCTTGACTGCCATTGAAG | 47 |
| forward primer | | |
| Minigene Exon4-2 | CGTCCTTGAAGAAGATGGTGCG | 48 |
| reverse primer | | |
| hSEL1L Exon4 | GGGGAAAGTGTCACAGAAGATATCAG | 49 |
| forward primer | | |
| hSEL1L Exon4 | GACACTCTCTCCAGGGCTTTG | 50 |
| reverse primer | | |
| mXbp1s forward | ACGAGGTTCCAGAGGTGGAG | 51 |
| primer | | |
| mXbp1s reverse | AAGAGGCAACAGTGTCAGAG | 52 |
| primer | | |
| hXBP1s forward | GAATGAAGTGAGGCCAGTGG | 53 |
| primer | | |
| hXBP1s reverse | ACTGGGTCCTTCTGGGTAGA | 54 |
| primer | | |
| mL32 forward primer | GAGCAACAAGAAAACCAAGCA | 55 |
| mL32 reverse primer | TGCACACAAGCCATCTACTCA | 56 |
| hPPIA forward primer | GGCAAATGCTGGACCCAACACA | 57 |
| hPPIA reverse primer | TGCTGGTCTTGCCATTCCTGGA | 58 |
| Portion of WT SEL1L | AGTGCACCTCAGACGGGAGGGAA | 59 |
| Portion of SEL1L from | AGTACACATCCGATGGAAGAGAA | 60 |
| KI Founder Lines | | |
| A/B/C | | |
| Portion of SEL1L-a KI | AGCCAGTCTTGACTGCCATTGAAGGTACGGCGC | 61 |
| | ACGGGGA | |
| Portion of SEL1L-a KI | CGAAACTGAAGAAG | 62 |
| Portion of SEL1L-b KI′ | AGCCAGTCTTGACTGCCATTGAAG | 63 |
| Portion of SEL1L-b KI′ | CTGAAGAAG | 64 |
| Portion of SEL1L WT | GCGAAAGTGCGTATT | 65 |
| Portion of SEL1L KI | GCGAAAGTGCGTATT | 66 |
| Portion of SEL1L KI′ | GCGAAAG-GCGTATT | 67 |
| Portion of SEL1L WT | AGTGCACCTCAGACGGGAGGGAA | 68 |
| Portion of SEL1L KI | AGTACACATCCGATGGAAGAGAA | 69 |
| SEL1L Exon 4 Alt | CCATTGAAGGTACGGCGCAC | 70 |
| Spice Site Mouse | | |
| SEL1L Exon 4 Alt | CCATTGAAGGTACGGCGCAC | 71 |
| Spice Site Rat | | |
| SEL1L Exon 4 Alt | CCATTGAAGGCACAGCACAT | 72 |
| Spice Site Human | | |
| SEL1L Exon 4 Alt | CCATTGAAGGCACAGCACAT | 73 |
| Spice Site Chimp. | | |
| SEL1L Exon 4 Alt | CCATTGAAGGCACAGCACAT | 74 |
| Spice Site Dog | | |
| SEL1L Exon 4 Alt | CTATTGGAGGCACTGCAGAT | 75 |
| Spice Site Chicken | | |
| SEL1L Exon 4 Alt | TGAATGGTGGAACGGCCTCT | 76 |
| Spice Site Zebrafish | | |
| Portion of SEL1L FNII | AHGEPCHFPFPLFLDKEYDECTSDGREDGRLWCA | 77 |
| Domain (Human) | TTYDYKADEKWGFCET | |
| Portion of SEL1L FNII | AHGEPCHFPFLFLDKEYDECTSDGREDGRLWCAT | 78 |
| Domain (Chimp.) | TYDYKADEKWGFCET | |
| Portion of SEL1L FNII | AHGEPCHFPOFLFLDKEYDECTSDGREDGRLWCA | 79 |
| Domain (Dog) | TTYDYKADEKWGFCET | |
| Portion of SEL1L FNII | AHGEPCHFPFLFLDKEYDECTSDGREDGRELWCA | 80 |
| Domain (Mouse) | TTYDYKTDEKWGFCET | |
| Portion of SEL1L FNII | AHGEPCHFPFLFLDKEYDECTSDGREDGRLWCAT | 81 |
| Domain (Rat) | TYKYKTDEKWGFCET | |
| Portion of SEL1L FNII | ADGEPCHFPFLFMEKEYAECTADGREDGRLWCAT | 82 |
| Domain (Chicken) | TYDYKKDQKWGFCET | |
| Portion of SEL1L FNII | ASGEPCIFPFLFQGKEYSDCTTEGRGDGRLWCAT | 83 |
| Domain (Zebrafish) | TYDYNNDKRWGFCET | |
| Portion of | TATGATGAGTGCACCTCAGACGGGAGGGAAGAT | 84 |
| Endogenous Mouse | GGC | |
| SEL1L Exon 4 1 | | |
| Portion of | GAAAGTGCGTATTGTTCAAGTGGGGC | 85 |
| Endogenous Mouse | | |
| SEL1L Exon 4 2 | | |
| Portion of | YDECTSDGREDGE | 86 |
| Endogenous Mouse | | |
| SEL1L Exon 4 Protein | | |
| Portion of HDR Donor | TATGATGAGTACACATCCGATGGAAGAGAAGAT | 87 |
| Mouse SEL1L Exon 4 | GGC | |
| 1 | | |
| Portion of HDR Donor | GAAAGTGCGTATTGTTCAAGTGC | 88 |
| Mouse SEL1L Exon 4 | | |
| 2 | | |
| Portion of HDR Donor | YDEYTSDGEDGE | 89 |
| Mouse SEL1L Exon 4 | | |
| Protein | | |
| SEL1L FNII Domain | AAGGAGTATGATGAGTGCACCTCAGACGGGAGG | 90 |
| WT | GAAGATGGC | |
| SEL1L FNII Domain | KEYDECTSDGREDG | 91 |
| WT Amino Acid | | |
| SEL1L FNII Domain | AAGGAGTATGATGAGTECACETCEGAEGGEGEG | 92 |
| Het | AAGATGGC | |
| SEL1L FNII Domain | KEYDE[C/Y]TSDGREDG | 93 |
| Het Amino Acid | | |
| SEL1L FNII Domain | AAGGAGTATGATGAGTACACATCCGATGGAAGA | 94 |
| KI | GAAGATGGC | |
| SEL1L FNII Domain | KEYDEYTSDGREDG | 95 |
| KI Amino Acid | | |
| SEL1L Exon 4a 1 | GGGCGAGTCTTGACTGCCATTGAAGGTACGG | 96 |
| SEL1L Exon 4a 2 | GCGAAAGGCGATGCC | 97 |
| SEL1L Exon 4b | GGGCGAGTCTTGACTGCCATTGAAG | 98 |
| Portion of Full-Length | GGAAACCAGCTTTGACCGCC | 99 |
| SEL1L-A 1 | | |
| Portion of Full-Length | TCTTTTCCTAGATAAGGAGTATG | 100 |
| SEL1L-A 2 | | |
| Portion of Full-Length | TTGTGAAACTGAAGAAGAGG | 101 |
| SEL1L-A 3 | | |
| Portion of Full-Length | CTGAAGAAGAGG | 102 |
| SEL1L-B 1 | | |
| (ASO1/3/4/5) | | |
| Portion of Full-Length | ATAAGGAGTATG | 103 |
| SEL1L-C 1 (ASO 6/7) | | |
| Portion of Full-Length | TTGTGAAACTGAAGAAGAGG | 104 |
| SEL1L-C 2 (ASO 6/7) | | |
| Control Oligo | CCTCTTACCTCAGTTACAATTTATA | 105 |
| Forward mutagenesis | GATGAGTGCACATCCGATGGAAGAGAAGATGGC | 106 |
| primer for Exon 4 | AGACTGTGG | |
| synonymous | | |
| mutations (Mutation 4) | | |
| Forward mutagenesis | GCCATCTTCTCTTCCATCGGATGTGCACTCATCA | 107 |
| primer for Exon 4 | TACTCC | |
| synonymous | | |
| mutations (Mutation 4) | | |

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1. A composition, comprising:
one or more nucleotide-based therapeutics for SEL1L adaptor subunit of SYVN1 ubiquitin ligase-ERAD-associated E3 ubiquitin-protein ligase HRD1 (SEL1L-HRD1) endoplasmic reticulum-associated degradation (ERAD) pathway (SEL1L-HRD1 ERAD pathway) modulation,
wherein the one or more nucleotide-based therapeutics comprise one or more antisense oligonucleotides (ASOs), one or more gapmer ASOs, one or more CRISPR interference constructs, or one or more nucleotide-based therapeutics that increase the expression of SEL1L, individually or in any combination of any thereof.

2. The composition of claim 1, wherein the one or more nucleotide-based therapeutics target a SEL1L pre-mRNA or mRNA.

3. The composition of claim 1, wherein the one or more ASOs comprise one or more nucleotides having at least 85% sequence identify to any one or more of SEQ ID NOs: 1-7.

4. The composition of claim 1, wherein the one or more gapmer ASOs comprise one or more nucleotides having at least 85% sequence identify to any one or more of SEQ ID NOs: 8-19.

5. The composition of claim 1, wherein the one or more CRISPR interference constructs comprise one or more nucleotides having at least 85% sequence identify to any one or more of SEQ ID NOs: 22-23.

6. The composition of claim 1, wherein the one or more nucleotide-based therapeutics that increase the expression of SEL1L comprise an SEL1L sequence or active fragment thereof having at least 85% sequence identify to SEQ ID NOs: 20 or 21

7. A vector, comprising a polynucleotide that encodes one or more nucleotide-based therapeutics of claim 1.

8. The vector of claim 7, wherein the vector is an adeno-associated viral (AAV) vector.

9. A pharmaceutical composition, comprising:
a composition of claim 1, and
a pharmaceutically-acceptable carrier.

10. A method of modulating SEL1L splicing or SEL1L expression, comprising:
administering one or more nucleotide-based therapeutics of claim 1 to a subject in need thereof.

11. The method of claim 9, wherein the ASOs are administered in an amount effective to modulate splicing of SEL1L mRNA or increase SEL1L expression.

12. The method of claim 9, wherein the subject in need thereof is a mammal, cell, or tissue, having or suspected of having one or more SEL1L mutations.

13. The method of claim 12, wherein the one or more SEL1L mutations are in exon 4 of SEL1L of the subject.

14. The method of claim 13, wherein the one or more SEL1L mutations comprise one or more of C141Y or S658P.

15. The method of claim 9, wherein the one or more nucleotide-based therapeutics are administered in an amount effective to improve any one or more of body weight, neurological symptoms, any one or more symptoms of ERAD deficiency, perinatal lethality, or B-cell lymphopenia.

16. A method of reducing one or more symptoms of an ERAD dysfunction-related disease, comprising:
administering one or more nucleotide-based therapeutics of claim 1 to a subject in need thereof, wherein the subject in need thereof has or is suspected of having ENDI-agammaglobulinemia (ENDI-A).

17. The method of claim 19, wherein the one or more nucleotide-based therapeutics target a SEL1L pre-mRNA or mRNA.

18. The method of claim 19, wherein the subject in need thereof has one or more SEL1L mutations in exon 4 of SEL1L.

19. The method of claim 18, wherein the one or more SEL1L mutations comprise one or more of C141Y or S658P.

20. The method of claim 19, wherein the one or more nucleotide-based therapeutics are administered in an amount affective to improve any one or more of: body weight, neurological symptoms, symptoms of developmental delay, intellectual disability, B cell lymphopenia and agammaglobuinemia, or axial hypotonia, individually or in any combination of any thereof.
